(19) United States
(12) Patent Application Publication
(10) Pub. No.: US 2002/0169394 A1
(43) Pub. Date: Nov. 14, 2002

(54) Integrated tissue poration, fluid harvesting and analysis device, and method therefor

(76) Inventors: Jonathan A. Eppstein, Atlanta, GA (US); Mark A. Samuels, Norcross, GA (US); Michael R. Hatch, Sugar Hill, GA (US); Difei Yang, Alpharetta, GA (US)

Correspondence Address: NEEDLE & ROSENBERG, P.C., The Candler Building, Suite 1200, 127 Peachtree Street, N.E., Atlanta, GA, 30303-1811, US

(21) Appl. No.: 10/084,763

(22) Filed: Feb. 21, 2002

Related U.S. Application Data
(63) Continuation-in-part of application No. 09/570,334, filed on May 15, 2000.
Application No. 09/570,334 is a continuation of application No. 09/208,166, filed on Dec. 9, 1998, now Pat. No. 6,142,939.
Application No. 09/208,166 is a continuation of application No. 08/776,863, filed on Sep. 5, 1997, now Pat. No. 5,885,211.
Application No. 08/776,863 is a continuation-in-part of application No. 08/520,547, filed on Aug. 29, 1995, now abandoned.
Application No. 08/520,547 is a continuation-in-part of application No. 08/152,442, filed on Nov. 15, 1993, now Pat. No. 5,458,140.
Application No. 08/520,547 is a continuation-in-part of application No. 08/152,174, filed on Dec. 8, 1993, now Pat. No. 5,445,611.
(63) Continuation-in-part of application No. 09/263,464, filed on Mar. 5, 1999.
(60) Provisional application No. 60/008,043, filed on Oct. 30, 1995.
(60) Provisional application No. 60/077,135, filed on Mar. 6, 1998.

(51) Int. Cl. A61B 5/00

(52) U.S. Cl. 600/573

(57) ABSTRACT

An integrated device (100, 200, 300, 400, 600, 1000) for harvesting a biological fluid from the tissue and analysis of the biological fluid. The device comprises a layer having a porating element disposed thereon to form at least one opening in the tissue. Biological fluid is harvested from the opening of the tissue and placed in contact with a sensor incorporated in the integrated device. The sensor is responsive to the biological fluid to provide an indication of a characteristic of the biological fluid, such as the concentration of an analyte in interstitial fluid. The porating element may comprise one or more heat conducting elements that are either optically or electrically heated, or one or more mechanical porating elements.

### CROSS REFERENCE TO RELATED APPLICATIONS

This application is a continuation-in-part of and claims benefit of priority from U.S. application Ser. No. 09/570,334, filed May 15, 2000, which is a continuation of and claims benefit of priority from U.S. application Ser. No. 09/208,166, filed Dec. 9, 1998, which is a continuation of and claims benefit of priority from U.S. application Ser. No. 08/776,863, filed Sep. 5, 1997 (now U.S. Pat. No. 5,885,211), which is a continuation-in-part of and claims benfit of priority from U.S. application Ser. No. 08/520,547, filed Aug. 29, 1995 (now abandoned), which is a continuation-in-part of and claims priority from U.S. application Ser. No. 08/152,442, filed Nov. 15, 1993 (now U.S. Pat. No. 5,458,140). This application is also a continuation-in-part of and claims priority from U.S. application Ser. No. 08/152,174, filed Dec. 8, 1993 (now U.S. Pat. No. 5,445,611); this application also claims the benefit of priority from U.S. Provisional Application No. 60/008,043, filed Oct. 30, 1995.

This application is also a continuation-in-part of and claims benefit of priority from U.S. application Ser. No. 09/263,464, filed Mar. 5, 1999, which claims benfit of priority from U.S. Provisional Application No. 60/077,135, filed Mar. 6, 1998.

All of the foregoing related applications are herein incorporated by reference in their entireties for all purposes.

### BACKGROUND OF THE INVENTION

The prevalence of diabetes has been increasing markedly in the world. At this time, diagnosed diabetics represent approximately 3% of the population of the United States. It is believed that the total actual number of diabetics in the United States is much higher. Diabetes can lead to numerous complications, such as, for example, retinopathy, nephropathy, and neuropathy.

The most important factor for reducing diabetes-associated complications is the maintenance of an appropriate level of glucose in the bloodstream. Proper maintenance of the level of glucose in the bloodstream may prevent and even reverse many of the effects of diabetes.

Traditional glucose monitoring devices operate on the principle of taking blood from an individual by a variety of methods, such as by needle or lancet. This is a multiple step process. First, a needle or lancet is used to make a hole in the individual's skin deep enough to obtain blood. Next, the individual applies a drop a blood to a strip that contains chemistry that interacts with the blood. Finally, the strip is inserted into a blood-glucose meter for measurement of glucose concentration based on a change in reflectance of the strip.

These traditional glucose monitoring systems require that an individual have separately available a needle or lancet for extracting blood, separately available strips carrying blood chemistry for creating a chemical reaction with respect to the glucose in the blood stream and changing color, and a blood-glucose meter for reading the change in color indicating the level of glucose in the bloodstream. The level of blood glucose, when measured by a glucose meter, is read from a strip carrying the blood chemistry through a well-known process.

There are other technologies being developed to provide an alternative to the conventional blood glucose monitoring procedures. One such technology involves measuring the level of glucose in interstitial fluid. In order to obtain samples of interstitial fluid, the barrier function of the stratum corneum must be overcome.

Published PCT application WO 9707734 entitled “Microporation Of Human Skin For Drug Delivery and Monitoring Applications,” to Eppstein et al., discloses a method of ablating the stratum corneum to form at least one micropore by treating a selected area of the stratum corneum with an effective amount of an optically absorbing compound, such as a dye, that exhibits strong absorption over the emission range of a pulsed light source and thermally ablating the stratum corneum by optically heating the dye. Heat is conductively transferred by the dye to the stratum corneum to elevate the temperature of tissue-bound water and other vaporizable substances in the selected area above the vaporization point of water and other vaporizable substances. Another microporation technique disclosed in that application involves the use of a solid thermal probe that is applied directly to the tissue. To the individual, these techniques are much less painful than using a lancet, if not completely painless.

Another technique for removing the stratum corneum is by direct absorption of optical energy. See, for example, U.S. Pat. No. 4,775,361 to Jacques et al.

In sum, there are several ways of making small holes in the tissue, including breaching the tissue mechanically with a needle or lancet, removing layers of tissue by thermal ablation techniques described above, or by the direct absorption of optical energy.

There is room for improving glucose monitoring technologies. In particular, it is desirable to integrate several functions of the glucose monitoring procedure into a single device. Preferably, this device would facilitate the harvesting of a biological fluid, such as interstitial fluid or blood, by making one or more small holes in the tissue, and the analysis of the biological fluid to determine a measure of a characteristic of the biological fluid, such as glucose level.

### SUMMARY OF THE INVENTION

Among other aspects, the present invention relates to methods for obtaining samples of biological fluids, including blood and interstitial fluid, for diagostic analysis/testing, and integrated devices for both (i) obtaining samples of biological fluids, such as interstitial fluid or blood, from tissue and (ii) analyzing/testing of the biological fluid.

As discussed above, one aspect of the invention relates to methods for obtaining biological fluids for analysis/testing.

For example, a suitable method for obtaining biological fluid for diagnostic testing comprises (a) forming an opening in an area of skin suitable for extracting a sample of biological fluid suitable for measuring a characteristic of the fluid; and (b) extracting the sample from the opening, wherein at least one of positive and negative pressure is employed in order to enhance the extraction of the sample.

In particular, one suitable method for obtaining interstitial fluid for diagnostic testing comprises (a) porating a selected area of skin to form an opening for extracting a sample comprising interstitial fluid, which sample is suitable for quantitating an analyte; and (b) collecting the sample from the opening, wherein step (b) is enhanced by applying a vacuum to the selected area of the skin.

In addition, the present invention includes methods for harvesting biological fluid from tissue and analyzing the biological fluid, comprising steps of: placing a layer in contact with a surface of tissue; forming at least one hole in the tissue; collecting biological fluid from the tissue through at least one opening in the layer; and wetting a sensor that is positioned in fluid communication with the at least one opening in the layer with biological fluid to measure a characteristic of the biological fluid. The at least one opening in the tissue is created by any of a variety of poration techniques, including thermal ablation, laser ablation, direct absorption ablation or mechanically creating a hole in the tissue with a mechanical porating element. A variety of techniques can be employed for enhancing the fluid collection in the integrated device, including the application of negative pressure, positive pressure, sonic energy, etc.

Another aspect of the inventive method relates to monitoring the concentration of an analyte in an individual's body comprising enhancing the permeability of the stratum corneum of a selected area of the individual's body surface to the analyte by (a) porating the stratum corneum of the selected area by means that form a micropore in the stratum corneum without causing serious damage to the underlying tissues, thereby reducing the barrier properties of the stratum corneum to the withdrawal of the analyte; (b) collecting a selected amount of the analyte; and (c) quantitating the analyte collected.

In one exemplary embodiment of this example of the invention, the method further comprises applying sonic energy to the porated selected area at a frequency in the range of about 5 kHz to 100 MHz, wherein the sonic energy is modulated by means of a member selected from the group consisting of frequency modulation, amplitude modulation, phase modulation, and combinations thereof. In another preferred embodiment, the method further comprises contacting the selected area of the individual's body with a chemical enhancer with the application of the sonic energy to further enhance analyte withdrawal.

Porating of the stratum corneum in this embodiment can be accomplished by any of a variety of means including (a) ablating the stratum corneum by contacting a selected area, up to about 1000 microns across of the stratum corneum with a heat source such that the temperature of tissue-bound water and other vaporizable substances in the selected area is elevated above the vaporization point of the water and other vaporizable substances thereby removing the stratum corneum in the selected area; (b) puncturing the stratum corneum with a micro-lancet calibrated to form a micropore of up to about 1000 microns in diameter; (d) ablating the stratum corneum by focusing a tightly focused beam of sonic energy onto the stratum corneum; (d) hydraulically puncturing the stratum corneum with a high pressure jet of fluid to form a micropore of up to about 1000 microns in diameter and (e) puncturing the stratum corneum with short pulses of electricity to form a micropore of up to about 1000 microns in diameter.

One embodiment relating to thermally ablating the stratum corneum comprises treating at least the selected area with an effective amount of a dye that exhibits strong absorption over the emission range of a pulsed light source and focusing the output of a series of pulses from the pulsed light source onto the dye such that the dye is heated sufficiently to conductively transfer heat to the stratum corneum to elevate the temperature of tissue-bound water and other vaporizable substances in the selected area above the vaporization point of the water and other vaporizable substances. Preferably, the pulsed light source emits at a wavelength that is not significantly absorbed by skin. For example, the pulsed light source can be a laser diode emitting in the range of about 630 to 1550 nm, a laser diode pumped optical parametric oscillator emitting in the range of about 700 and 3000 nm, or a member selected from the group consisting of arc lamps, incandescent lamps, and light emitting diodes.

A sensing system for determining when the barrier properties of the stratum corneum have been surmounted can also be provided. One example of a sensing system comprises light collection means for receiving light reflected from the selected area and focusing the reflected light on a photodiode, a photodiode for receiving the focused light and sending a signal to a controller wherein the signal indicates a quality of the reflected light, and a controller coupled to the photodiode and to the pulsed light source for receiving the signal and for shutting off the pulsed light source when a preselected signal is received.

In another embodiment, the method further comprises cooling the selected area of stratum corneum and adjacent skin tissues with cooling means such that said selected area and adjacent skin tissues are in a selected precooled, steady state, condition prior to poration.

In still another embodiment, the method comprises ablating the stratum corneum such that interstitial fluid exudes from the micropores, collecting the interstitial fluid, and analyzing the analyte in the collected interstitial fluid. After the interstitial fluid is collected, the micropore can be sealed by applying an effective amount of energy from the laser diode or other light source such that interstitial fluid remaining in the micropore is caused to coagulate. Preferably, vacuum can be applied to the porated selected area to enhance collection of interstitial fluid.

In yet another embodiment, the method comprises, prior to porating the stratum corneum, illuminating at least the selected area with unfocused light from the pulsed light source such that the selected area illuminated with the light is sterilized.

Another suitable method of porating the stratum corneum comprises contacting the selected area with a metallic wire such that the temperature of the selected area is raised from ambient skin temperature to greater than 100° C. within about 10 to 50 msec and then returning the temperature of the selected area to approximately ambient skin temperature within about 30 to 50 msec, wherein this cycle of raising the temperature and returning to approximately ambient skin temperature is repeated a number of times effective for reducing the barrier properties of the stratum corneum.

Preferably, the step of returning to approximately ambient skin temperature is carried out by withdrawing the wire from contact with the stratum corneum. It is also preferred to provide means for monitoring electrical impedance between the wire and the individual's body through the selected area of stratum corneum and adjacent skin tissues and means for advancing the position of the wire such that as the ablation occurs with a concomitant reduction in resistance, the advancing means advances the wire such that the wire is in contact with the stratum corneum during heating of the wire. Further, it is also preferred to provide means for withdrawing the wire from contact with the stratum corneum, wherein the monitoring means is capable of detecting a change in impedance associated with contacting an epidermal layer underlying the stratum corneum and sending a signal to the withdrawing means to withdrawn the wire from contact with the stratum corneum. The wire can be heated by an ohmic heating element, can have a current loop having a high resistance point wherein the temperature of the high resistance point is modulated by passing a modulated electrical current through said current loop to effect the heating, or can be positioned in a modulatable alternating magnetic field of an excitation coil such that energizing the excitation coil with alternating current produces eddy currents suffient to heat the wire by internal ohmic losses.

The present invention also relates to methods for enhancing the transdermal flux rate of an active permeant into a selected area of an individual's body comprising the steps of enhancing the permeability of the stratum corneum layer of the selected area of the individual's body surface to the active permeant by means of

(a) porating the stratum corneum of the selected area by means that form a micro-pore in the stratum corneum without causing serious damage to the underlying tissues and thereby reduce the barrier properties of the stratum corneum to the flux of the active permeant; and

(b) contacting the porated selected area with a composition comprising an effective amount of the permeant such that the flux of the permeant into the body is enhanced.

In one embodiment, the method further comprises applying sonic energy to the porated selected area for a time and at an intensity and a frequency effective to create a fluid streaming effect and thereby enhance the transdermal flux rate of the permeant into the body.

A method is also provided for appyling a tatoo to a selected area of skin on an individual's body surface comprising the steps of:

(a) porating the stratum corneum of the selected area by means that form a micro-pore in the stratum corneum without causing serious damage to the underlying tissues and thereby reduce the barrier properties of the stratum corneum to the flux of a permeant; and

(b) contacting the porated selected area with a composition comprising an effective amount of a tattoing ink as a permeant such that the flux of said ink into the body is enhanced.

A method is still further provided for reducing a temporal delay in diffusion of an analyte from blood of an individual to said individual's interstitial fluid in a selected area of skin comprising applying means for cooling to said selected area of skin.

A method is yet further provided for reducing evaporation of interstitial fluid and the vapor pressure thereof, wherein said interstitial fluid is being collected from a micropore in a selected area of stratum corneum of an individual's skin, comprising applying means for cooling to said selected area of skin.

As discussed above, the present invention also relates to devices, including integrated devices for harvesting biological fluid, such as interstitial fluid or blood, from tissue and analyzing at least a portion of the harvested biological fluid.

One embodiment of a multi-layer integrated device comprises (a) a receiving layer capable of receiving a biological fluid including an analyte and facilitating the movement of the fluid; (b) an analyte sensor capable of detecting the presence of analyte or measuring the concentration of analyte in the fluid; and (c) a substrate layer that is in contact with a processing circuit, wherein the receiving layer (a) is located underneath at least a portion of the substrate layer (c) and facilitates the movement of the biological fluid to the sensor (b); and further wherein said substrate layer (c) has at least one opening therein.

Another embodiment of the multi-layer integrated device comprises (a) a receiving layer capable of receiving a biological fluid including an analyte and facilitating the movement of the fluid; (b) an analyte sensor capable of detecting the presence of analyte or measuring the concentration of analyte in the fluid; (c) a substrate layer that is in contact with a processing circuit, and (d) a bottom layer; wherein the receiving layer (a) is located underneath at least a portion of the substrate layer (c) and facilitates the movement of the biological fluid to the sensor (b); and further wherein said substrate layer (c) has at least one opening therein.

The inventive integrated devices can also include features by which the analyte is detected and/or measured and features by which the results of the detection and/or measurement of the analyte are displayed by the device.

To this end, yet another inventive integrated device comprises a pad capable of receiving and transporting a sample of biological fluid (such as blood or interstitial fluid) containing an analyte; a detector for detecting the presence and/or quantitating the concentration of analyte in the sample, said detector being capable of being in contact with a display for illustrating results of the detector; and a strap or adhesive tape for holding the pad to an area of skin surface, wherein the integrated device contains at least one opening suitable to allow the sample of biological fluid to contact the pad.

Still another inventive integrated device for removing and testing a sample of biological fluid (such as blood or interstitial fluid) from the skin comprises a lower section in contact with the skin, said section having at least one opening therein; a pad capable of collecting and transporting a sample of biological fluid containing an analyte; and a detector for determining the presence and/or quantity of the analyte, said detector capable of being in contact with a display for the results of the detector.

As can be seen, the present invention includes a number of integrated devices.

For example, the integrated device can comprise at least a first layer that supports a porating element, and which is to be placed in physical contact with the tissue surface. An optional second layer overlies the first layer with a space therebetween. A sensor can be disposed between the first and second layers, or otherwise at a location on or about the first layer so as to be wetted for harvesting biological fluid. The porating element takes on one of several forms, such as a mechanical porating element or a heat conducting element that is heated either by the application of electrical energy or by the application of optical energy. In the case where the porating element is a heat conducting element, the heat conducting element (or probe) heats up and transfers thermal energy by conduction to the tissue to which the device is applied, such as skin. The tissue is ablated so as to form at least one opening or micropore therein. Interstitial fluid, or if the opening is deep enough, blood, is collected from the opening formed in the tissue. In addition, the surface tension of the collected fluid in the integrated device is manipulated to enhance and expedite the overall analysis process. For example, an optional wicking mesh or a capillary channel is provided to facilitate the flow of biological fluid to the sensor.

The above and other aspects and advantages of the present invention will become more readily apparent when reference is made to the following description, taken in conjunction with the accompanying drawings.

### BRIEF DESCRIPTION OF THE DRAWINGS

FIG. 1 is a diagram of an analyte assay system including an integrated device according to one embodiment of the present invention.

FIG. 2 is a diagram of an analyte assay system including an integrated device according to another embodiment of the present invention.

FIG. 3 is a partial cross-sectional view of the integrated device according to the first embodiment showing how biological fluid is collected and delivered to an assay pad.

FIG. 4 is a top view showing the arrangement of the sense electrodes and sensor of the integrated device according to the first embodiment.

FIG. 5 is a side view of the integrated device showing the application of a deformation force to the integrated device of the first embodiment.

FIG. 6 is a comprehensive diagram of an analyte assay system and showing a cross-sectional view of an integrated device according to still another embodiment of the present invention.

FIG. 7 is a side cross-sectional view of the integrated device shown in FIG. 6.

FIG. 8 is a perspective view of a hand-held assay unit and integrated device associated therewith according to still another embodiment of the present invention.

FIG. 9 is a perspective view of the integrated device according shown in FIG. 8.

FIG. 10 is a cross-sectional view taken through line 10-10 of FIG. 9.

FIG. 11 is a cross-sectional view of an integrated device according to another embodiment of the present invention.

FIG. 12 is a bottom view of the integrated device of FIG. 11.

FIG. 13 is a side cross-sectional view of an integrated device having a sensor which is optically read.

FIG. 14 is a top view of an integrated device that is optically read.

FIG. 15 is a top view of a pneumatic sealing system used in conjunction with an integrated device.

FIG. 16 is a side view of a pneumatic sealing system shown in FIG. 15.

FIG. 17 is an enlarged side view of the use of a mechanical pressure device with an integrated device.

FIG. 18 is an enlarged side view showing the effects of the mechanical pressure device shown in FIG. 17.

FIGS. 19 and 20 are schematic diagrams showing the application of sonic energy in conjunction with the integrated device.

FIG. 21 is a side view of an integrated device according to another embodiment of the invention in which the poration elements are mechanical porating elements.

FIG. 22 is a partial perspective view showing the integrated device of FIG. 21 in greater detail.

FIG. 23 shows a schematic representation of a system for delivering laser diode light and monitoring the progress of poration according to an embodiment of the invention.

FIG. 24 shows a schematic representation of a closed-loop feedback system for monitoring poration according to an embodiment of the invention.

FIG. 25A shows a schematic representation of an optical poration system comprising a cooling device according to an embodiment of the invention.

FIG. 25B shows a top view of a schematic representation of an illustrative cooling device according to FIG. 25A according to an embodiment of the invention.

FIG. 26 shows a schematic representation of an ohmic heating device with a mechanical actuator.

FIG. 27 shows a schematic representation of a high resistance current loop heating loop device.

FIG. 28 shows a schematic representation of a device for modulating heating using inductive heating.

FIG. 29 shows a schematic representation of a closed loop impedance monitor using changes in inpedance to determine the extent of poration.

FIGS. 30A-D show cross sections of human skin treated with copper phthalocyanine and then subjected, respectively, to 0, 1, 5, and 50 pulses of 810 nm light with an energy density of 4000 Am for a pulse period of 20 ms.

FIGS. 31-33 show graphic representations of temperature distribution during simulated thermal poration events using optical poration.

FIGS. 34 and 35 show graphic representations of temperature as a function of time in the stratum corneum and viable epidermis, respectively, during simulated thermal poration events using optical poration.

FIGS. 36-38 show graphic representations of temperature distribution, temperature as a function of time in the stratum corneum, and temperature as a function of time in the viable epidermis, respectively, during simulated thermal poration events using optical poration wherein the tissue was cooled prior to poration.

FIGS. 39-41 show graphic representations of temperature distribution, temperature as a function of time in the stratum corneum, and temperature as a function of time in the viable epidermis, respectively, during simulated thermal poration events wherein the tissue was heated with a hot wire.

FIGS. 42-44 show graphic representations of temperature distribution, temperature as a function of time in the stratum corneum, and temperature as a function of time in the viable epidermis, respectively, during simulated thermal poration events wherein the tissue was heated with a hot wire and the tissue was cooled prior to poration.

FIGS. 45 and 46 show graphic representations of temperature distribution and temperature as a function of time in the stratum corneum, respectively, during simulated thermal poration events wherein the tissue is heated optically according to the operating parameters of Tankovich '803.

FIG. 47 shows a graphic representation of interstitial fluid (ISF) and blood glucose levels as a function of time.

FIG. 48 shows a scatter plot representation of the difference term between the ISF glucose and the blood glucose data of FIG. 47.

FIG. 49 shows a histogram of the relative deviation of the ISF to the blood glucose levels from FIG. 47.

FIG. 50 shows a cross section of an illustrative delivery apparatus for delivering drug to a selected area on an individual's skin.

FIGS. 51A-C show graphic representations of areas of skin affected by delivery of lidocaine to selected areas where the stratum corneum is porated (FIGS. 29A-B) or not porated (FIG. 29C).

FIG. 52 shows a plot comparing the amount of interstitial fluid harvested from micropores with suction alone and with a combination of suction and ultrasound.

FIGS. 53, 54, and 55 show a perspective view of an ultrasonic transducer/vacuum apparatus for harvesting interstitial fluid, a cross section view of the same apparatus, and cross sectional schematic view of the same apparatus, respectively.

FIGS. 56A-B show a top view of a handheld ultrasonic transducer and a side view of the spatulate end thereof, respectively.

FIGS. 57A and 57B illustrate integrated device with the added feature that the analysis is performed at the time of collection of the analyte.

FIG. 58 shows an embodiment of a portable monitoring device for using this method of collecting and monitoring analytes with the aid of ultrasound and chemical enhancer.

FIG. 59A and 59B show an illustrative embodiment of a portable device for collection of an analyte with the aid of ultrasound and chemical enhancer.

### DETAILED DESCRIPTION

As discussed above, the present invention relates to methods for obtaining biological fluids, including blood and interstitial fluid, for diagostic analysis/testing, and integrated devices for both (i) obtaining biological fluids, such as interstitial fluid or blood, from tissue and (ii) analyzing/testing of the fluid.

In this regard, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise. Thus, for example, reference to a method for delivery of “a drug” includes reference to delivery of a mixture of two or more drugs, reference to “an analyte” includes reference to one or more of such analytes, and reference to “a permeation enhancer” includes reference to a mixture of two or more permeation enhancers.

As used herein, the expression “biological fluid” is intended to include blood, e.g., blood serum or whole blood, as well as interstitial fluid. “Interstitial fluid” is the clear fluid that occupies the space between the cells in the body.

As used herein, “analyte” means any chemical or biological material or compound suitable for passage through a biological membrane by the technology taught in this present invention, or by technology previously known in the art, of which an individual might want to know the concentration or activity inside the body. Glucose is a specific example of an analyte because it is a sugar suitable for passage through the skin, and individuals, for example those having diabetes, might want to know their blood glucose levels. Other examples of analytes include, but are not limited to, such compounds as sodium, potassium, bilirubin, urea, ammonia, calcium, lead, iron, lithium, salicylates, and the like.

As used herein, the term “tissue” means an aggregate of cells of a particular kind, together with their intercellular substance, that forms a structural material. At least one surface of the tissue must be accessible to electromagnetic radiation so that one embodiment of the invention can be carried out. The preferred tissue is the skin. Other tissues suitable for use with this invention include mucosal tissue and soft organs.

As used herein, a “biological membrane” is intended to mean a membrane material present within a living organism that separates one area of the organism from another and, in many instances, that separates the organism from its outer environment. Skin and mucous membranes are thus included.

The term “stratum corneum” means the outermost layer of the skin, consisting of from about 15 to about 20 layers of cells in various stages of drying out. The stratum corneum provides a barrier to the loss of water from inside the body to the external environment and from attack from the external environment to the interior of the body. The term “epidermis” means the metabolically active region of the skin. It is found just below the stratum corneum and is approximately 10 times as thick as the stratum corneum. The epidermis does not contain blood transport structures, i.e., capillaries. The term “dermis” means the region of skin approximately 10 times as thick as the epidermis and found just below the epidermis. The dermis contains large amounts of collagen, which provides structural integrity to the skin. The dermis contains a layer of small blood capillaries that provide oxygen and nutrients to the rest of the layers of skin.

As used herein, “individual” and “organism” refer to both humans and animals, to which the present invention may be applied.

As used herein, “ablation” refers to the process of controlled removal of a selected area of tissue from the surrounding tissue by kinetic energy released when the temperature of vaporizable substances in the selected area is rapidly elevated above the vaporization point thereby flash vaporizing some of the tissue in the selected area.

As used herein “puncture” or “micro-puncture” means the use of mechanical, hydraulic, or electrical means to perforate the stratum corneum.

To the extent that “ablation” and “puncture” accomplish the same purpose of poration, i.e. the creating a hole or pore in the stratum corneum without significant damage to the underlying tissues, these terms may be used interchangeably.

As used herein, “poration,” “microporation,” or any such similar term means the formation of a small hole, opening or pore to a desired depth in or through a biological membrane, such as skin or mucous membrane, or the outer layer of an organism to lessen the barrier properties of this biological membrane to the passage of biological fluids, such as analytes from within the biological membrane or the passage of permeants or drugs from without the biological membrane into the body for selected purposes, or for certain medical or surgical procedures. The size of the hole or “micropore” so formed is approximately 1-1000 μm in diameter. It is to be understood that the term “micropore” is used in the singular form for simplicity, but that it multiple openings or pores may be formed by the integrated device and/or method according to the present invention.

As used herein, “non-invasive” means not requiring the entry of a needle, catheter, or other invasive medical instrument into a part of the body.

As user herein, “minimally invasive” refers to the use of mechanical, hydraulic, or electrical means that invade the stratum corneum to create a small hole or micropore without causing substantial damage to the underlying tissues.

As used herein, the term “integrated device” means a device suitable for forming small holes or micropores in tissue, collecting a biological fluid from the tissue (preferably through the micropores so created) and analyzing the biological fluid to determine a characteristic thereof.

As used herein, “sonic energy” refers to mechanical pressure waves with frequencies from 10 Hz to 1000 MHz.

The term “porating element” is meant to include any means of forming a micropore, hole or opening described above, including by thermal ablation, mechanically breaching the tissue by lancet or needle, and other known techniques. An example of a mechanical porating element is disclosed in commonly assigned published PCT application WO 9800193, entitled, “Multiple Mechanical Microporation Of Skin Or Mucosa.”

The term “heated probe” or “heat conducting element” refers to a probe, preferably solid phase, which is capable of being heated in response to the application of electrical or electromagnetic (optical) energy thereto for achieving thermal ablation of the tissue. For simplicity, the probe is referred to as a “heated probe” or “heatable probe” which includes a probe in a heated or unheated state, but which is heatable.

As used herein, “penetration enhancement” or “permeation enhancement” means an increase in the permeability of skin to a drug, analyte, dye, stain, or other chemical molecule (also called “permeant”), i.e., so as to increase the rate at which a drug, analyte, or chemical molecule permeates the stratum corneum and facilitates the poration of the stratum corneum, the withdrawal of analytes out through the stratum corneum or the delivery of drugs through the stratum corneum and into the underlying tissues. The enhanced permeation effected through the use of such enhancers can be observed, for example, by observing diffusion of a dye, as a permeant, through animal or human skin using a diffusion apparatus.

As used herein, “chemical enhancer,” “penetration enhancer,” “permeation enhancer,” and the like includes all enhancers that increase the flux of a permeant, analyte, or other molecule across the skin, and is limited only by functionality. In other words, all cell envelope disordering compounds and solvents and any other chemical enhancement agents are intended to be included.

As used herein, “dye,” “stain,” and the like shall be used interchangeably and refer to a biologically suitable chromophore that exhibits strong absorption at the emission range of a pulsed light source used to ablate tissues of the stratum corneum to form micropores therein.

As used herein, “transdermal” or “percutaneous” means passage of a permeant into and through the skin to achieve effective therapeutic blood levels or deep tissue levels of a drug, or the passage of a molecule present in the body (“analyte”) out through the skin so that the analyte molecule may be collected on the outside of the body.

As used herein, the term “permeant,” “drug,” or “pharmacologically active agent” or any other similar term means any chemical or biological material or compound suitable for transdermal administration by the methods previously known in the art and/or by the methods taught in the present invention, that induces a desired biological or pharmacological effect, which may include but is not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating the disease from the organism. The effect may be local, such as providing for a local anaesthetic effect, or it may be systemic. This invention is not drawn to novel permeants or to new classes of active agents. Rather it is limited to the mode of delivery of agents or permeants that exist in the state of the art or that may later be established as active agents and that are suitable for delivery by the present invention.

Such substances include broad classes of compounds normally delivered into the body, including through body surfaces and membranes, including skin. In general, this includes but is not limited to: anti-infectives such as antibiotics and antiviral agents; analgesics and analgesic combinations; anorexics; anti-helminthics; anti-arthritics; anti-astlunatic agents; anti-convulsants; anti-depressants; anti-diabetic agents; anti-diarrheals; anti-histamines; anti-inflammatory agents; anti-migraine preparations; anti-nauseants; anti-neoplastics; anti-Parkinson's drugs; anti-pruritics; anti-psychotics; anti-pyretics; anti-spasmodics; anti-cholinergics; sympathomimetics; xanthine derivatives; cardiovascular preparations including potassium and calcium channel blockers, beta-blockers, alpha-blockers, and antiarrhythmics; antihypertensives; diuretics and antidiuretics; vasodilators including general coronary, peripheral and cerebral; central nervous system stimulants; vasoconstrictors; cough and cold preparations, including decongestants; hormones such as estradiol and other steroids, including corticosteroids; hypnotics; immunosuppressives; muscle relaxants; parasympatholytics; psychostimulants; sedatives; and tranquilizers. By the method of the present invention, both ionized and nonionized drugs may be delivered, as can drugs of either high or low molecular weight.

As used herein, an “effective” amount of a pharmacologically active agent means a sufficient amount of a compound to provide the desired local or systemic effect and performance at a reasonable benefit/risk ratio attending any medical treatment. An “effective” amount of a permeation or chemical enhancer as used herein means an amount selected so as to provide the desired increase in skin permeability and the desired depth of penetration, rate of administration, and amount of drug delivered.

As used herein, “carriers” or “vehicles” refer to carrier materials without significant pharmacological activity at the quantities used that are suitable for administration with other pharmaceutically active materials, and include any such materials known in the art, e.g., any liquid, gel, solvent, liquid diluent, solubilizer, or the like, that is nontoxic at the quantities employed and does not interact with the drug to be administered in a deleterious manner. Examples of suitable carriers for use herein include water, mineral oil, silicone, inorganic gels, aqueous emulsions, liquid sugars, waxes, petroleum jelly, and a variety of other oils and polymeric materials.

As used herein, “transdermal flux rate” is the rate of passage of any analyte out through the skin of an individual, human or animal, or the rate of passage of any drug, pharmacologically active agent, dye, or pigment in and through the skin of an individual, human or animal.

As used herein, the terms “intensity amplitude,” “intensity,” and “amplitude” are used synonymously and refer to the amount of energy being produced by the sonic energy system.

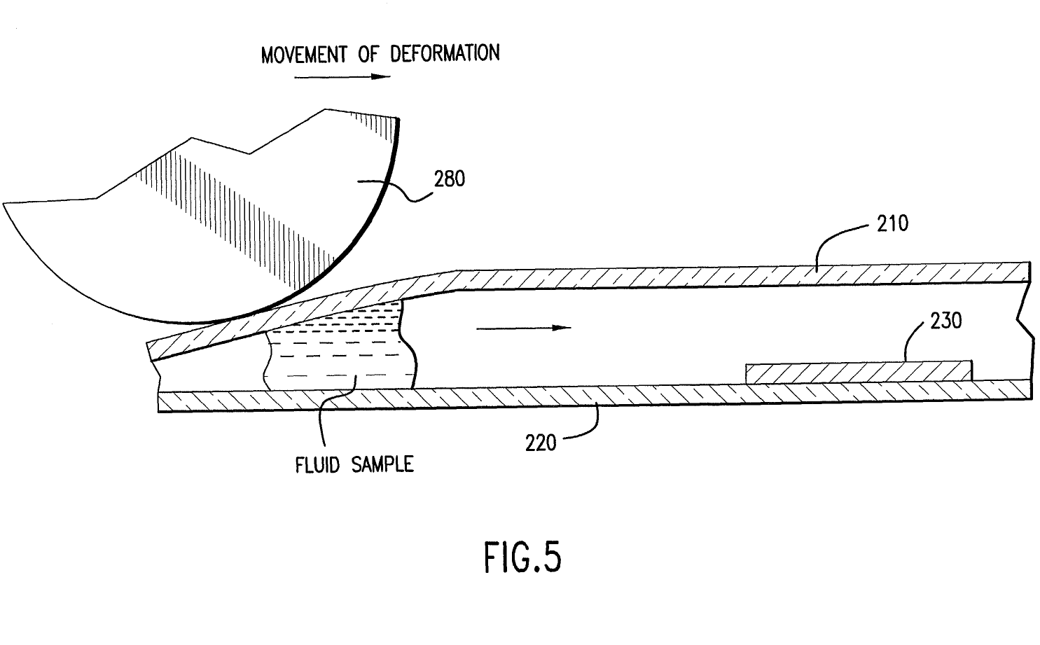

As used herein, “frequency modulation” or “sweep” means a continuous, graded or stepped variation in the amplitude or frequency of ultrasound in a given time period. A frequency modulation is a graded or stepped variation in frequency in a given time period, for example 5.4-5.76 MHz in Isec., or 5-10 MHz in 0.1 sec., or 10-5 MHz in 0.1 sec., or any other frequency range or time period that is appropriate to a specific application. A complex modulation can include varying both the frequency and intensity simultaneously. For example, FIGS. 4A and 4B of U.S. Pat. No. 5,458,140 could, respectively, represent amplitude and frequency modulations being applied simultaneously to a single sonic energy transducer.

As used herein “phase modulation” means the timing of the signal has been changed relative to its initial state shown in FIG. 4C of U.S. Pat. No. 5,458,140. The frequency and amplitude of the signal can remain the same. A phase modulation can be implemented with a variable delay such as to selectively retard or advance the signal temporarily in reference to its previous state, or to another signal. The sonic energy, in its various applications such as with frequency, intensity or phase modulation, or combinations thereof and the use of chemical enhancers combined with modulated sonic energy, as described herein, can vary over a frequency range of between about 5 kHz to 100 MHz, with a range of between about 20 kHz and 30 MHz being preferred.

As discussed above, the present invention includes methods for harvesting biological fluids from tissue and analyzing at least a portion of the harvested biological fluid.

These methods typically include the steps of placing a layer in contact with a surface of tissue; forming at least one hole in the tissue; collecting biological fluid from the tissue through at least one opening in the layer; and wetting a sensor that is positioned in fluid communication with the at least one opening in the layer with biological fluid to measure a characteristic of the biological fluid. The technique for forming the opening in the tissue may be a mechanical element (lancet, needle, etc.), an electrically heated poration element, an optically heated poration element, etc. In addition, the opening in the tissue may be formed adjacent to an edge of the layer, whereby biological fluid enters the layer through a capillary feed channel formed between the top and bottom layers of the device, as described above.

In addition, the present invention relates to integrated poration harvesting and analysis devices for biological fluids.

Exemplary devices can include a first layer having a porating element disposed thereon, the porating element forming at least one opening in the tissue; a sensor positioned in fluid communication with the at least one opening in the tissue, the sensor being responsive to a biological fluid collected from the tissue to provide an indication of a characteristic of the biological fluid. Similarly, the present invention is directed to an integrated fluid harvesting and analyis device comprising a first layer for positioning in contact with tissue and through which poration of tissue is achieved such that at least one opening is formed in the first layer and at least one opening is formed in the tissue; a sensor positioned in fluid communication with the at least one opening of the first layer, the sensor being responsive to a biological fluid collected from the tissue to provide an indication of a characteristic of the biological fluid. A quantity of photothermal material is disposed on a portion of the first layer, which is heated by optical energy to form the micropore (opening) in the tissue, and thereby create the opening in the first layer.

Several embodiments of suitable integrated devices are disclosed herein. These embodiments will now be discussed in greater detail.

In certain embodiments, a porating element is provided that is used for forming at least one opening in the tissue and in the layer that is in contact with the skin. In some of these embodiments, the porating element is a heated probe or heat conducting element which, when heated, forms at least one opening, i.e., a micropore, in the tissue. What is common among these embodiments is that the heated probe is heated such that the temperature of tissue-bound water and other vaporizable substances in a selected area of the surface of the tissue, such as the stratum corneum, is elevated above the vaporization point of water and other vaporizable substances thereby removing the surface of the tissue in the selected area. Consequently, the heated probe forms a micropore in the surface of the tissue approximately 1-1000 μm in diameter.

Some of the microporation techniques described herein are further described in published PCT application WO 9707734, the entirety of which is incorporated herein by reference.

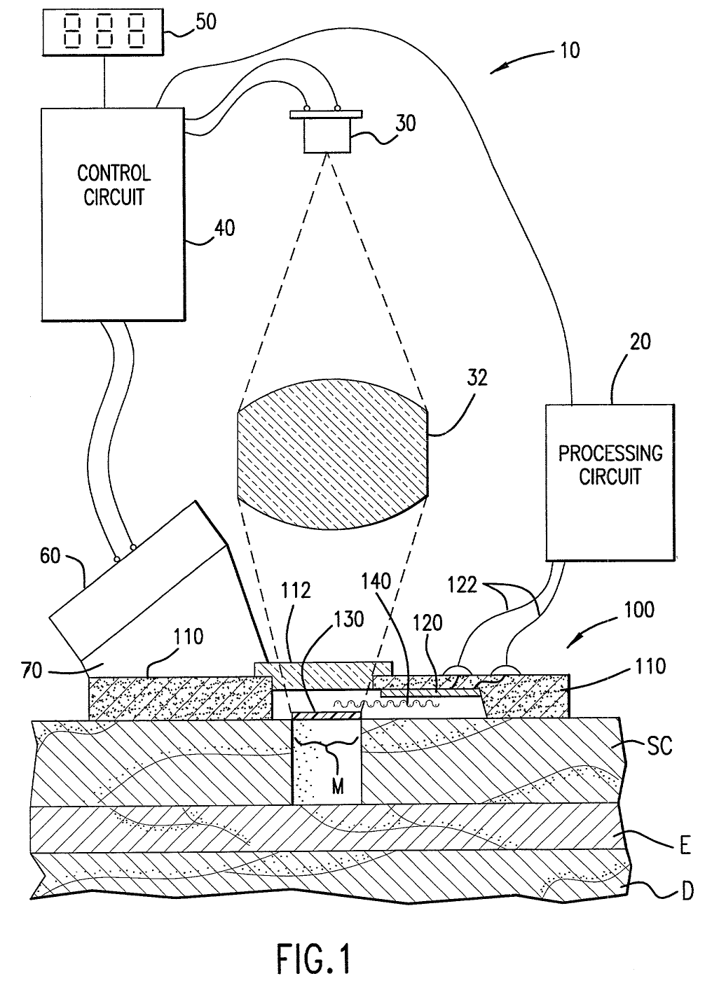

For example, referring first to FIG. 1, an analyte assay system is shown at reference numeral 10 comprising an integrated device 100. The configuration of the integrated device 100 is shown in a simplified manner so as to illustrate several basic elements of the inventive integrated device. The integrated device 100 comprises a substrate layer 110 that includes an optically transparent window 112 on at least a portion thereof. An analyte sensor 120 is disposed on an under-surface of the substrate layer 110. In the embodiment of the invention where the analyte sensor 120 is an electrochemical biosensor, the integrated device 100 has electrode leads 122 that connect to the analyte sensor 120 and to a processing circuit 20.

A layer of photothermal material 130 is provided on the bottom surface of the substrate 110 or directly applied to the tissue surface from which biological fluid is to be collected. In addition, a layer of adhesive may be applied to certain bottom surfaces of the substrate 110 to hold the integrated device onto the tissue surface and prevent biological fluid from being drawn between the tissue surface and the bottom layer of the integrated device 110. The integrated device 100 could be a one-use disposable element, or could be suitable for multiple uses. The photothermal material layer 130 also serves to seal the bottom surface of the integrated device 100 to protect the analyte sensor 120 from the external environment. Suitable compounds for the photothermal material are described in the aforementioned published PCT application, WO 9707734, and in U.S. Provisional Application No. 60/077,135, which is incorporated herein by reference. Alternatively, a hole or opening may be provided at the location of the optically transparent window 112, and a quantity of photothermal material is disposed directly on the tissue surface, or on a bottom surface of the integrated device 100.

The integrated device 100 and all other specific embodiments described hereinafter, are designed to form micropores in tissue, collect fluid from the tissue, and analyze the fluid in a single (integrated) step. As an example, FIG. 1 shows a stratum corneum layer SC and epidermis E of skin. The micropores may be of a depth that extends into the stratum corneum SC, or may extend through the stratum corneum to (and into) the epidermis layer E. The micropores may go still further through the epidermis layer E into the vascularized dermal layer to obtain whole blood as the fluid sample, rather than just interstitial fluid.

In operation, optical energy from a source 30, such as a laser diode, is projected through the optical window 112 of the integrated device 100. A focusing lens 32 may be provided to focus the beam onto the integrated device 100. The optical energy from the source 30 is focused onto the layer of photothermal material 130. The photothermal material 130 heats up in response to absorbing the optical energy and transfers heat to the surface of the tissue. Once sufficient heat is transferred to the surface of the tissue, a micropore is formed in the tissue, such as into the stratum corneum SC. In the process, the photothermal material 130 vaporizes together with the layers of stratum corneum that are affected by the heat conducted from the optically heated photothermal material 130. Consequently, the micropore M formed in the tissue creates a path into the integrated device 100. Similar size openings are formed in the photothermal material 130 at the bottom portion of the integrated device 100 so as to permit flow of fluid into the integrated device 100.

More specifically, the micropore M can permit interstitial fluid in the tissue to flow into the integrated device 100 and eventually to contact the sensor 120. The sensor 120 then reacts to the interstitial fluid to measure a concentration of an analyte, such as glucose. The processing circuit 20 is any well known glucose measuring circuit that is capable of measuring the output of an electrochemical analyte sensor and producing a reading correlated to the concentration of a target analyte in biological fluid, such as glucose.

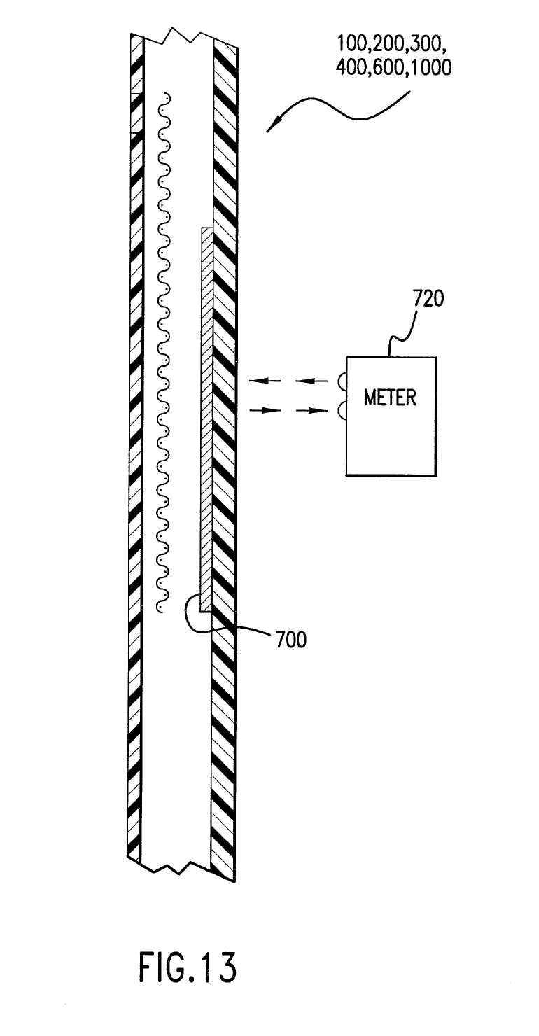

Alternatively, as shown in FIG. 13, a calorimetric assay system may be employed, instead of the electrochemical one shown in the version of the integrated device of FIG. 1. The calorimetric assay system used in conjunction with an integrated device is described hereinafter in conjunction with FIG. 13.

A control circuit 40 may be provided that is connected to the source 30 and to the processing circuit 20. The control circuit 40 may further process the assay measurement made by the processing circuit 20 to drive a display 50 in order to display the assay measurement. In addition, as an optional enhancement, sonic energy may be applied to the microporated tissue by a sonic transducer 60 that is coupled to the tissue by a portion of the substrate layer 110 that is made of suitable acoustically coupling material. The sonic transducer 60 may be a piezoelectric device, a magneto-restrictive device or a small electromagnetic transducer, such as a miniature audio speaker element of a moving coil, moving magnet or electrostatic design. The sonic energy coupled to the tissue acts as a driving force to direct interstitial fluid into the integrated device 100. Moreover, the sonic transducer 60 may be comprised of one or more separate elements each of which may be individually controlled to achieve different effects on the focus and energy density of the sonic energy within the system.

The sonic energy may be focused or formed by a beam former 70. The beam forming may be achieved via a combination of one or more elements 70 which are placed in the propagation path of the sonic waves emitted by the transducer 60. By selectively altering the propagation velocity of the sonic waves, these elements can be shown to produce a redirection of this energy and if desired a focusing can be achieved, similar to the way diffractive elements in an optical lens system effect the light waves passing through them. A plano-concave aluminum element may be placed on the surface of the transducer. The radius of the concave cutout nominally defines the focal point of the system. Alternatively, a focus of sonic energy may be achieved by using multiple sonic sources operated in a coordinated fashion to form a phased array wherein energy peaks and nulls are defined by the additive superpositioning of the sonic energy waves.

With a sonic energy system, one can also easily create a standing wave pattern wherein the natural resonance of the system creates localized stationary energy peaks. Once a standing wave has been established, only a small amount of additional energy is needed to maintain it at this same amplitude. Also, subtle perturbations in the system such as a small shift in frequency or a separate action that affects the systems natural resonance can cause the standing wave to move in a controllable and predictable fashion allowing manipulation of the fluid sample as desired. Using multiple transducers, one can also establish a standing wave and easily control the position, amplitude and period between wave peaks.

Further, vacuum (negative pressure) may be applied to the microporated site to assist in the harvesting of the biological fluid into the integrated device so as to make contact with the sensor. Likewise, positive pressure may be applied to the integrated device 100 with a downward force on the integrated device 100 to force fluid to move towards the sensor 120. The application of positive pressure is described in more detail hereinafter.

To enhance the flow of the fluid to the sensor 120, the surface tension effects may be employed. For example, surfactant compounds are optionally applied to surfaces of the integrated device 100 to direct fluid flow to the sensor. Furthermore, a mesh 140 may be provided in the integrated device 100 to wick interstitial fluid towards the sensor 120. The mesh 140 is positioned and clamped between top and bottom layers of the integrated device, or may be held in place by small thermal welds, glue, or mechanical spacers. The mesh 140 acts by a surface tension mechanism to move the biological fluid to the sensor. Still further, a capillary channel may be formed between the top and bottom layers of the integrated device 100, thereby creating surface tension effects to move the fluid to the sensor 120.

The mesh 140 may be treated with a surfactant compound as well. Further still, surfaces of the integrated device 100 where it is desired that interstitial not flow may be treated with hydrophobic compounds. The mesh 140 will also displace volume in the integrated device to thereby reduce the volume of interstitial fluid needed for an adequate assay measurement. The technique of treating a wicking mesh layer with surfactants to transport a fluid to an assay sensor is known in the art. See, for example, U.S. Pat. No. 5,271,895 to McCroskey et al. Other examples of known uses of surfactant treated layers are disclosed in U.S. Pat. Nos. 3,992,158 to Przybylowicz et al., 4,050,898 to Goffe, deceased et al., 3,912,457 to Ogawa et al., 4,053,381 to Hamblen et al., 4,774,192 to Terminiello et al., and 4,839,296 to Kennedy et al. Each of the foregoing patents is incorporated by reference in its entirety for all purposes.

Still further, the sensor 120 may be a type that has modified surface tension properties achieved by treatment with a surfactant compound. Such sensors are well known in the art, and include assay pads or strips manufactured and distributed by Medisense, Boehringer Mannheim, Kyoto Dai-ichi (KDK), Miles-Bayer, and Lifescan. Specifically, by way of example only, and not by limitation, electrochemical and calorimetric strips made by Medisense, Boehringer Mannheim, Miles have proven suitable. Likewise, electrochemical strips made by KDK and the colorimetric strip made by Lifescan are also suitable. A specific example of a sensor that is treated with a surfactant is the Elite strip manufactured and sold by Miles-Bayer.

In practice, the sensor used in the integrated device according to the present is smaller in size than the assay strips traditionally used in blood-glucose monitoring systems.

Examples of thickness dimensions for the various components of the integrated device are as follows.

| Element | Thickness (Microns) |
|---|---|
| Optical Window | 20-1000 |
| Sensor & Electrodes | 5-200 |
| Mesh | 20-400 |
| Photothermal Layer | 20-100 |
| Sonic Coupling Portion of Substrate | 100-1000 |
| Complete Assembly | 160-2600 |

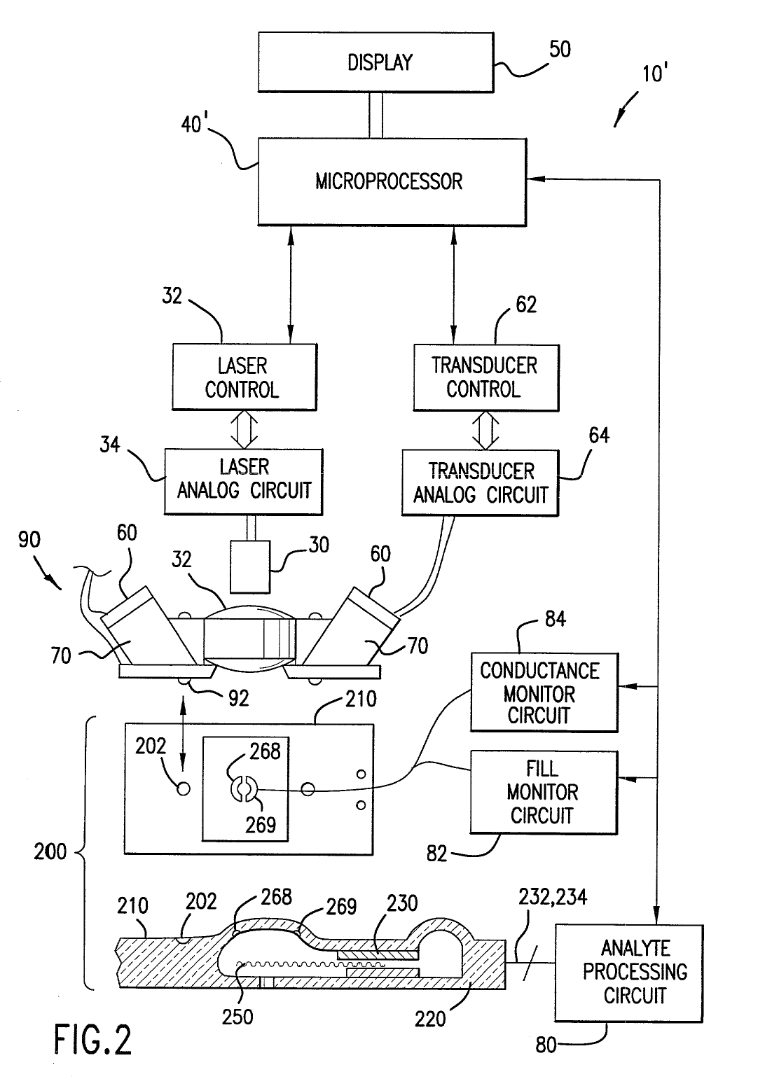

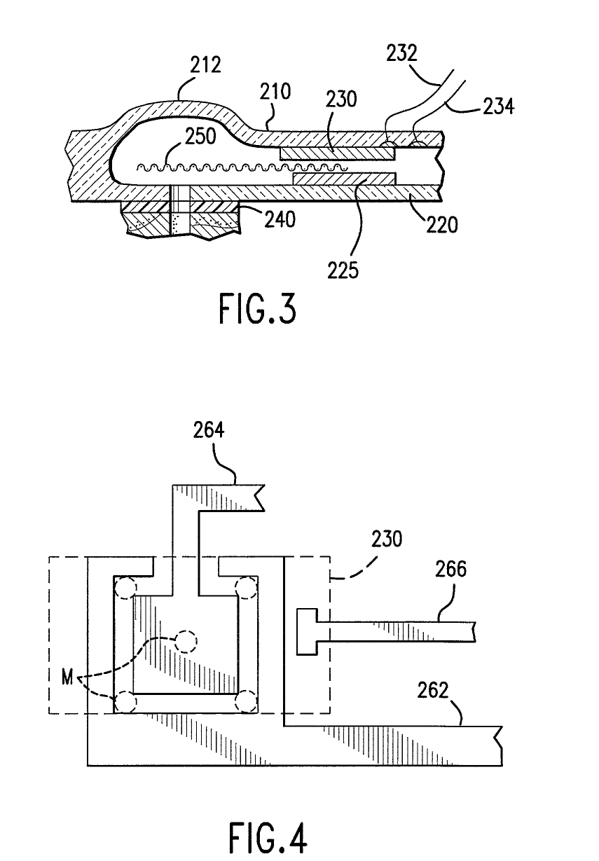

Turning to FIGS. 2-4, an integrated device 200 according to another embodiment is shown. The integrated device 200 is shown as part of an analyte assay system that is similar to system 10′ shown in FIG. 1, but with additional features. The integrated device 200, the details of which are best shown in FIGS. 3 and 4, comprises a top layer 210, a bottom layer 220, and a sensor 230. The top layer 210 may be integral with the bottom layer 220. The top layer 210 has an optically transparent window portion 212. The sensor 230 is disposed between the top layer 210 and the bottom layer 220. A layer of photothermal material 240 is either applied to the tissue, or is formed on or integrally with the bottom layer 220. Electrode leads 232 connect to the sensor 230. Electrical connections to the integrated device 200 are preferably made with a position-invariant type of contact allowing for easy installation and removal of the integrated device 200 from a electrochemical sensor meter and/or optical energy source. FIG. 9 illustrates an embodiment that achieves this in a concentric configuration.

An optional mesh layer 250 is provided in the integrated device to direct collected biological fluid to the sensor. The mesh layer 250 may be treated with a surfactant compound as explained above in conjunction with FIG. 1.

To assist in defining the exact volume of fluid presented to the assay chamber and the sensor 230 a spacer element 225 is placed between the bottom layer 220 and the top layer 210. The spacer element 225 specifies the volume of fluid within the active area of the sensor 230. Specifically, if the aspect ratio of the height (H) of the spacer 225, to the width (W) of the roughly square or circular sensor 230 is less than about 0.1, then for all practical purposes the useable volume of fluid presented to the sensor 230 is defined by H*W*W.

For some embodiments of the present invention, it may be useful to form the micropores some lateral distance away from the sensor 230. In such a case, a surface tension driven or capillary feed channel provides an efficient and volumetrically compact method for conducting the flow of the sampled fluid from the micropore to the sensor 230. This channel may be optionally filled with a mesh layer 250 similar to that traditionally used in a number of existing glucose monitoring strips manufactured by both Boehringer-Mannheim and Medisense as well as others in the industry. As described above, both the capillary channel and the optional mesh layer 250 may be treated with a surfactant compound to enhance the fluid conductivity along this channel.

As shown in FIG. 4, there are two electrodes 262 and 264 that extend in and around the sensor 230, as is well known in the art. The electrode lead 232 connects to electrode 262 and electrode lead 234 connects to electrode 264. Electrodes 262 and 264 are the sense electrodes. Electrodes 268 and 269 shown best in FIG. 2, are the fill electrodes and are connected to the conductance monitor circuit 84 and to the fill monitor circuit 82.

Alternatively, one of the fill electrodes 268 and 269 could be shared with one of the sense electrodes 262 and 264. For example, if a third electrode 266 were strategically placed to the right of electrode 262, and the fill direction was defined to be from the left, then when conductance was sensed between electrodes 262 and 266, this would indicate that the sensor was fully wetted and the reading process can be initiated via the sense electrodes 262 and 262. Similarly, if both a conductance monitor circuit 84 and a fill monitor circuit 82 are incorporated as well as an electrochemical sense system, typically a common anode or common cathode design could be used as one leg of each of these circuits to reduce the total number of electrical traces necessary to be run into the integrated device.

In some forms of the integrated device 200, the conductance monitor circuit 84 is essentially the same as the fill monitor circuit 82, but the conductance monitor is responsive to the very first signs of a fluid sample in the integrated device. This can be used to control the poration process and shut it off in a closed loop fashion as soon as an active pore has been formed as determined by the ability of the pore to source fluid. However, the fill monitor circuit 82 is responsive to determining that the sensor 230 has been sufficiently wetted with a fluid sample in order to begin the measurement of the sensor 230 by the analyte processing circuit 80. The electrode leads 232 and 234 connect the sense electrodes 262 and 264 to the analyte processing circuit.

A microprocessor control circuit 40′ is provided, and is connected to the analyte processing circuit 80 and fill monitor circuit 82. The microprocessor control circuit 40′ is programmed to control the interaction with the integrated device 200. The microprocessor control circuit 40′ generates signals to display analyte measurements and other information on the display 50. In addition, the microprocessor control circuit 40′ controls the application of optical energy and other parameters to the integrated device 200 to effect the microporation and harvesting process. For example, if after the harvesting cycle has begun and some preset amount of time has elapsed during which the fill circuit has not detected a sufficient amount of fluid delivered to the sensor, a suitable error message might be displayed and the user prompted to reinitialize the system, install a new integrated device 200 and try again at a fresh site on the tissue surface. As a further enhancement, a temperature measurement may be obtained from the site and/or sensor in order to correct for temperature sensitivities in the measurements obtained by the sensor.

To this end, the system 10′ includes a laser control circuit 32, a laser analog circuit 34, a transducer control circuit 62 and a transducer analog circuit 64. Alternatively, both the digital control circuitry and analog output stages could be combined into a single circuit or even fabricated as a mixed mode application specific integrated circuit (ASIC). With an ASIC implementation it would also be possible to incorporate all of the master controller microprocessor circuitry, all of the display drivers circuitry, and any other input-output circuitry all on the single ASIC chip, yielding a much simpler, potentially lower cost and more reliable system. The following discussions of the particular functions of each portion of the control and driver circuitry apply equally to either the discrete implementation, a partially integrated ASIC version or a wholly integrated ASIC version.

The laser control circuit 32 is responsive to commands from the microprocessor control circuit 40′ to generate analog signals that are processed by the laser analog circuit 34 to drive the optical source 30. Similarly, the transducer control circuit 62 is responsive to commands from the microprocessor control circuit 40′ to generate analog signals that are processed by the transducer analog circuit 64 to drive the sonic transducer 60.

An interface assembly 90 supports the sonic transducer 60, beam former 70 and focusing lens 32. The interface assembly further comprises an alignment member 92 that mates with an alignment indentation or key 202 on the substrate layer 210 of the integrated device 200. This assures that the optical energy from the source 30 will be properly focused on the integrated device 200, and that sonic energy will be properly coupled through the integrated device 200 to the tissue. These alignment features also ensure that a proper reference is achieved between the micropore formed and the fluid sample collected, and further facilitates proper wetting of the sensor 230 inside the integrated device.

The operation of the system 10′ and integrated device 200 is as follows. Once the integrated device 200 is in position on the surface of the tissue, the microprocessor control circuit 40′ activates the source 30. The source 30 may be a pulsed laser diode, and the microprocessor control circuit 40′ will activate it within a predetermined energy range. The optical energy is focused onto the tissue surface through the substrate top layer 210 and onto the photothermal material 240. The photothermal material 240 is responsive to the optical energy to transfer heat to the surface of the tissue to form one or more micropores therein. As shown in FIG. 4, the one or more micropores M are shown as being made around the periphery or in the middle of the sensor 230. In tests conducted, it was observed that a series of pores placed on one side of the assay pad, under a clear cover layer, effectively formed a capillary feed channel into the area of the assay pad and achieved a uniform wetting of the assay pad as the fluid front swept across it, wetting it without bubbles. Placing the fill sensor electrodes on the side opposite from this fill direction would generally assure that when the fill indicator was tripped, the assay pad could be used to correctly assay the fluid.

The microprocessor 40′ may also control the application of sonic energy. The application of optical energy and/or sonic energy continues until the conductance monitor circuit 84 senses the presence of some amount of biological fluid in the integrated device 200. When the conductance monitor circuit 84 detects the presence of biological fluid in the integrated device, the optical source 30 is deactivated. However, the microprocessor 200 may continue the delivery of sonic energy until the fill monitor circuit 82 detects that the integrated device 200 has collected sufficient biological fluid to make an accurate assay measurement, or until some maximum time period expires. Once this occurs, the biological fluid will contact the sensor 230 and an analyte measurement can be made by the analyte measurement circuit 80, which information is coupled to the microprocessor control circuit 40′ for display on the display 50. The optional wicking mesh layer 250 (optionally treated with a surfactant compound) will assist in directing the biological fluid to the sensor.

Another way to direct the biological fluid to the sensor 230 is by applying a mechanical force downward on the integrated device 200, as shown in FIG. 5. A cam or roller mechanism 280 is applied to the top of the integrated device to form an indent in the integrated device 200 along the top of the layer forming the upper boundary of a capillary channel between the top layer 210 and bottom layer 220. By letting the channel fill to some degree optionally until a fill sensor indicated a sufficient amount has been collected, the cam mechanism 280 is applied in a “squeegee” type action, moving the fluid sample down this channel to the sensor 230. This allows a positive, rapid delivery of fluid to the sensor with a minimum amount of fluid sample.

Furthermore, the application of negative pressure may be used to harvest the biological fluid into the integrated device 200. It has been shown in clinical studies that a small level of pressure reduction, even as little as ¼ ATM, can produce a steady outflow of interstitial fluid from the micropores. The flux rate under vacuum appears to obey an essentially linear relationship with pressure, as the pressure is reduced to 1 ATM, however, optimal values seem to be more in the ¼ to ¾ ATM range due to effects on the surrounding tissue and potential heating of the fluid sample.

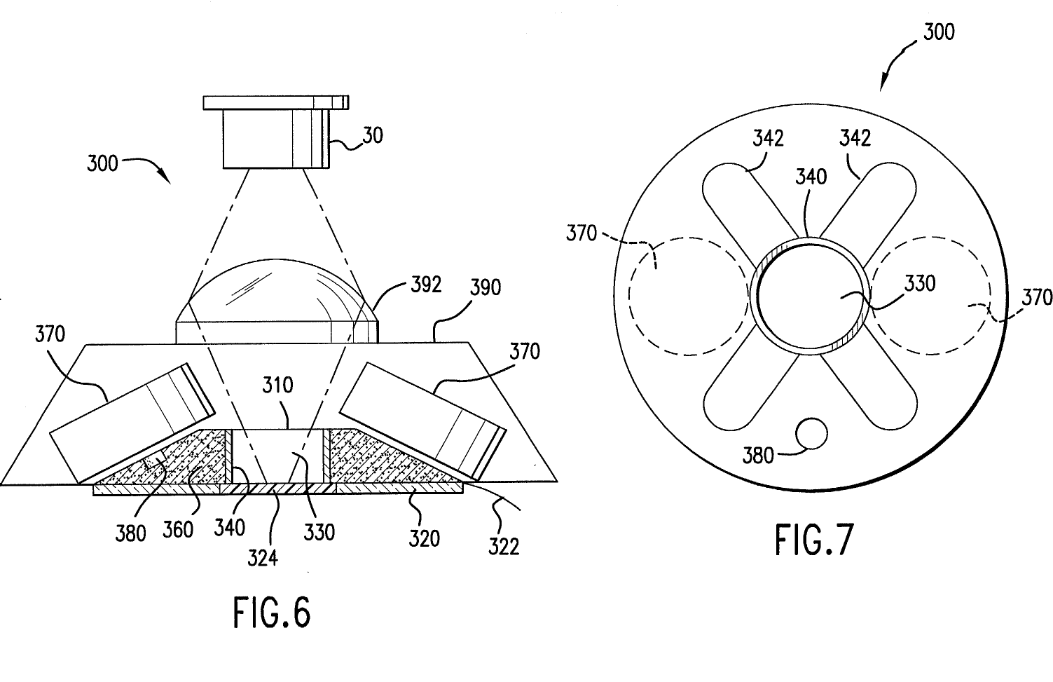

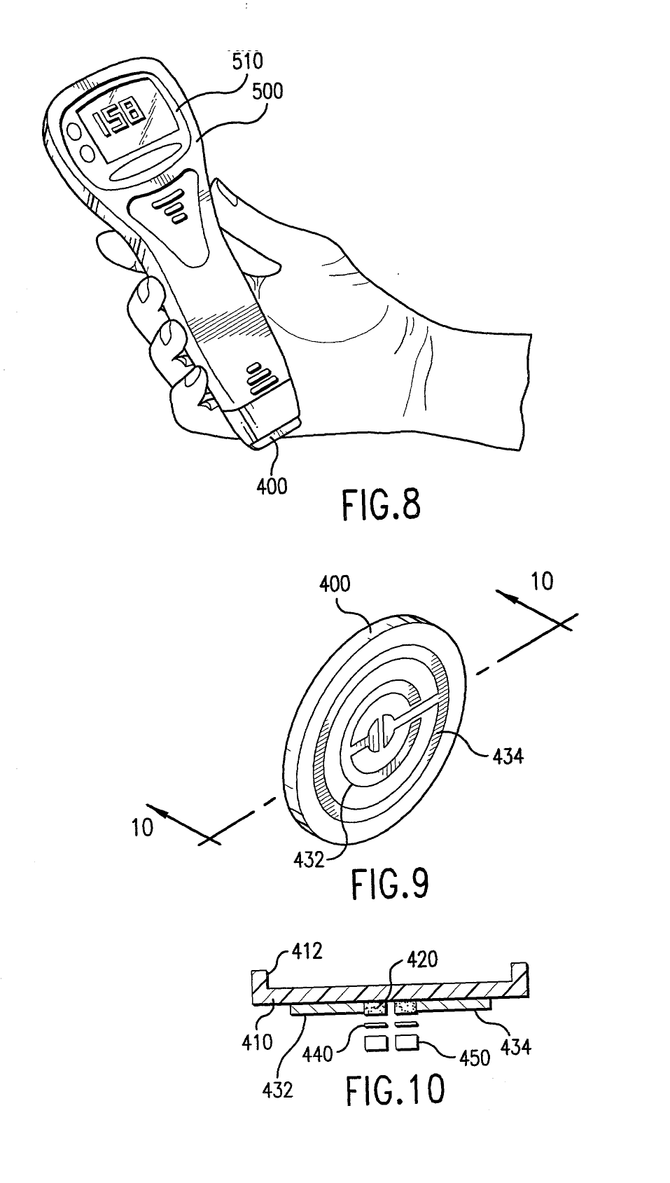

Turning to FIGS. 6-7, an integrated device 300 according to another embodiment is described. The integrated device 300 has a different configuration than integrated devices 100 and 200 described above. Integrated device 300 has a trapezoidal cross-section and comprises an optically transparent top membrane 310 and a bottom layer 320. A removable membrane 322 may be provided that covers the bottom layer 320 until the integrated device 300 is to be positioned on tissue for use. At least a portion of the bottom layer 320 may be treated with a photothermal material 324. Optionally, the membrane 322 is not removable and comprises a thin film of an optically absorbent photothermal material designed to vaporize during the microporation process. According to still another alternative, the tissue itself may be treated with photothermal material before the integrated device 300 is positioned on the tissue. The bottom layer 320 would be made of optically transparent material that vaporizes in the presence of the thermal energy created during poration, or this portion of the bottom layer may be removable just prior to applying the integrated device to the pre-treated area of the tissue surface.

Further, at least a portion of the bottom layer may be treated with an adhesive so that the integrated device 300 is secured to the surface of the tissue. The adhesive serves several functions. First, it preserves proper registration of the openings created in the integrated device with the openings created in the tissue to collect the fluid sample. Second, it allows for attachment of the device to the tissue so that an individual can operate it hands-free. Third, the adhesive will form a vacuum seal between the bottom layer of the integrated device and the tissue surface, thereby preventing biological fluid from passing beneath the integrated device uncollected. The vacuum seal also facilitates the application of negative pressure to the harvesting site.

The integrated device 300 comprises a chamber 330 to collect biological fluid in the center portion of the device. The chamber 330 is cylindrical in shape as shown in FIGS. 6 and 7. A sensor 340 is disposed on at least a portion of the inner wall of the chamber 340. Although FIGS. 6 and 7 show a cylindrical chamber, any other shape convenient to a particular application may be used for the chamber. For example, if the integrated device is manufactured in large quantities, a flat walled shape, such as a triangle, square or pentagon, in which the active sensor pad is placed on one of these walls.

As best shown in FIG. 7, a typical electrical wiring diagram is shown to support most electrochemical sensors used to date. There are an anode 342 and cathode 344 that connect to the sensor 340 each of which extends outward from the center of the integrated device 300. In addition, a reference electrode 352 and a sense electrode 354 are provided that connect to the sensor 340.

Optionally, the integrated device 300 may include additional electrodes to support the “fill” and “conductance” features described above, or even if the electrochemical assay system used requires additional electrical interfaces to function optimally. The reference electrode is useful for many electrochemical assays to provide a self calibrating feature wherein the actual assay reaction can be read as more of a difference measurement across a balanced impedance bridge. The sense electrode could be the assay output, which is similar to the “fill” or a “conductance” signal described above. Although dedicated anode and cathode terminals are shown, it is common practice to use one or the other of these as the sense electrode and share the other one with the reference electrode. The electrode configuration shown in FIGS. 6 and 7 indicates that the integrated device 300 supports the use of many of electrode configurations.

Disposed around the chamber 330 between the top membrane 310 and the bottom layer 320 is an acoustic lens 360 formed of material suitable for coupling sonic energy. For example, the acoustic lens 360 is formed of silicone material molded into a shape suitable for being disposed inside the integrated device 330. A lens material of a suitable durometer value will ensure sufficient sonic coupling to the tissue beneath, and also achieves pneumatic sealing if suction or negative pressure is used in the harvesting process. Sonic transducers 370 are positioned on the lateral surfaces of the integrated device to deliver sonic energy to the tissue through the acoustic lens 360. In order to facilitate alignment of the integrated device 300 with the remaining components of an assay system (similar to that shown in FIGS. 1 and 2), there is a reference hole 380 provided on the surface of the integrated device 300. The integrated device 300 may be contained within a housing 390 that includes an optical focusing lens 392 molded therein to focus optical energy from source 30 onto the photothermal material 324.

The operation of the integrated device 300 is similar to the descriptions above of integrated devices 100 and 200. However, the biological fluid harvested by the integrated device 300 is collected in the chamber 330 and contacts the sensor 340 placed on the side walls of the chamber 330, rather than a planar sensor shown in the previous embodiments.

Turning to FIGS. 8-10, an integrated device 400 according to a third specific embodiment will be described. The integrated device 400 is designed for use with a hand-held unit 500 that processes assay measurements obtained by the integrated device 400 and displays the measurements on a display 510.

The integrated device 400 is a disc-shaped member that supports the components for fluid harvesting and assay measurement. The integrated device 400 comprises a substrate member 410 cond in a circular shape and having flanges 412 that snap fit onto a bottom portion of the hand-held unit 500. A sensor 420 is positioned at a central location on one surface of the integrated device 400. First and second electrodes (anode and cathode) 432 and 434 extend concentrically on the bottom surface of the substrate 410 and make connection from opposite sides to the sensor 420. An optional mesh 440 may be disposed over the sensor 420. The mesh 440 may be treated with a suitable surfactant compound described above. A layer of photothermal material 450 is disposed over the opening placed on the bottom of the integrated device to allow the harvested fluid to wet the sensor 420, or the tissue itself is treated with photothermal material 450.

The integrated device 400 is operated by attachment to the hand-held unit 500 and positioned on the surface of the tissue to be microporated. The hand-held unit contains the optical source to focus optical energy onto the photothermal layer 450 to form one or more micropores in the tissue. Biological fluid from the tissue makes contact with the sensor 420 (preferably with the assistance of the mesh layer 440). The hand-held unit 500 includes processing circuitry that electrically couples to the electrodes 432 and 434 to obtain an assay measurement from the sensor 420. The hand-held unit 500 is activated by engaging the integrated device 400 against the tissue surface with sufficient pressure together with pressing an activation button on the hand-held unit 500.

In the previous embodiments of the integrated device, the poration process is based on the application of optical energy to an absorber target which in turn heats up sufficiently to conductively deliver enough thermal energy to the skin to ultimately cause the desired thermally induced microporation. An alternative approach to delivering this heat energy to the poration sites involves the placement of an electrically heated probe directly at the poration site. The temperature of the electrically heated probe is modulated as needed to effect the microporation process.

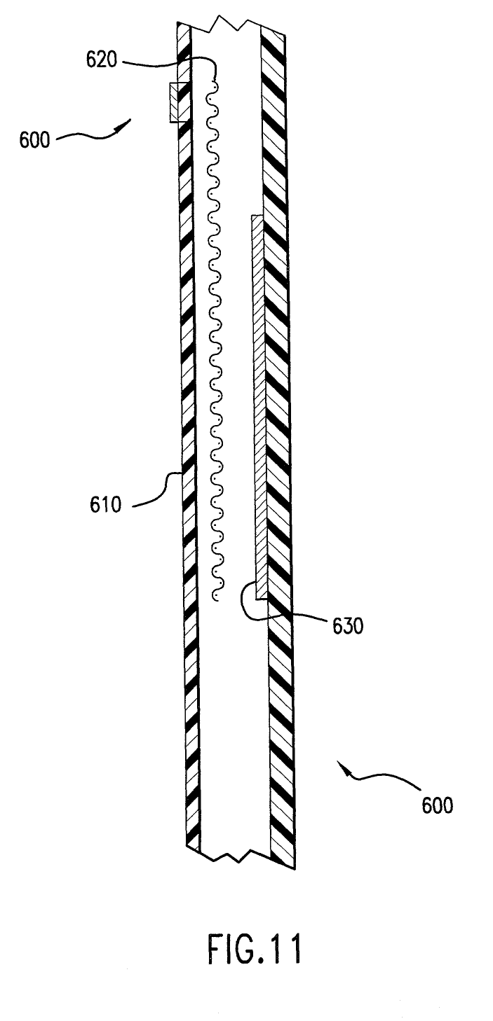

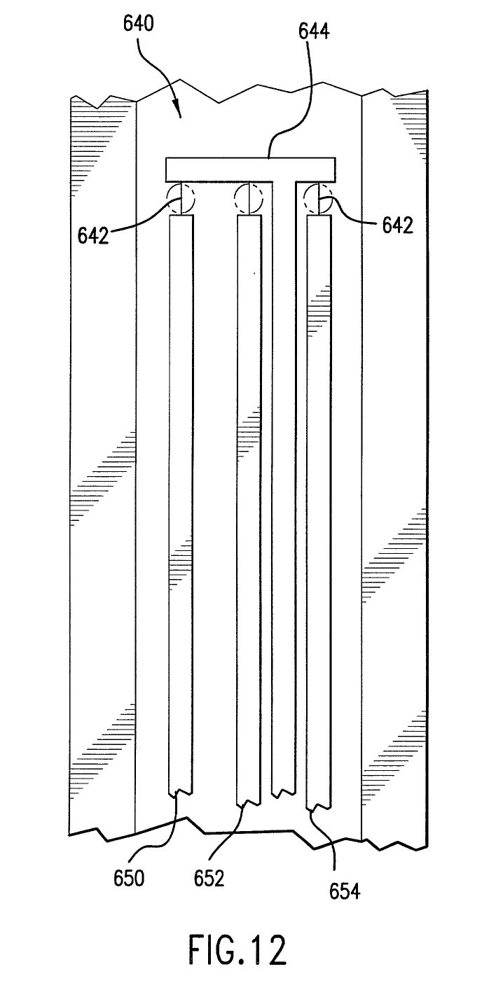

A schematic representation of an integrated device 600 employing an electrically heated probe (heat conducting element) is shown in FIGS. 11 and 12. The integrated device 600 comprises a layer 610, an optional mesh layer 620, and a sensor 630, which in this example, is a calorimetric sensor. It should be understood, however, that this same concept could easily be modified to employ the electrochemical biosensor. Moreover, as described in the foregoing, many of the aspects of the assay/fluid management systems of the device are optional, such as the use of the mesh layer 620, surfactant treated portions of the fluid management chamber, optically transparent windows in the layers to allow the reading of a colorimetric assay, methods for applying sonic energy, vacuum or negative pressure, mechanical manipulation, etc.

In the integrated device 600, there is provided at least one electrically heated probe 640. The types of electrically heated probes that are suitable are disclosed in the aforementioned published PCT application, WO 9707734, which is incorporated herein by reference.

As shown in more detail in FIG. 12, the electrically heated probe 640 comprises an electrically conductive element or wire 642 provided on the bottom surface of the layer 12. Three electrically conductive elements 640 are shown as an example, though any number of them may be provided. An electrical conductor 644 extends the length of the layer 610 and terminates in a “T” that extends laterally across one end of the layer 610. Three other electrical conductors, 650, 652 and 654 extend the length of the layer 610 and terminate at a plurality of points near the termination of conductor 644. The three elements 640 are connected to conductor 642 and respectively to conductors 650, 652 and 654.

The electrical conductors 644, 650, 652 and 654 required to activate the elements 640 (also called poration elements hereinafter) can be made through the same type of connectors used to interface to the electrical output electrochemical biosensor. Each poration element 640 can be activated individually through the appropriate selection and energization of the conductors 650, 652 and 654. It may be advantageous to excite all poration elements 640 simultaneously, thereby enabling either a series or parallel wiring design, reducing the number of interconnections to the disposable poration system and facilitating a more rapid poration process. If only one element 640 is provided, then at least two conductors are provided for supplying electric current through the heatable element. One of these conductors may be shared with the assay sensor/fill/conductance circuitry as a common anode or cathode, thereby necessitating the additional of only one electrical connection to the integrated device.

These electrically activated thermal poration elements could be installed on a conventionally manufactured assay strip as an additional post-processing step. Preferably, the conductors 644, 650, 652 and 654 are embedded within the tissue-contacting layer so as not to be exposed on the bottom surface thereof, but to enable sufficient electrical connection to the one or more heated elements 640.

Each of the elements 640 functions as a solid thermal probe and is electrically heated so that the temperature of the tissue, if skin, is raised to a temperature greater than 123° C. For example, each element comprises a 100 to 500 micron long 50 micron diameter tungsten wire. These tungsten wires are typically laid flat against some form of backing (such as the tissue-contacting layer 12) which naturally limits the depth of penetration of the wire into the tissue (by virtue of the diameter of the wire). The temperature of the wire may be modulated according to the techniques disclosed in the aforementioned PCT publication.

The inlet ports to the fluid management chamber of the integrated device 600 may be small holes in the tissue-contacting layer across which the wires 640 extend. Alternatively, a meltable or vaporizable membrane is placed above the wires 640. When energized, the wires melt a hole in this membrane, creating an inlet port to the fluid management chamber at each location of the wires 640.

A system can be designed wherein the electrically heated poration elements 640 are contained in a separate component or device, which may be reusable. These elements would be replaced when it is detected that they are worn sufficiently to require replacement, or routinely, such as on a weekly basis, similar to a diabetic subject's replacement of a lancet tip in a fingertip lancing blood-drawing device.

In all of the foregoing embodiments of the integrated device and assay system according to the present invention, the type of optical energy source may be any of those described in the aforementioned PCT application WO 9707734. Likewise, the types of substances used for the photothermal material are disclosed in PCT publication WO 9707734 and the aforementioned U.S. provisional application, which is also incorporated herein by reference.

In the foregoing embodiments of the integrated device, the sensor used to react with the harvested biological fluid and measure a characteristic of the fluid may be an electrochemical biosensor comprised of a layer or layers of chemicals capable of reacting with an analyte in a collected biological fluid to produce a measurable electrical response (as specifically shown in some of the foregoing embodiments). U.S. Pat. Nos. 4,545,382 and 4,711,245 describe detecting layers capable of generating a measurable electrical signal in response to glucose in blood. The electrical signals are measured by a measuring circuit (such as the one shown in FIGS. 1 and 2 at reference numerals 20 and 80, respectively) obtained by electrical leads connected to electrodes in or around the active area of the biosensor. Alternatively, the sensor may be a calorimeter sensor, a fluorescent intensity-based sensor or a fluorescent life-time based sensor. Examples of electrochemical biosensors that are suitable for use in the integrated device are those manufactured by Medisense, Boehringer Manheim, KDK, etc.

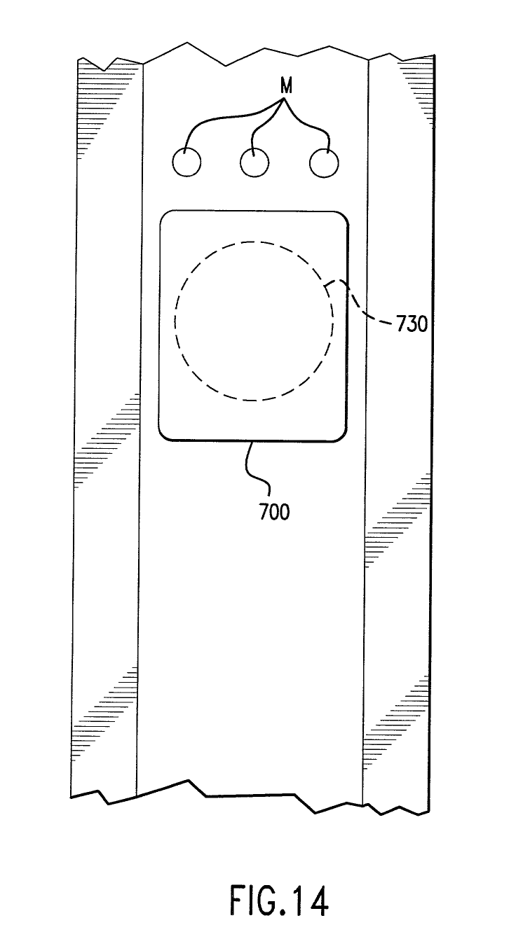

If a colorimeter sensor, a fluorescent intensity-based sensor or a fluorescent life-time based sensor is used, the sensor is “read” by an arrangement shown in FIGS. 13 and 14. The integrated device shown in FIG. 13 may be any one of those described in the foregoing embodiments. The sensor to be optically read is shown at reference numeral 700. The sensor 700 is held in sufficient registration to enable the optical field of view 730 of an optical meter 720 to be placed nominally in the center of the region of the colorimetric sensor wetted by biological fluid. The field of view of the optical meter 720 is optically cropped such that it conservatively under fills the area known to be wetted by the fluid sample. This reduces both the precision required during manufacture of the integrated device and the degree of initial and maintained registration of the integrated device on the meter and the individual, thus reducing cost and increasing reliability. In addition, this reduces the actual volume of biological fluid required to produce an accurate reading of the amount of the selected analyte present in the biological fluid. Colorimetric sensor technology for measuring glucose concentration is well known in the art. Furthermore, examples of fluorescent based sensor technology are disclosed in U.S. Pat. Nos. 5,660,991; 5,631,169; 5,624,847; 5,504,337; 5,485,530 and 5,281,825, all to Lakowicz et al.

Specifically, it is a standard in the field of disposable assay strips to completely wet an area of the reagent treated portion of the assay strip much larger, typically 5 to 10 times larger, than the total area actually read by the meter. This practice allows relaxation of manufacturing tolerances in many parts of the system. This is also a common feature in the “fingerstick” blood-based glucose monitoring systems due to the physical difficulty of the user placing a smaller sample only on the actual target spot as well as the need for most whole blood-based systems to separate the corpuscular components from the serum. By incorporating the automatic registration of the micropores M with the sensor 700 through the design of the device, the assay process can be conducted accurately with a much smaller sample of the fluid than the typical fluid based disposable assay technology currently available.

If the assay technique used in connection with the integrated device is based on a fluorescent intensity technology, the colorimetric sensor 700 is treated with a probe fluorophore. A reaction between a probe fluorophore and the selected analyte produces a predictable change in the fluorescent intensity of the probe molecules when excited with a particular optical wavelength such that the subsequent fluorescence is detected at a selected longer wavelength. Optionally, the fluorescent probe is selected such that it can emit in two different wavelength bands, wherein the intensity of energy in only one of the bands is predictably modified by the varying concentration of the selected analyte. A ratiometric processing of the two different fluorescent intensities can be employed, thereby simplifying the calibration of the reading and allows for self-adjustment for different amounts or areas of the colorimetric sensor wetted with the biological fluid. Moreover, the fluorescent interrogation field of view may be defined by the intersection of the incident excitation light and the look field of the fluorescent receive channels.

Further still, the assay technique used in conjunction with the integrated device may be based on a fluorescent lifetime based assay technology. In this case, a reaction between a probe fluorophore, with which the calorimetric sensor 700 is treated, and the selected analyte produces a predictable change in the fluorescent lifetime of the probe molecules when excited with a particular wavelength. The subsequent fluorescent lifetime is detected at a selected longer wavelength. The detection of the fluorescent lifetime may be accomplished by either measuring directly the decay of the fluorescence in response to a known pulse shape of excitation light, or by measuring the phase shift and modulation depth of the fluorescent signal in response to the excitation of the sensor by a periodic modulated light source at the appropriate excitation wavelength. By basing the quantification of the analyte on a time resolved measurement, much of the difficulty associated with the calibration of an absolute intensity based measurement is overcome. Also, the signal-to-noise aspects of such a system are easily optimized. For example, in a phase detection system, it is routine to integrate for a sufficient period of time in order to resolve the phase to any level needed. Consequently, very small amounts of the probe molecule and biological fluid are required to achieve the desired level of quantification of the selected analyte, yielding additional benefits in the potential reduction of the required biological fluid sample volumes to the levels of only a few hundred nanoliters.

The integrated device according to the present invention may be used in a continuous monitoring system. The system could be integrated with other devices, including an insulin pump. The continuous monitoring system would provide a real-time feedback to achieve a closed loop artificial pancreas system without requiring an implant. The integrated device would be connected to a “smart” insulin pump that would respond initiate a glucose measurement (or one would be initiated on demand by the patient) and would administer an appropriate amount of insulin depending on the glucose measurement obtained.

In all of the foregoing embodiments on the integrated device, each has a fluid management chamber designed to direct the biological fluid collected to the sensor. The surfaces of the fluid management chamber may be selectively treated with chemical substances, such as a wicking agent, or a surfactant to induce the migration of fluid in a particular direction, i.e., to the sensor. Alternatively, certain portions of the surfaces of the layers in the fluid management chamber, such as the tissue-contacting layer, may be treated with a hydrophobic compounds or substances to direct the biological fluid away from a selected region or regions where it is not desired for the biological fluid to migrate and to direct the biological fluid toward the sensor. In the continuous monitoring embodiments described above, additional fluid management considerations may have to be given to deal with the “waste” fluid which would be created as a fresh fluid sample where harvested and moved into the sensor area. One solution for this waste fluid is to merely expand the overflow region provided in many of the current assay strip designs discussed and referenced above, to a size large enough to act as a fluid sink for this used fluid sample. This expanded overflow region also addresses the desire to keep these fluid samples contained within the integrated device element to maintain control over the ultimate disposal of the biological sample such that it can be handled with appropriate caution.

Furthermore, by designing the integrated device in such a manner that the biological fluid management is handled with minimal dead space outside of the active region of the sensor, a system can be built which uses very small samples of biological fluid to obtain an accurate assay of a selected analyte. Tests have been conducted on commercially available systems using glucose sensing amperometric biosensors that incorporated all of these features and it was found that the glucose concentration in a sample of biological fluid smaller than ⅓ of a microliter could be quantified, by modifying commercially available glucose test strips. One of the additional advantages gained by using interstitial fluid as the fluid sample for the assay system is the almost total lack of red blood cells in the sample. Most commercial blood-strip based assay systems utilize some means of separating the corpuscular component from a whole blood sample prior to the application of the fluid sample to the assay element. In many cases, this process is performed by the use of some sort of wicking mesh designed to trap the blood cells and let only the serum move through to the assay area. These filtering approaches can use up as much as ⅘ of the original sample volume in the process. By using interstitial fluid, this step is no longer needed. In other words, a typical sample size of 3 to 10 microliters is normally required for a blood based glucose monitoring disposable assay strip design whereas by utilizing the ability to place an unfiltered interstitial fluid sample directly on the active reagent treated portion of an assay system, it has been demonstrated that quantitative readings of a selected analyte can be obtained with fluid samples as small as ⅓ μL of interstitial fluid using modified conventional disposable assay strip technologies.

Another example of a closed loop application of the integrated device is to collect and monitor the levels of a particular drug in the blood stream or other fluid, wherein it is desired to maintain the drug level within a defined serum concentration window. In this case, an infusion pump would be controlled to respond, taking into account the total system bandwidth, and deliver small pulses of the therapeutic drug into the subject until the desired set point level had been reached, wherein the pump would then be put on standby until the drug levels dropped below the set point, thus triggering another pulse of the drug. Bandwidth in this context refers to the sum of all delays associated with the infusion of the drug, distribution within the body, diffusion into the fluid reservoirs from which the integrated device collects its fluid sample, and any additional delays in the sampling and processing involved before a change in the reported assay value would occur. Several drugs commonly prescribed could benefit from this sort of tight control over dosage levels such as many of the anti-seizure drugs, pain medications, chemotherapies, etc. In the case of pain medication, an additional control input could be given to the user allowing them to ask for an additional dose in an on-demand fashion to deal with breakthrough pain, and use the closed loop monitoring feature as a final safety to ensure than no toxic overdose levels would occur.

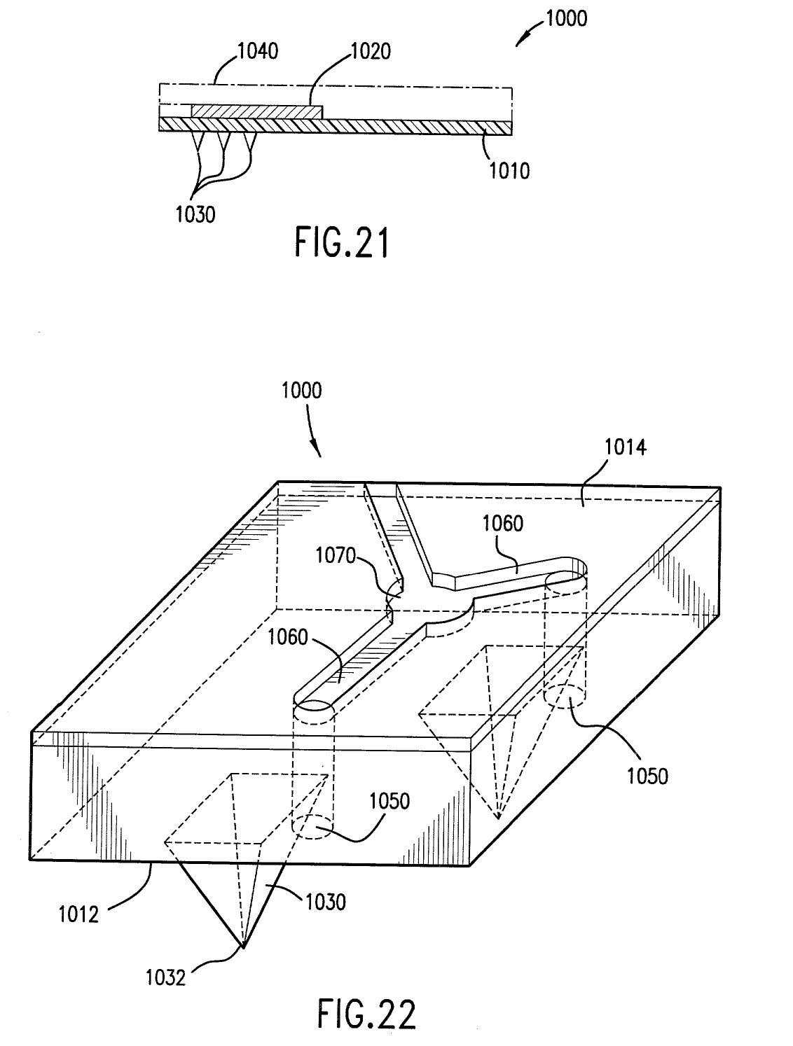

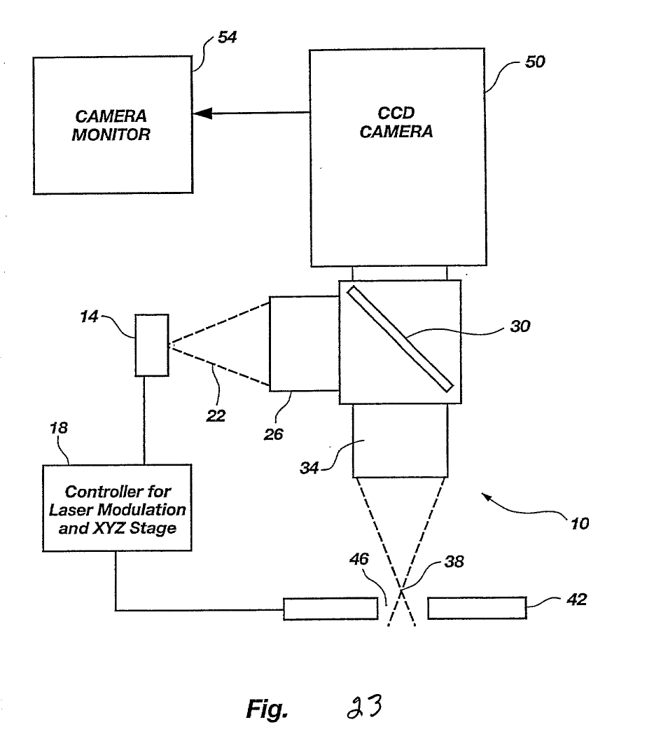

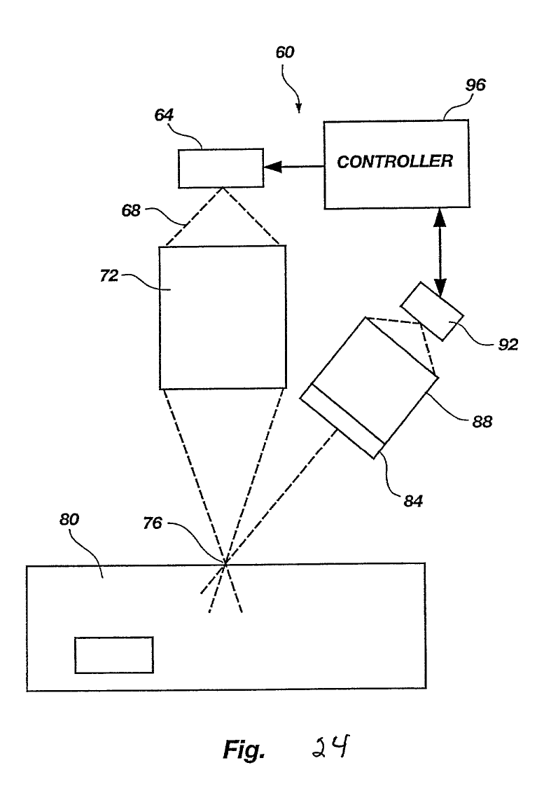

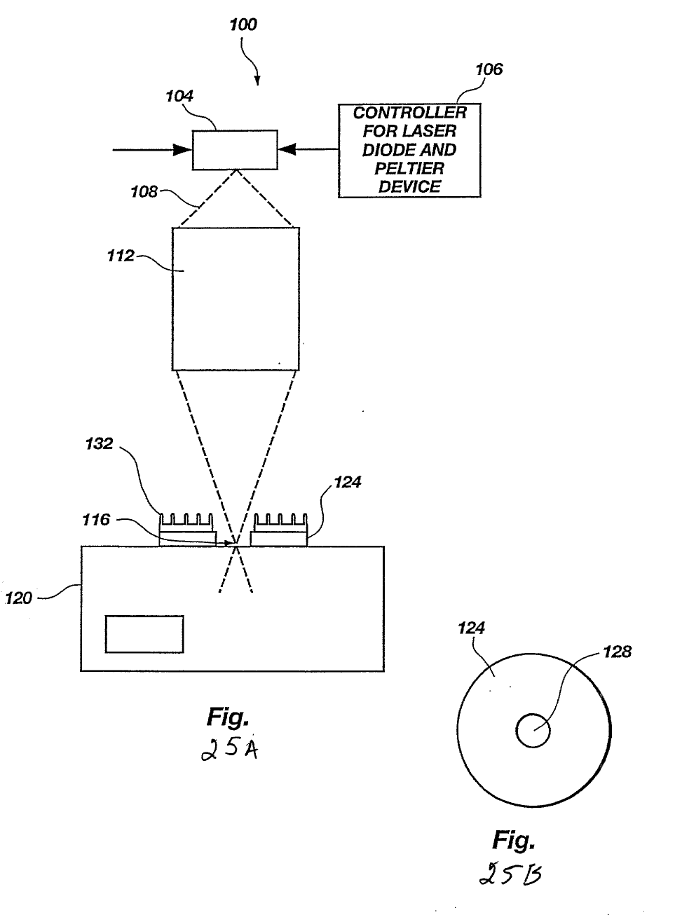

Turning to FIGS. 21 and 22, an integrated device 1000 according to still another embodiment is shown. The integrated device 1000 is similar in many respects to the previous embodiments, but includes one or more mechanical porating elements. Specifically, the integrated device 1000 comprises a substrate layer 1010 and a plurality of mechanical porating elements 1030 protruding from the bottom of the substrate layer 1010. The specific structure and arrangement of the mechanical porating elements are disclosed in commonly assigned published PCT application WO 9800193, referred to above, and which is incorporated herein by reference. The integrated device 1000 further comprises an assay sensor 1020 disposed above the mechanical porating elements 1030. The assay sensor 1020 is any of the sensors described above. An optional additional top layer 1040 may be provided to seal the top surface of the integrated device. If the assay sensor 1020 is a type that is optically read, then the top layer 1040 is optically transparent.

The mechanical porating elements 1030 are puncturing elements very small in size (10 to 50 microns), and are spaced apart from each other. With reference to FIG. 22, each porating element 1030 comprises a sharp point or edge 1032 for puncturing the tissue surface. Depending on the desired depth of the micropores to be created, the height of the porating element 1030 will vary. The porating elements 1030 may be pyramid or wedge shaped, which is easily created by microfabrication techniques, such as microlithography. Other shapes for the porating elements 1030 may be suitable, such as that of micro-lancets or micro-needles.

There are pluralities of holes 1050 extending from the lower side 1012 of the substrate layer 1010 on which the porating elements 1030 are exposed, to the upper side of the substrate layer 1014. Each porating element 1030 is adjacent to and paired with at least one hole for collecting biological fluid that seeps out of the punctured tissue. The holes 1050 are of suitable dimension to permit biological fluid, such as blood or interstitial fluid, to move by capillary action from the lower side 1012 of the substrate layer 1010 to the upper side 1014. The holes 1050 may be interconnected with channels 1060 that are formed on the upper side 1014, and the channels 1060 may intersect at a reservoir 1070. The assay sensor 1020 (not shown in FIG. 22 for simplicity) is then positioned on top of, partially overlapping, or adjacent the reservoir 170, so that it is sufficiently wetted with the biological fluid to make a suitable measurement.

As in the foregoing embodiments, the integrated device 1000 may include surface tension enhancements, such as a wicking mesh (surfactant treated or not) and surfactant treatment of the holes 1050 and channels 1060. The wicking mesh would be positioned to overlie the reservoir 1070 and thereby enhance the transport of the biological fluid to the assay sensor 1020. Furthermore, the integrated device 1000 may be modified to include any of the enhancements discussed below.

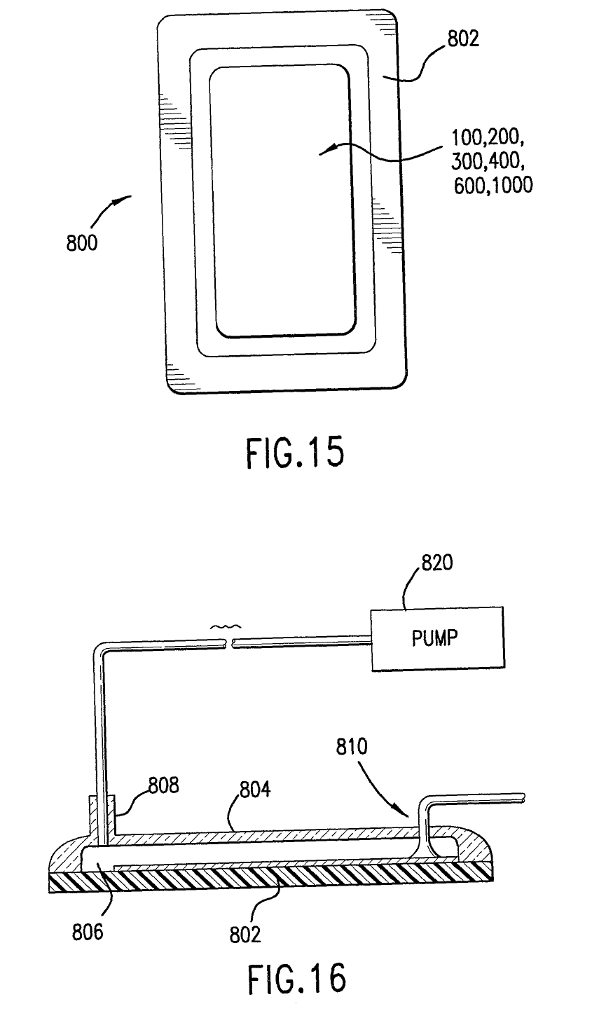

To this end, FIGS. 15 and 16 illustrate the use of a pneumatic seal together with any one of the integrated devices described above. A sealing means in the form of a sealing assembly 800 is provided which comprises a perimeter base 802 that fits around the integrated device (100, 200, 300, 400, 600, 1000), and a top layer 804 that is sealed to the perimeter base 802, and extends above the integrated device. The sealing assembly 800 pneumatically seals around the integrated device to the surface of the tissue. If the integrated device is of the type that requires exposure to optical energy, the top layer 804 is made of optically transparent material. The perimeter base 802 seals to the tissue surface around the integrated device, such as by an adhesive, or a tacky silicone, rubber or plastic element. A sealed chamber 806 is formed in the space between the integrated device and the top layer 804. A vacuum port 808 is provided in the top layer 804 for connection to a means for supplying negative pressure, such as a pump 820 or other source of negative pressure, such as a syringe, a diaphragm or some portion of the chamber which can be flexed outward to increase the volume of the chamber and thereby reduce the pressure within the chamber or the like. In addition, if an integrated device is used that requires connection to an electrode on the sensor and/or probe, this connection is made through a sealed electrical connector 810 in the top layer 804.

The sealed chamber 106 is formed against the surface of the tissue, such as the skin, over the poration site(s). The pressure in the chamber 106 can be reduced to provide a positive pressure gradient from within the body towards the sealed chamber 106 through the micropores to induce the biological fluid to exit the body and enter the integrated device 10 more rapidly.

By maintaining the total internal volume of the chamber 806 as small as possible, only providing the needed clearance for the integrated device, the evaporative losses of the biological fluid can be minimized. Once the humidity inside the chamber 806 reaches a saturation point, no more evaporative losses can occur. These evaporative losses can further be reduced by managing the biological fluid in a manner wherein the exposed surface area of the biological fluid pool that has exited the tissue is kept small. When induced to enter the device, the biological fluid is constrained on all sides other than the port(s) in the fluid management chamber at the microporated site. The side layer or wall of the fluid management chamber opposite these ports could be constructed with one or more very small opening(s) to create a vent allowing the biological fluid to fully fill the fluid management chamber, yet minimize the exposed surface of the biological fluid when the assay area is full, thereby reducing evaporation. The reduction of evaporative losses is more significant when using a vacuum-induced harvesting process because the rarefied atmosphere will accelerate any evaporation process. Experiments have shown that simply keeping the volume of the chamber small, and providing a capillary type channel (comprised of the sensor on one side and a layer on the other with or without the optional wicking mesh therebetween) for the biological fluid to enter upon exiting the body, evaporative losses can consistently be kept under 5% over a 45 second harvesting cycle. A large chamber and an exposed bead of biological fluid on the surface of the skin can allow up to 30% of the biological fluid to evaporate during this same 45 second interval, under the same temperature conditions.

An additional feature of pneumatically sealing the integrated device is that by virtue of its contact with the tissue, these portions of the integrated assay system maintain the mechanical alignment of the micropore(s) in the tissue with the inlet ports of the integrated device.

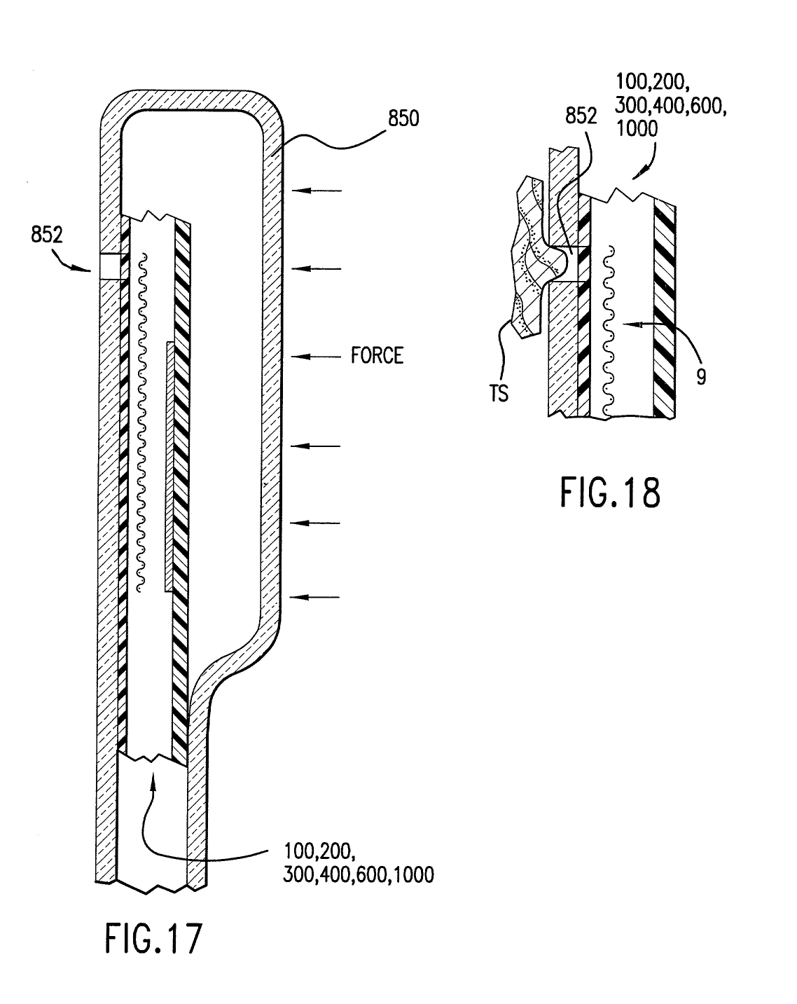

FIGS. 17 and 18 illustrate the use of a mechanical system to apply positive pressure to the integrated device. A mechanical element 850 is provided, having a small opening 852, 2 mm to 4 mm in diameter. The mechanical element 850 permits the integrated device to slide between two opposing surfaces and contains the integrated device. Applying force to the mechanical element 850 presses the integrated device onto the skin at the poration site and thus creates a positive pressure gradient in the biological fluid harvested from the tissue TS, i.e., the skin, forcing it towards the micropores where it can exit the tissue and enter the inlet port(s) of the fluid management chamber of the integrated device (100, 200, 300, 400, 600, 1000). The tissue bulges into the opening 852 as shown in FIG. 18. A close registration is maintained between the inlet ports to the integrated device and the micropores, which have been, or simultaneously will be, formed in the tissue directly beneath these ports. The mechanical device 850 may be optically clear on its top portion to allow for optical thermal ablation and optical reading of the photometric sensor in that form of the integrated device.

The application of mechanically induced pressure may be continuous, modulated as in a sine or triangle wave, or pulsed. The rate and modulation pattern may be optimized to take advantage of the fluidic properties of the skin tissues such as the local permeabilities, and the refill or recovery rates of the tissue once some portion of the biological fluid has been pressed out of it. Clinical experiments have demonstrated that applying a few pounds per square inch of pressure to the skin with a flat plate having a 2 mm to 4 mm diameter hole in it surrounding the micropore(s) rapidly forces biological fluid to exit the pores and pool on the surface of the skin. In addition, the use of the mechanical device may be combined with vacuum to provide an additional biological fluid forcing function, and to possibly assist in the fluid management of the biological fluid as it exits the body. A further benefit of applying firm pressure to the system during the thermal poration process is that this pressure helps ensure a good thermal connection between the heat probe created by the optically heated absorber targets and the skin to be porated.

One significant advantage of these preferred integrated microporation, harvesting, assay system is that the input ports or channels to the assay system are in physical registration or alignment with the micropores on the skin to ensure an efficient transfer of fluid from the micropores to the assay strip. Registration and alignment can be achieved by employing an adhesive or tacky silicone product to temporarily attach the integrated device. Alternatively, registration and alignment can be accomplished by installing the assay strip component within a translation system which, when activated, brings the input ports or channels of the assay strip into close enough proximity to the biological fluid exiting the micropores to cause the directed flow of this biological fluid into the assay strip. This sort of translation can be achieved in a number of ways such as, but not limited to, a small servo motor activated by a controller to move the assay strip into position at the appropriate time; a pneumatically positioned system driven by the same vacuum source described in conjunction with FIGS. 15 and 16; or a system design wherein the flexure of the skin itself under either the vacuum or pressure as described above brings the biological fluid on the surface of the skin into contact with the assay strip. An additional advantage of the translation system in the fluid management portion of the integrated microporation, harvesting, assay system is that it can be designed to supply the entire required fluid sample in a bolus delivery to the assay system, rather than trickling it over some longer period of time. In many cases a bolus delivery of sample fluid enables a more accurate assay to be conducted using standard disposable assay strip design concepts.

Furthermore, by designing the integrated microporation, harvesting, assay system in such a manner that the biological fluid management is handled with minimal dead space outside of the active region of the biosensor, a system can be built which uses very small samples of biological fluid to obtain an accurate assay of a selected analyte. Tests have been conducted on commercially available systems using glucose sensing amperometric biosensors that incorporated all of these features and it was found that the glucose concentration in a sample of biological fluid smaller than ⅓ of a microliter could be quantified, by modifying commercially available glucose test strips. One of the additional advantages gained by using interstitial fluid as the fluid sample for the assay system is the almost total lack of red blood cells in the sample. Most commercial strip based assay systems utilize some means of separating the corpuscular component from a whole blood sample prior to the application of the fluid sample to the assay element. In many cases, this process is performed by the use of some sort of wicking mesh designed to trap the blood cells and let only the serum move through to the assay area. These filtering approaches can use up as much as ⅘ of the original sample volume in the process. By using interstitial fluid, this step is no longer needed. In other words, a typical sample size of 3 to 10 microliters is normally required for a blood based glucose monitoring disposable assay strip design whereas by utilizing the ability to place an unfiltered interstitial fluid sample directly on the active reagent treated portion of an assay system, it has been demonstrated that quantitative readings of a selected analyte can be obtained with fluid samples as small as ⅓ μL of interstitial fluid using modified conventional disposable assay strip technologies.

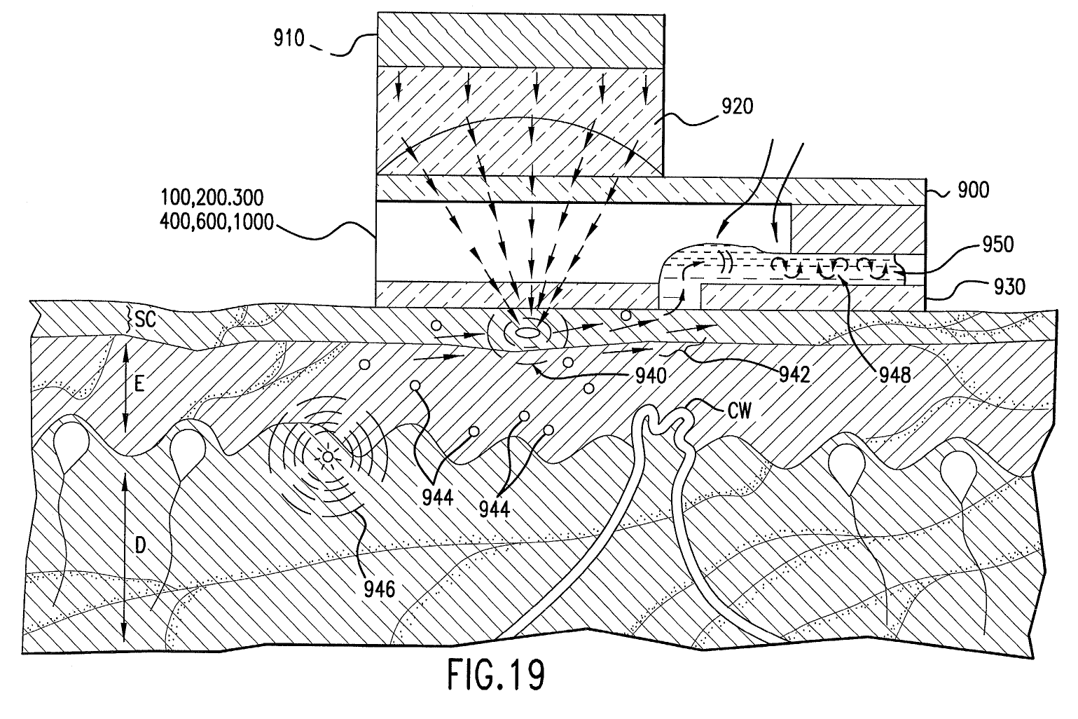

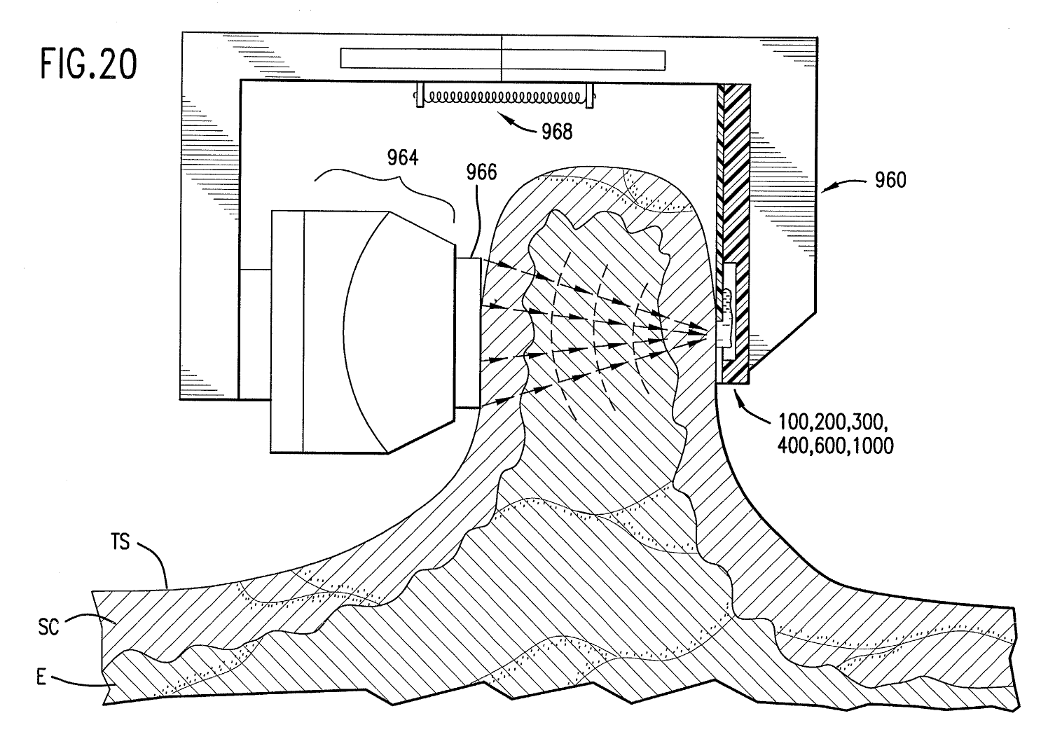

Turning to FIGS. 19 and 20, the use of sonic energy in conjunction with the integrated device will be described. The integrated device can be used in conjunction with a means for coupling sonic energy from a transducer into the system and optionally into the tissues upon which the integrated system is disposed. In particular, experiments have shown that sonic energy in the range of 5 kHz to 30 MHz can be useful to enhance the outflux of biological fluid from a microporated area of skin. Furthermore, the literature on the use of sonic energy supports the extension of the useable frequencies as high as 500 MHz.

The permeation enhancing effect of sonic energy is due to several different mechanisms in the tissue, including but not limited to, the acoustic streaming induced in the fluids within the skin tissues, the directable effects of the sonic radiation pressure which can act directly to push the fluid is a desired direction, the reduction in the viscosity of the fluid itself, the modification of the surface tension effects both within the tissues and at the surface of the micropore, the local heating possible from the absorption of the sonic energy and the body's natural edemic response to this, the opening of microscopic temporary channels in the various membranes and layers within the tissue such as the capillary and vessel walls, the effect on these tissue structures due to cavitation achieved with selected frequencies and intensities of sonic energy, and the simple physical shaking of the system possible with various pulsed and modulated patterns of sonic energy, and the like.

When incorporating a sonic energy source into a system such as this, it is important to consider the acoustic impedance of the various layers through which the sound waves travel, and the matching of the acoustic impedance at the interfaces of the various layers. For diagnostic ultrasound, a gel is frequently used to facilitate the coupling of the sonic energy into the tissue and this approach could be used to mate the bottom surface of the integrated device element to the surface of the tissue, such as skin. An alternative solution to the coupling issue that eliminates the need for a coupling gel, is to use an appropriately designed gasket type of material, such as a silicone or hydrogel to form the sonic connection. In addition, tacky or adhesive elements are useful to both seal a fluid management chamber and maintain registration between the micropores and the inlet port of the assay system. These elements are also useful as efficient acoustic coupling agents.

In the case where a focused acoustic field is desired, multiple selectively phased sources, sonic lenses or reflectors could all be employed to generate the desired energy distribution within the target zone. A purposefully created impedance mismatch within the media through which the sound waves propagate can be used as a means of forming a reflective boundary. Basically, all traditional wave propagation equations hold true for sonic energy, just as they do for electromagnetic energy, and as such the same type of wave guide or energy directing methods can be employed to focus the sonic energy where desired.

The schematic representation in FIG. 19 shows an integrated device (100, 200, 300, 400, 600, 1000) having a compliant layer 900 placed on the top to form an efficient coupling for sonic energy. The sonic energy is generated by sonic energy generation means, such as a piezo-electric transducer 910. A sonic lens element 920 is placed between the piezo-electric transducer 910 and the compliant layer 900. A coupling gasket 930 may also be provided to pneumatically seal the integrated device to the surface of the tissue (with optional application of suction) and to assist in the acoustic coupling of the sonic energy.

The acoustic waves can be optimized to have any of several recognized actions and effects on the performance of the harvesting and analysis of biological fluid, or delivery of bio-active agents. The sonic energy can be propagated through the integrated device, through the coupling gasket 930, to the tissue (such as skin), wherein SC denotes the stratum corneum, E denotes the epidermis and D denotes the dermis.

Within the tissue, the direct effects of the sonic energy include local warming of the tissue through the direct absorption of the sonic energy. This is shown at reference numeral 940. Depending on the frequency selection and possible modulations of the frequency and amplitude of the sonic energy, an acoustic streaming effect can be achieved within the tissue, accelerating the fluidic movement between cells and within cells and vessels. This is shown at reference numeral 942. The amount of increase in the local velocity of the fluid has been shown to be more than one order of magnitude using visible tracers in in vivo real-time video microscopy experiments.

Similarly, when the frequency and intensity and possible modulation thereof are selected appropriately, a cavitation effect shown by cavitation bubbles at reference numeral 944, is achieved which can have substantial secondary effects on the tissue properties due to possible microscopic shearing of some tissue structures, the transitory opening up of micro-porous sites in various membranes such as the capillary walls CW within the tissue, and other effects due to the shock waves, shown at reference numeral 946, created upon the collapse of the cavitation bubble.

The presence of the acoustic vibrations within the fluid management chamber of the integrated device itself can also be used to enhance the motion of the fluid. These effects can be due to a directed radiation pressure gradient shown at reference numeral 948 which can be created by proper alignment and focusing of the sonic energy, the enhancement of capillary transport action shown at reference numeral 950 by the acoustic energy, the active out-gassing of dissolved gas in the fluid which can help to eliminate error causing bubbles in the active assay area of the system, and the localized and chaotic micro-fluidic vortices shown at reference numeral 950 created within the fluid management chamber which can be used to reduce the required assay reaction time by eliminating the dependency on passive diffusion effects and thereby evenly distribute the reactive process within the sample.

The activation of the sonic energy source can be selectively controlled to work in a coordinated fashion with the other components of the system, even to the point of operating with significantly different parameters during different portions of the poration, harvesting, assay process. For example, a sequence of sonic energy use is:

1. Start with a controlled burst of higher energy ultrasound designed to temporarily permeabilize the capillary walls and the intervening bulk tissue structures during the poration cycle. The presence of this type of short pulse of high intensity sonic energy has also been shown to reduce the perceived sensation associated with the thermal poration process by most subjects.

2. During the fluid collection phase, a lower power, swept frequency modulation setting of the sonic energy could be used to induce the acoustic streaming effect within the tissue designed to bring more biological fluid to the surface.

3. As the biological fluid exits the body and enters the inlet port of the assay system (the integrated device), the sonic energy could be re-tuned to more optimally enhance the surface tension driven transport of the biological fluid towards the active reagent area. Biological fluid transport could be used both within a capillary channel, a mesh or a porous media transport layer system.

4. Once on the active reagent layer, the operating parameters of the sonic energy could once again be adjusted to create the active “stirring” of the fluid within the fluid management chamber to facilitate a more rapid and/or accurate quantification of the selected analyte.

Essentially all of the same functional modalities described in conjunction with FIG. 19 can also be realized with an alternative configuration wherein a remotely placed sonic source is used to direct the acoustic energy towards the desired portion of the assay element of the integrated device by beaming it through a fold of intervening flesh.

With reference to FIG. 20, a clamp assembly 960 is provided to pinch a fold of tissue, such as skin between a transducer assembly. The transducer assembly comprises an acoustic transducer 962, a focusing element 964, and a coupling layer 966. The integrated device (100, 200, 300, 400 and 600) is at an opposite side of the pinch of skin. The dimensions of the clamp assembly 960 are such that when the tensioning device 968 pulls the two clamp halves together, they hit a hard stop and the spacing from the face of the transducer assembly and the inlet port of the fluid management chamber of the integrated device is positioned at an optimal position in {x, y, and z}coordinates to coincide with the sonic energy fields as desired. For example, FIG. 20 shows the focal point of the sonic field is roughly coincident with the inlet port of the assay chamber, which may be one selected mode of operation. However, by shifting the frequency of the sound waves, this focal point can be moved in and out from the face of the transducer.

Experiments have shown that it can be advantageous to modulate the frequency, thereby shifting the sonic energy field position and local intensities. This sort of control of sonic energy fields has been shown to induce an active pumping action at the modulation rate of the system which can similarly be used to exploit certain fluid and mechanical properties of the tissues.

By employing a clamping mechanism, which forces the sonic transducer against the skin surface, the coupling losses at this interface can be reduced and/or controlled within a design specification.

The initial deflection into the inter-clamp space can be accomplished by placing the entire assembly within a suction system, such as that shown in FIGS. 15 and 16, which pulls the flesh into the space, and as the vacuum increases, provides the clamping force to pull the two halves of the clamp assembly together to the stops. Similarly this could be accomplished via mechanically feeding a pinch of skin into the space and then letting the clamp grab the tissue.

An additional function of sonic energy applicable to all of the previously discussed sonic enhancement concepts is the demonstrated beneficial effects it can have on the wound healing process. Clinical results have consistently shown positive effects when sonic energy is applied to various types of wounds including burns and other superficial skin traumas. In the case of microporation created in the outer layers of the skin, this acceleration of the healing process can be exploited to improve the overall acceptance of the system by the end user and health care practitioners.

As discussed above, certain aspects of the present invention can be described in terms of methods including the formation of a micropore in the stratum corneum, removal and testing of a biological fluid, e.g., blood or interstitial fluid, therefrom as well as devices that integrate one or more of these steps.

These aspects of the invention can be accomplished by various state of the art means as well as certain means disclosed herein that are improvements thereof.

For example, the use of laser ablation as described by Jacques et al. in U.S. Pat. No. 4,775,361 and by Lane et al., supra, certainly provide one means for ablating the stratum corneum using an excimer laser. At 193 nm wavelength, and 14 ns pulsewidth, it was found that about 0.24 to 2.8 microns of stratum corneum could be removed by each laser pulse at radiant exposure of between about 70 and 480 mJ/cm². As the pulse energy increases, more tissue is removed from the stratum corneum and fewer pulses are required for complete poration of this layer. The lower threshold of radiant exposure that must be absorbed by the stratum corneum within the limit of the thermal relaxation time to cause suitable micro-explosions that result in tissue ablation is about 70 mJ/cm² within a 50 millisecond (ms) time. In other words, a total of 70 mJ/cm² must be delivered within a 50 ms window. This can be done in a single pulse of 70 mJ/cm² or in 10 pulses of 7 mJ/cm², or with a continuous illumination of 1.4 watts/cm² during the 50 ms time. The upper limit of radiant exposure is that which will ablate the stratum corneum without damage to underlying tissue and can be empirically determined from the light source, wavelength of light, and other variables that are within the experience and knowledge of one skilled in this art.

By “deliver” is meant that the stated amount of energy is absorbed by the tissue to be ablated. At the excimer laser wavelength of 193 nm, essentially 100% absorption occurs within the first 1 or 2 microns of stratum corneum tissue. Assuming the stratum corneum is about 20 μm thick, at longer wavelengths, such as 670 nm, only about 5% of incident light is absorbed within the 20 micron layer, This means that about 95% of the high power beam passes into the tissues underlying the stratum corneum where it will likely cause significant damage.

In this embodiment, the ideal is to use only as much power as is necessary to perforate the stratum corneum without causing bleeding, thermal, or other damage to underlying tissues from which analytes are to be extracted or drugs or other permeants delivered.

It would be beneficial to use sources of energy more economical than energy from excimer lasers. Excimer lasers, which emit light at wavelengths in the far UV region, are much more expensive to operate and maintain than, for example, diode lasers that emit light at wavelengths in visible and IR regions (600 to 1800 nm). However, at the longer wavelengths, the stratum corneum becomes increasingly more transparent and absorption occurs primarily in the underlying tissues.

This embodiment of the present invention facilitates a rapid and painless method of eliminating the barrier function of the stratum corneum to facilitate the transcutaneous transport of therapeutic substances into the body when applied topically or to access the analytes within the body for analysis.

This aspect of the invention includes embodiments that utilize a procedure that begins with the contact application of a small area heat source to the targeted area of the stratum corneum.

The heat source used in this embodiment should have several important properties, as will now be described.

First, the heat source should be sized such that contact with the skin is confined to a small area, typically about 1 to 1000 μm, in diameter. Second, it must have the capability to modulate the temperature of the stratum corneum at the contact point from ambient skin surface temperature levels (33° C.) to greater than 123° C. and then return to approximately ambient skin temperature with cycle times to minimize collateral damage to viable tissues and sensation to the subject individual. This modulation can be created electronically, mechanically, or chemically.

Additionally, an inherent depth limiting feature of the microporation process can be facilitated if the heat source has both a small enough thermal mass and limited energy source to elevate its temperature such that when it is placed in contact with tissues with more than 30% water content, the thermal dispersion in these tissues is sufficient to limit the maximum temperature of the heat source to less than 100° C. This feature effectively stops the thermal vaporization process once the heat probe had penetrated through the stratum corneum into the lower layers of the epidermis.

With the heat source placed in contact with the skin, it is cycled through a series of one or more modulations of temperature from an initial point of ambient skin temperature to a peak temperature in excess of 123° C. to approxmiately ambient skin temperature. To minimize or eliminate the subject's sensory perception of the microporation process, these pulses are limited in duration, and the interpulse spacing is long enough to allow cooling of the viable tissue layers in the skin, and most particularly the enervated dermal tissues, to acheive a mean temperature of less than about 45° C. These parameters are based on the thermal time constants of the viable epidermal tissues (roughly 30-80 msec) located between the heat probe and the enervated tissue in the underlying dermis. The result of this application of pulsed thermal energy is that enough energy is conducted into the stratum corneum within the tiny target spot that the local temperature of this volume of tissue is elevated sufficiently higher than the vaporization point of the tissue-bound water content in the stratum corneum. As the temperature increases above 100° C., the water content of the stratum corneum (typically 5% to 15%) within this localized spot, is induced to vaporize and expand very rapidly, causing a vapor-driven removal of those corneocytes in the stratum corneum located in proximity to this vaporization event.

U.S. Pat. No. 4,775,361 teaches that a stratum corneum temperature of 123° C. represents a threshold at which this type of flash vaporization occurs. As subsequent pulses of thermal energy are applied, additional layers of the stratum corneum are removed until a micropore is formed through the stratum corneum down to the next layer of the epidermis, the stratum lucidum. By limiting the duration of the heat pulse to less than one thermal time constant of the epidermis and allowing any heat energy conducted into the epidermis to dissipate for a sufficiently long enough time, the elevation in temperature of the viable layers of the epidermis is minimal. This allows the entire microporation process to take place without any sensation to the subject and no damage to the underlying and surrounding tissues.

This aspect of this present invention includes a method for painlessly creating microscopic holes, i.e. micropores, from about 1 to 1000 microns across, in the stratum corneum of human skin. The key to successfully implementing this method is the creation of an appropriate thermal energy source, or heat probe, which is held in contact with the stratum corneum. The principle technical challenge in fabricating all appropriate heat probe is designing a device that has the desired contact with the skin and that can be thermally modulated at a sufficiently high frequency.

It is possible to fabricate an appropriate heat probe by topically applying to the stratum corneum a suitable light-absorbing compound, such as a dye or stain, selected because of its ability to absorb light at the wavelength emitted by a selected light source. In this instance, the selected light source may be a laser diode emitting at a wavelength which would not normally be absorbed by the skin tissues. By focusing the light source to a small spot on the surface of the topical layer of the dye, the targeted area can be temperature modulated by varying the intensity of the light flux focused on it. It is possible to utilize the energy from laser sources emitting at a longer wavelength than an excimer laser by first topically applying to the stratum corneum a suitable light-absorbing compound, such as a dye or stain, selected because of its ability to absorb light at the wavelength emitted by the laser source.

The same concept can be applied at any wavelength and one must only choose an appropriate dye or stain and optical wavelength. One need only look to any reference manual to find which suitable dyes and wavelength of the maximum absorbance of that dye. One such reference is Green, The Sigma-Aldrich Handbook of Stains, Dyes and Indicators, Aldrich Chemical Company, Inc. Milwaukee, Wis. (1991).

For example, copper phthalocyanine (Pigment Blue 15; CPC) absorbs at about 800 nm; copper phthalocyanine tetrasulfonic acid (Acid Blue 249) absorbs at about 610 nm; and Indocyanine Green absorbs at about 775 nm; and Cryptocyanine absorbs at about 703 nm. CPC is particularly well suited for this embodiment for the following reasons: it is a very stable and inert compound, already approved by the FDA for use as a dye in implantable sutures; it absorbs very strongly at wavelengths from 750 nm to 950 nm, which coincide well with umerous low cost, solid state emitters such as laser diodes and LEDs, and in addition, this area of optical bandwidth is similarly not absorbed directly by the skin tissues in any significant amount; CPC has a very high vaporization point (>550° C. in a vacuum) and goes directly from a solid phase to a vapor phase with no liquid phase; CPC has a relatively low thermal diffusivity constant, allowing the light energy focused on it to selectively heat only that area directly in the focal point with very little lateral spreading of the ‘hot-spot’ into the surrounding CPC thereby assisting in the spatial definition of the contact heat-probe.

The purpose of this disclosure is not to make an exhaustive listing of suitable dyes or stains because such dyes and/or stains may be readily ascertained by one skilled in the art from data readily available.

The same is true for any desired particular pulsed light source. For example, this method may be implemented with a mechanically shuttered, focused incandescent lamp as the pulse light source. Various catalogs and sales literature show numerous lasers operating in the near UW, visible and near IR range. Representative lasers are Hammamatsu Photonic Systems Model PLP-02 which operates at a power output of 2 x10⁻⁸ J, at a wavelength of 415 nm; Hammamatsu Photonic Systems Model PLP-05 which operates at a power output of 15 J, at a wavelength of 685 nm; SDL, Inc., SDL-3250 Series pulsed laser which operates at a power output of 2×10⁶ J at a wavelength of about 800-810 nm; SDL, Inc., Model SDL-8630 which operates at a power output of 500 mW at a wavelength of about 670 nm; Uniphase Laser Model AR-081-15000 which operates at a power output of 15,000 mW at a wavelength of 790-830 nm; Toshiba America Electronic Model TOLD9150 which operates at a power output of 30 mW at a wavelength of 690 nm; and LiCONIX, Model Diolite 800-50 which operates at a power 50 mW at a wavelength of 780 nm.

For purposes of this aspect of the invention a pulsed laser light source can emit radiation over a wide range of wavelengths ranging from between about 100 nm to 12,000 nm. Excimer lasers typically will emit over a range of between about 100 to 400 nm. Commercial excimer lasers are currently available with wavelengths in the range of about 193 nm to 350 nm. Preferably a laser diode will have an emission range of between about 380 to 1550 nm. A frequency doubled laser diode will have an emission range of between about 190 and 775 nm. Longer wavelengths ranging from between about 1300 and 3000 nm may be utilized using a laser diode pumped optical parametric oscillator. It is expected, given the amount of research taking place on laser technology that these ranges will expand with time.

Delivered or absorbed energy need not be obtained from a laser as any source of light, whether it is from a laser, a short arc lamp such as a xenon flashlamp, an incandescent lamp, a light-emitting diode (LED), the sun, or any other source may be used. Thus, the particular instrument used for delivering electromagnetic radiation is less important than the wavelength and energy associated therewith. Any suitable instrument capable of delivering the necessary energy at suitable wavelengths, i.e., in the range of about 100 nm to about 12,000 nm, can be considered within the scope of this aspect of the invention. The essential feature of this emobidment is that the energy must be absorbed by the light-absorbing compound to cause localized heating thereof, followed by conduction of sufficient heat to the tissue to be ablated within the timeframe allowed.

In one illustrative embodiment, the heat probe itself is formed from a thin layer, preferably about 5 to 1000 microns thick, of a solid, non-biologically active compound, applied topically to a selected area of an individual's skin that is large enough to cover the site where a micropore is to be created. The specific formulation of the chemical compound is chosen such that it exhibits high absorption over the spectral range of a light source selected for providing energy to the light-absorbing compound. The probe can be, for example, a sheet of a solid compound, a film treated with a high melting point absorbing compound, or a direct application of the light-absorbing compound to the skin as a precipitate or as a suspension in a carrier. Regardless of the configuration of the light-absorbing heat probe, it must exhibit a low enough lateral thermal diffusion coefficient such that any local elevations of temperature will remain spatially defined and the dominant mode of heat loss will be via direct conduction into the stratum corneum through the point of contact between the skin and the probe.

The required temperature modulation of the probe can be achieved by focusing a light source onto the light-absorbing compound and modulating the intensity of this light source. If the energy absorbed within the illuminated area is sufficiently high, it will cause the light absorbing compound to rapidly heat up. The amount of energy delivered, and subsequently both the rate of heating and peak temperature of the light-absorbing compound at the focal point, can be easily modulated by varying the pulse width and peak power of the light source. In this embodiment, it is only the small volume of light-absorbing compound heated up by the focused, incident optical energy that forms the heat probe, additional light absorbing compound which may have been applied over a larger area then the actual poration site is incidental. By using a solid phase light-absorbing compound with a relatively high melting point, such as copper phthalocyanine (CPC), which remains in its solid phase up to a temperature of greater than 550° C., the heat probe can be quickly brought up to a temperature of several hundred degrees C., and still remain in contact with the skin, allowing this thermal energy to be conducted into the stratum corneum. In addition, this embodiment comprises choosing a light source with an emission spectrum where very little energy would normally be absorbed in the skin tissues.

Once the targeted area has the light-absorbing compound topically positioned on it, the heat probe is formed when the light source is activated with the focal waist of the beam positioned to be coincident with the surface of the treated area. The energy density of light at the focal waist and the amount of absorption taking place within the light-absorbing compound are set to be sufficient to bring the temperature of the light-absorbing compound, within the area of the small spot defined by the focus of the light source, to greater than 123° C. within a few milliseconds. As the temperature of the heat probe rises, conduction into the stratum corneum delivers energy into these tissues, elevating the local temperature of the stratum corneum. When enough energy has been delivered into this small area of stratum corneum to cause the local temperature to be elevated above the boiling point of the water contained in these tissues, a flash vaporization of this water takes place, ablating the stratum corneum at this point.

By turning the light source on and off, the temperature of the heat probe can be rapidly modulated and the selective ablation of these tissues can be achieved, allowing a very precisely dimensioned hole to be created, which selectively penetrates only through the first 10 to 30 microns of skin.

An additional feature of this embodiment is that by choosing a light source that would normally have very little energy absorbed by the skin or underlying tissues, and by designing the focusing and delivery optics to have a sufficiently high numerical aperture, the small amount of delivered light that does not happen to get absorbed in the heat probe itself, quickly diverges as it penetrates deep into the body. Since there is very little absorption at the delivered wavelengths, essentially no energy is delivered to the skin directly from the light source. This three dimensional dilution of coupled energy in the tissues due to beam divergence and the low level of absorption in the untreated tissue results in a completely benign interaction between the light beam and the tissues, with no damage being done thereby.

In one example of this embodiment, a laser diode is used as the light source with an emission wavelength of 900±30 nm. A heat-probe can be formed by topical application of a transparent adhesive tape that has been treated on the adhesive side with a 0.5 cm² spot formed from a deposit of finely ground copper phthalocyanine (CPC). The CPC exhibits extremely high absorption coefficients in the 800 nm spectral range, typically absorbing more than 95% of the radiant energy from a laser diode.

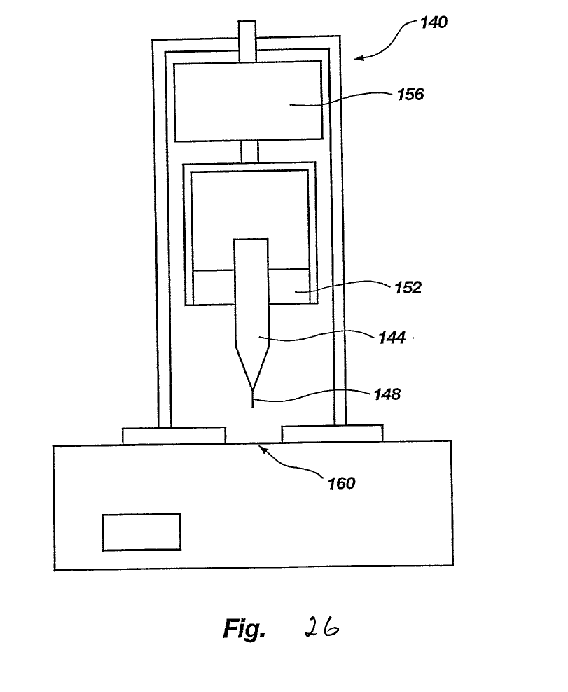

FIG. 23 shows a system 10 for delivering light from such a laser diode to a selected area of an individual's skin and for monitoring the progress of the poration process. The system comprises a laser diode 14 coupled to a controller 18, which controls the intensity, duration, and spacing of the light pulses. The laser diode emits a beam 22 that is directed to a collection lens or lenses 26, which focuses the beam onto a mirror 30. The beam is then reflected by the mirror to an objective lens or lenses 34, which focuses the beam at a preselected point 38. This preselected point corresponds with the plane of an xyz stage 42 and the objective hole 46 thereof, such that a selected area of an individual's skin can be irradiated. The xyz stage is connected to the controller such that the position of the xyz stage can be controlled. The system also comprises a monitoring system comprising a CCD camera 50 coupled to a monitor 54 The CCD camera is confocally aligned with the objective lens such that the progress of the poration process can be monitored visually on the monitor.

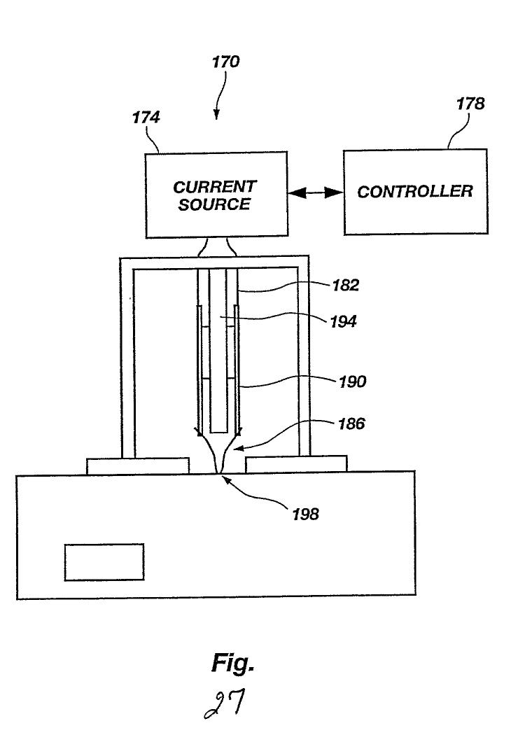

In another illustrative embodiment of the invention, a system of sensing photodiodes and collection optics that have been confocally aligned with the ablation light source is provided. FIG. 24 shows a sensor system 60 for use in this embodiment. The system comprises a light source 64 for emitting a beam of light 68, which is directed through a delivery optics system 72 that focuses the beam at a preselected point 76, such as the surface of an individual's skin 80. A portion of the light contacting the skin is reflected, and other light is emitted from the irradiated area. A portion of this reflected and emitted light passes through a filter 84 and then through a collection optics system 88, which focuses the light on a phototodiode 92. A controller 96 is coupled to both the laser diode and the photodiode for, respectively, controlling the output of the laser diode and detecting the light that reaches the photodiode. Only selected portions of the spectrum emitted from the skin pass through the filter. By analyzing the shifts in the reflected and emitted light from the targeted area, the system has the ability to detect when the stratum corneum has been breached, and this feedback is then used to control the light source, deactivating the pulses of light when the microporation of the stratum corneum is achieved. By employing this type of active closed loop feedback system, a self regulating, universally applicable device is obtained that produces uniformly dimensioned micropores in the stratum corneum, with minimal power requirements, regardless of variations from one individual to the next.

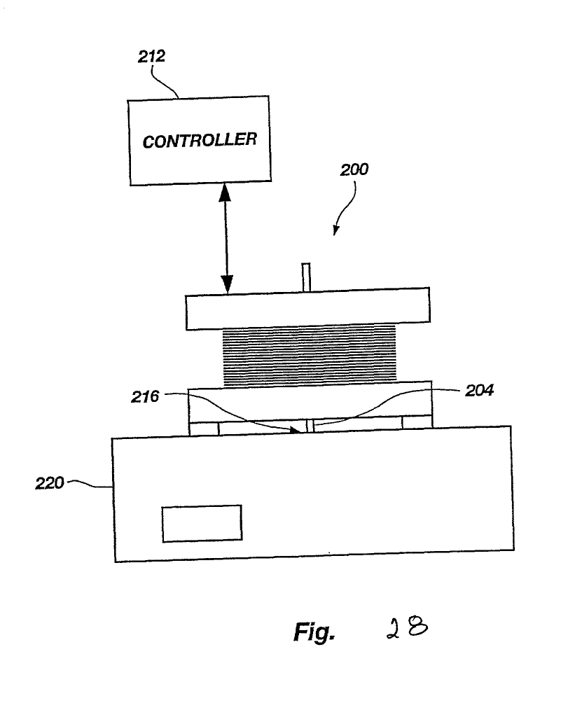

In another illustrative embodiment, a cooling device is incorporated into the system interface to the skin. FIG. 25A shows an illustrative schematic representation thereof. In this system 100, a light source 104 (coupled to a controller 106) emits a beam of light 108, which passes through and is focused by a delivery optics system 112. The beam is focused by the delivery optics system to a preselected point 116, such as a selected area of an individual's skin 120. A cooling device 124, such as a Peltier device or other means of chilling, contacts the skin to cool the surface thereof. In a preferred embodiment of the cooling device 124 (FIG. 25B), there is a central hole 128 through which the beam of focused light passes to contact the skin. Referring again to FIG. 25A, a heat sink 132 is also preferably placed in contact with the cooling device. By providing a cooling device with a small hole in its center coincident with the focus of the light, the skin tissues in the general area where the poration is to be created may be pre-cooled to 5° C. to 10° C. This pre-cooling allows a greater safety margin for the system to operate in that the potential sensations to the user and the possibility of any collateral damage to the epidermis directly below the poration site are reduced significantly from non-cooled embodiment. Moreover, for monitoring applications, pre-cooling minimizes evaporation of interstitial fluid and can also provide advantageous physical properties, such as decreased surface tension of such interstitial fluid. Still further, cooling the tissue is known to cause a localized increase in blood flow in such cooled tissue, thus promoting diffusion of analytes from the blood into the interstitial fluid.

The method can also be applied for other micro-surgery techniques wherein the light-absorbing compound/heat-probe is applied to the area to be ablated and then the light source is used to selectively modulate the temperature of the probe at the selected target site, affecting the tissues via the vaporization-ablation process produced.

A further feature of this embodiment can include the use of a light source to help seal the micropore after its usefulness has passed. Specifically, in the case of monitoring for an internal analyte, a micropore is created and some amount of interstitial fluid is extracted through this opening. After a sufficient amount of interstitial fluid had been collected, the light source is reactivated at a reduced power level to facilitate rapid clotting or coagulation of the interstitial fluid within the micropore. By forcing the coagulation or clotting of the fluid in the pore, this opening in the body can be effectively sealed, thus reducing the risk of infection. Also, the use of the light source itself for both the formation of the micropore and the sealing thereof is an inherently sterile procedure, with no physical penetration into the body by any device or apparatus.

Further, the thermal shock induced by the light energy kills any microbes that may happen to be present at the ablation site.

This concept of optical sterilization can be extended to include an additional step in the process wherein the light source is first applied in an unfocused manner, covering the target area with an illuminated area that extends 100 μm or more beyond the actual size of the micropore to be produced. By selecting the area over which the unfocused beam is to be applied, the flux density can be correspondingly reduced to a level well below the ablation threshold but high enough to effectively sterilize the surface of the skin. After a sufficiently long exposure of the larger area, either in one continuous step or in a series of pulses, to the sterilizing beam, the system is then configured into the sharply focused ablation mode and the optical microporation process begins.

Another illustrative embodiment of the invention is to create the required heat probe from a metallic solid, such as a small diameter wire. As in the previously described embodiment, the contacting surface of the heat probe must be able to have its temperature modulated from ambient skin temperatures (33° C.) to temperatures greater than 123° C., within the required time allowed of, preferably, between about 1 to 50 msec at the high temperature (on-time) and at least about 10 to 50 msec at the low temperature (off-time). In particular, being able to modulate the temperature up to greater than 150° C. for an “on” time of around 5 msec and an off time of 50 msec produces very effective thermal ablation with little or no sensation to the individual.

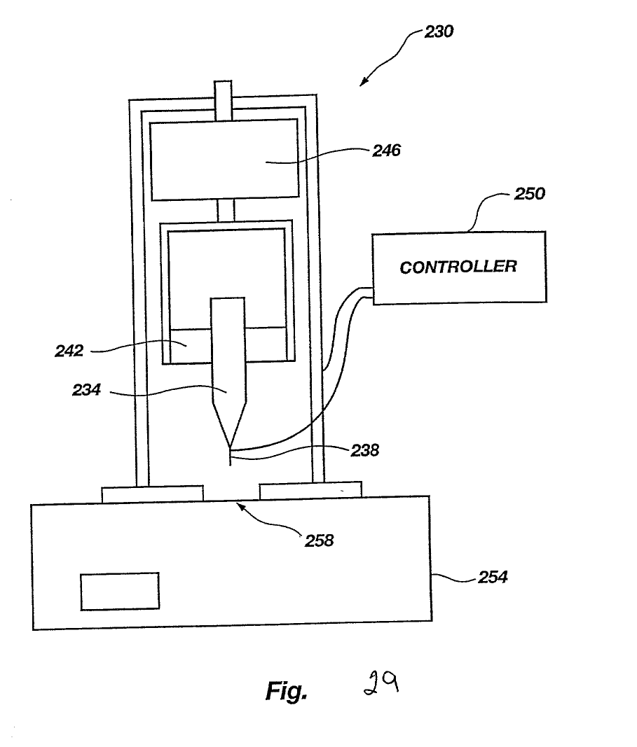

Several methods for modulating the temperatures of the wire heat probe contact area may be successfully implemented. For example, a short length of wire may be brought up to the desired high temperature by an external heating element such as an ohmic heating element used in the tip of a soldering iron. FIG. 26 shows an ohmic heating device 140 with a mechanical actuator. The ohmic heating device comprises an ohmic heat source 144 coupled to a wire heat probe 148. The ohmic heat source is also coupled through an insulating mount 152 to a mechanical modulation device 156, such as a solenoid. In this configuration, a steady state condition can be reached wherein the tip of the wire probe will stabilize at some equilibrium temperature defined by the physical parameters of the structure, i.e., the temperature of the ohmic heat source, the length and diameter of the wire, the temperature of the air surrounding the wire, and the material of which the wire is comprised. Once the desired temperature is achieved, the modulation of the temperature of the selected area of an individual's skin 160 is effected directly via the mechanical modulation device to alternatively place the hot tip of the wire in contact with the skin for, preferably, a 5 msec on-time and then withdraw it into the air for, preferably, a 50 ms off-time.

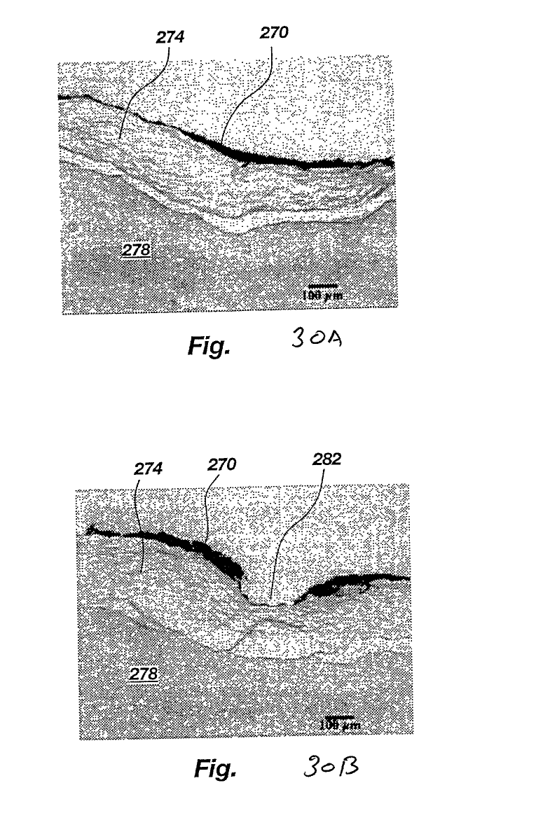

Another illustrative example (FIG. 27), shows a device 170 comprising a current source 174 coupled to a controller 178. The current source is coupled to a current loop 182 comprising a wire 186 formed into a structure such that it presents a high resistance point. Preferably, the wire is held on a mount 190, and an insulator 194 separates different parts of the current loop. The desired modulation of temperature is then achieved by merely modulating the current through the wire. If the thermal mass of the wire element is appropriately sized and the heat sinking provided by the electrodes connecting it to the current source is sufficient, the warm-up and cool-down times of the wire element can be achieved in a few milliseconds. Contacting the wire with a selected area of skin 198 heats the stratum corneum to achieve the selected ablation.

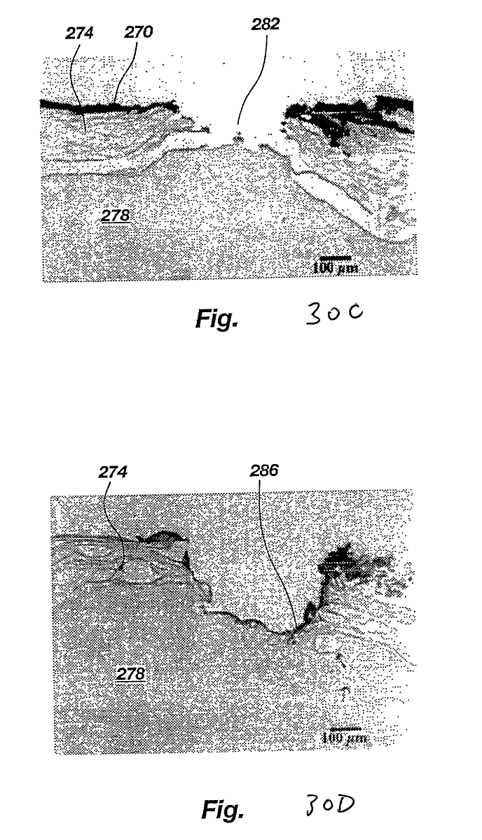

In FIG. 28 there is shown still another illustrative example of porating the stratum corneum with a hot wire. In this system 200, the wire 204 can be positioned within a modulatable alternating magnetic field formed by a coil of wire 208, the excitation coil. By energizing the alternating current in the excitation coil by means of a controller 212 coupled thereto, eddy currents can be induced in the wire heat probe of sufficient intensity that it will be heated up directly via the internal ohmic losses. This is essentially a miniature version of an inductive heating system commonly used for heat treating the tips of tools or inducing outgassing from the electrodes in vacuum or flash tubes. The advantage of the inductive heating method is that the energy delivered into the wire heat probe can be closely controlled and modulated easily via the electronic control of the excitation coil. If the thermal mass of the wire probe itself, and the thermal mass of the stratum corneum in contact with the tip of the probe are known, controlling the inductive energy delivered can produce very precise control of the temperature at the contact point 216 with the skin 220. Because the skin tissue is essentially non-magnetic at the lower frequencies at which inductive heating can be achieved, if appropriately selected frequencies are used in the excitation coil, then this alternating electromagnetic field will have no effect on the skin tissues.

If a mechanically controlled contact modulation is employed, an additional feature may be realized by incorporating a simple closed loop control system wherein the electrical impedance between the probe tip and the subject's skin is monitored. In this manner, the position of the probe can be brought into contact with the subject's skin, indicated by the stepwise reduction in resistance once contact is made, and then held there for the desired “on-time,” after which it can be withdrawn. Several types of linear actuators are suitable for this forin of closed loop control, such as a voice-coil mechanism, a simple solenoid, a rotary system with a cam or bell-crank, and the like. The advantage is that as the thermal ablation progresses, the position of the thermal probe tip can be similarly advanced into the skin, always ensuring good a contact to facilitate the efficient transfer of the required thermal energy. Also, the change in the conductivity properties of the stratum corneum and the epide rmis can be used to provide an elegant closed loop verification that the poration process is complete, i.e., when the resistance indicates that the epidermis has been reached, it is time to stop the poration process.

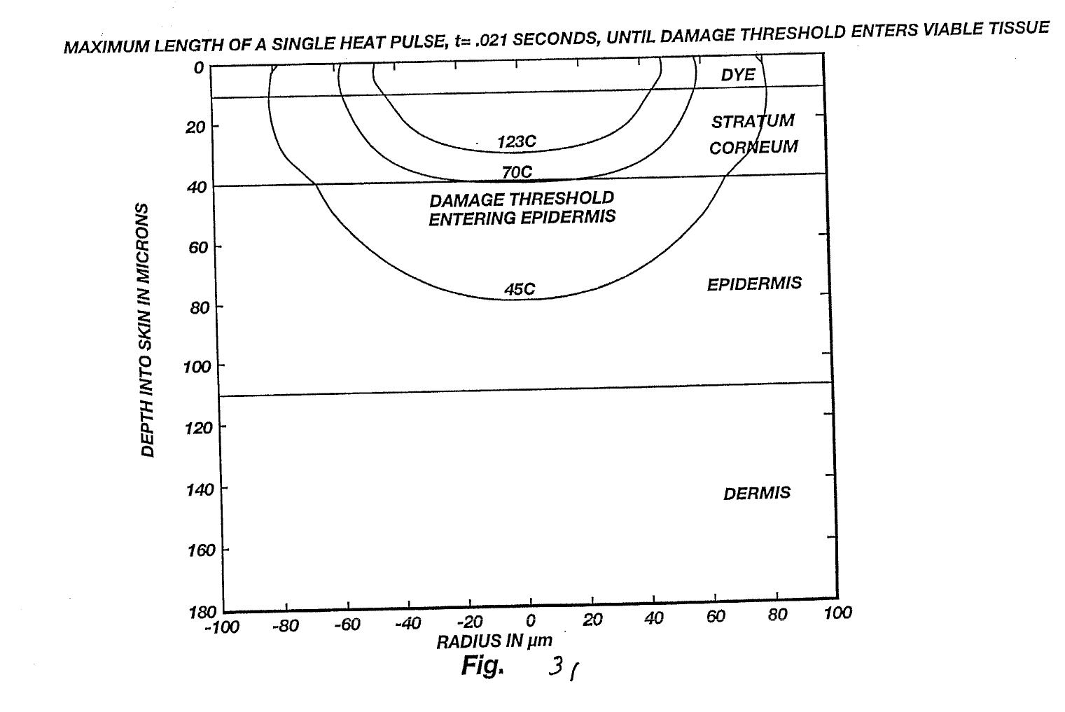

FIG. 29 shows an illustrative example of such a closed loop impedance monitor. In this system 230, there is an ohmic heat source 234 coupled to a wire heat probe 238. The heat source is mounted through an insulating mount 242 on a mechanical modulator 246. A controller 250 is coupled to the wire and to the skin 254, wherein the controller detects changes in impedance in the selected area 258 of skin, and when a predetermined level is obtained the controller stops the poration process.

In this aspect of the invention, along the same line as hydraulic poration means are microlancets adapted to just penetrate the stratum corneum for purposes of administering a permeant, such as a drug, through the pore formed or to withdraw an analyte through the pore for analysis. Such a device is considered to be “minimally invasive” as compared to devices and/or techniques that are non-invasive.

The use of micro-lancets that penetrate below the stratum corneum for withdrawing blood are well known. Such devices are cotrimercially available from manufacturers such as Becton-Dickinson and Lifescan and can be utilized in the present invention by controlling the depth of penetration. As an example of a micro-lancet device for collecting body fluids, reference is made to Erickson et al., International Published PCT Application WO 95/10223 (published Apr. 20, 1995). This application shows a device for penetration into the dermal layer of the skin, without penetration into subcutaneous tissues, to collect body fluids for monitoring, such as for blood glucose levels.

Poration of stratum corneum can also be accomplished using sonic means.

Sonic-poration is a variation of the optical means described above except that, instead of using a light source, a very tightly focused beam of sonic energy is delivered to the area of the stratum corneum to be ablated. The same levels of energy are required, i.e. a threshold of 70 mJ/cm²/50 msec still must be absorbed. The same pulsed focused ultrasonic transducers as described in parent applications Ser. Nos. 08/152,442 and 08/152,174 can be utilized to deliver the required energy densities for ablation as are used in the delivery of sonic energy which is modulated in intensity, phase, or frequency or a combination of these parameters for the transdermal sampling of an analyte or the transdermal delivery of drugs. This has the advantage of allowing use of the same transducer to push a drug through the stratum corneum or pull a body fluid to the surface for analysis to be used to create a micropore.

Additionally, electroporation or short bursts or pulses of electrical current can be delivered to the stratum corneum with sufficient energy to form micropores. Electroporation is known in the art for producing pores in biological membranes and electroporation instruments are commercially available. Thus, a person of skill in this art can select an instrument and conditions for use thereof without undue experimentation according to the guidelines provided herein.

The micropores produced in the stratum corneum by the methods of the present invention allow high flux rates of large molecular weight therapeutic compounds to be delivered transdermally. In addition, these non-traumatic microscopic openings into the body allow access to various analytes within the body, which can be assayed to determine their internal concentrations.

### EXAMPLE 1

In this example, skin samples were prepared as follows. Epidermal membrane was separated from human cadaver whole skin by the heat-separation method of Klingman and Christopher, 88 Arch. Dermatol. 702 (1963), involving the exposure of the full thickness skin to a temperature of 60° C. for 60 seconds, after which time the stratum corneum and part of the epidermis (epidermal membrane) were gently peeled from the dermis.

### EXAMPLE 2

Heat separated stratum corneum samples prepared according to the procedure of Example 1 were cut into cm² sections. These small samples were than attached to a glass cover slide by placing them on the slide and applying an pressure sensitive adhesive backed disk with a 6 mm hole in the center over the skin sample. The samples were then ready for experimental testing. In some instances the skin samples were hydrated by allowing them to soak for several hours in a neutral buffered phosphate solution or pure water.

As a test of these untreated skin samples, the outputs of several different infrared laser diodes, emitting at roughly 810, 905, 1480 and 1550 nanometers were applied to the sample. The delivery optics were designed to produce a focal waist 25 microns across with a final objective have a numerical aperture of 0.4. The total power delivered to the focal point was measured to be between 50 and 200 milliwatts for the 310 and 1480 nm laser diodes, which were capable of operating in a continuous wave (CW) fashion. The 905 and 1550 nm laser diodes were designed to produce high peak power pulses roughly 10 to 200 nanoseconds long at repetition rates up to 5000 Hz. For the pulsed lasers the peak power levels were measured to be 45 watts at 905 nm. and 3.5 watts at 1550 nm.

Under these operating conditions, there was no apparent effect on the skin samples from any of the lasers. The targeted area was illuminated continuously for 60 seconds and then examined microscopically, revealing no visible effects. In addition, the sample was placed in a modified Franz cell, typically used to test transdermal delivery systems based on chemical permeation enhancers, and the conductivity from one side of the membrane to the other was measured both before and after the irradiation by the laser and showed no change. Based on these tests that were run on skin samples from four different donors, it was concluded that at these wavelengths the coupling of the optical energy into the skin tissue was so small that no effects are detectable.

### EXAMPLE 3

To evaluate the potential sensation to a living subject when illuminated with optical energy under the conditions of Example 2, six volunteers were used and the output of each laser source was applied to their fingertips, forearms, and the backs of their hands. In the cases of the 810, 905 and 1550 nm lasers, the subject was unable to sense when the laser was turned on or off. In the case of the 1480 nm laser, there was a some sensation during the illumination by the 1480 nm laser operating at 70 mW CW, and a short while later a tiny blister was formed tinder the skin due to the absorption of the 1480 nm radiation by one of the water absorption bands. Apparently the amount of energy absorbed was sufficient to induce the formation of the blister, but was not enough to cause the ablative removal of the stratum corneum. Also, the absorption of the 1480 nm light occurred predominantly in the deeper, fully hydrated (85% to 90% water content) tissues of the epidermis and dermis, not the relatively dry (10% to 15% water content) tissue of the stratum corneum.

### EXAMPLE 4

Having demonstrated the lack of effect on the skin in its natural state (Example 3), a series of chemical compounds was evaluated for effectiveness in absorbing the light energy and then transferring this absorbed energy, via conduction, into the targeted tissue of the stratum corneum. Compounds tested included India ink; “SHARPIE” brand indelible black, blue, and red marking pens; methylene blue; fuschian red; epolite #67, an absorbing compound developed for molding into polycarbonate lenses for protected laser goggles; tincture of iodine; iodine-polyvinylpyrrolidone complex (“BETADINE”); copper phthalocyanine; and printers ink.

Using both of the CW laser diodes described in Example 2, positive ablation results were observed on the in vitro samples of heat-separated stratum corneum prepared according to Example 1 when using all of these products, however some performed better than others. In particular the copper phthalocyanine (CPQ) and the epolite 467 were some of the most effective. One probable reason for the superior performance of the CPC is its high boiling point of greater the 500° C. and the fact that it maintains its solid phase up to this temperature.

### EXAMPLE 5

As copper phthalocyanine has already been approved by the FDA for use in implantable sutures, and is listed in the Merck index as a rather benign and stable molecule in regard to human biocompatability, the next step taken was to combine the topical application of the CPC and the focused light source to the skin of healthy human volunteers. A suspension of finely ground CPC in isopropyl alcohol was prepared. The method of application used was to shake the solution and then apply a small drop at the target site. As the alcohol evaporated, a fine and uniform coating of the solid phase CPC was then left on the surface of the skin.

The apparatus shown in FIG. 23 was then applied to the site, wherein the CPC had been topically coated onto the skin, by placing the selected area of the individual's skin against a reference plate. The reference plate consists of a thin glass window roughly 3 cm×3 cm, with a 4 mm hole in the center. The CPC covered area was then positioned such that it was within the central hole. A confocal video microscope (FIG. 23) was then used to bring the surface of the skin into sharp focus. Positioning the skin to achieve the sharpest focus on the video system also positioned it such that the focal point of the laser system was coincident with the surface of the skin. The operator then activated the pulses of laser light while watching the effects at the target site on the video monitor. The amount of penetration was estimated visually by the operator by gauging the amount of defocusing of the laser spot in the micropore as the depth of the micropore increased, and this can be dynamically corrected by the operator, essentially following the ablated surface down into the tissues by moving the position of the camera/laser source along the ‘Y’ axis, into the skin. At the point when the stratum corneum had been removed down to the epidermis, the appearance of the base of the hole changed noticeably, becoming much wetter and shinier. Upon seeing this change, the operator deactivated the laser. In many instances, depending on the state of hydration of the subject as well as other physiological conditions, a dramatic outflow of interstitial fluid occurred in response to the barrier function of the stratum corneum being removed over this small area. The video system was used to record this visual record of the accessibility of interstitial fluid at the poration site.

### EXAMPLE 6

The procedure of Example 5 was followed except that the CPC was applied to a transparent adhesive tape, which was then caused to adhere to a selected site on the skin of an individual. The results were substantially similar to those of Example 5.

### EXAMPLE 7

Histology experiments were performed on cadaver skin according to methods well known in the art to determine ablation threshold parameters for given dye mixtures and collateral damage information. The top surface of the skin sample was treated with a solution of copper phthalocyanine (CPC) in alcohol. After the alcohol evaporated, a topical layer of solid phase CPC was distributed over the skin surface with a mean thickness of 10 to 20 um. FIG. 30A shows a cross-section of full thickness skin prior to the laser application, wherein the CPC layer 270, stratum comeurn 274, and underlying epidermal layers 278 are shown. FIG. 30B shows the sample after a single pulse of 810 nm light was applied to an 80 μm diameter circle with an energy density of 4000 J/cm², for a pulse period of 20 msec. It is noteworthy that there was still a significant amount of CPC present on the surface of the stratum corneum even in the middle of the ablated crater 282. It should also be noted that laboratory measurements indicate that only about 10% of the light energy incident on the CPC is actually absorbed, with the other 90% being reflected or backscattered. Thus the effective energy flux being deliverd to the dye layer which could cause the desired heating is only about 400 J/cm². 8C shows the sample after 5 pulses of 810 nm light were applied, wherein the stratum corneum barrier was removed with no damage to the underlying tissue. These results are a good representation of the “ideal” optically modulated thermal ablation performance. FIG. 30D shows the sample after 50 pulses were applied. Damaged tissue 286 was present in the epidermal layers due to carbonization of non ablated tissue and thermal denaturing of the underlying tissue. FIGS. 30A-30C show separations between the stratum corneum and the underlying epidermal layers due to an artifact of dehydration, freezing, and preparations for imaging.

### EXAMPLE 8

To examine the details of the thermal ablation mechanism, a mathematical model of the skin tissues was constructed upon which various different embodiments of the thermal ablation method could be tried. This model computes the temperature distribution in a layered semi-infinite medium with a specified heat flux input locally on the surface and heat removal from the surface some distance away, i.e. convection is applied between the two. The axisymmetric, time-dependent diffusion equation is solved in cylindrical coordinates using the alternating-direction-implicit (ADI) method. (Note: Constant Temp. B.C. is applied on lower boundary to serve as z→inf, and zero radial heat flux is applied on max radial boundary to serve as r→inf). The layers are parallel to the surface and are defined as: (1) dye; (2) stratum corneum; (3) underlying epidermis; and (4) dermis. The depth into the semi-infinite medium and thermal properties, density (rho), specific heat (c), and conductivity (k) must be specified for each layer.

First, a heat-transfer coefficient, h, on the skin is computed based on the “steady,” “1D,” temperature distribution determined by the ambient air temperature, skin surface temperature, and dermis temperature. It is assumed that there is no dye present and provides “h” on the skin surface. The program then allows one to use this “h” on the dye layer surface or input another desired “h” for the dye surface. Next, the “steady” temperature distribution is computed throughout all layers (including the dye layer) using the specified “h” at the dye surface. This temperature distribution is the initial condition for the time-dependent heating problem. This constitutes the “m-file” initial.m. The program then solves for the timedependent temperature distribution by marching in time, computing and displaying the temperature field at each step.

Each embodiment of the method described herein, for which empirical data have been collected, has been modeled for at least one set of operational parameters, showing how stratum corneum ablation can be achieved in a precise and controllable fashion. The output of the simulations is presented graphically in two different formats: (1) a cross-sectional view of the skin showing the different tissue layers with three isotherms plotted on top of this view which define three critical temperature thresholds, and (2) two different temperature -vs- time plots, one for the point in the middle of the stratum corneum directly beneath the target site, and the second for the point at the boundary of the viable cell layers of the epidennis and the underside of the stratum corneum. These plots show how the temperature at each point varies with time as the heat pulses are applied as if one could implant a microscopic thermocouple into the tissues. In addition, the application of this model allows investigation of the parametric limits within which the method can be employed to set the outer limits for two important aspects of the methods perfon-nance. First, general cases are presented cases that define the envelope within which the method can be employed without causing pain or undesired tissue damage.

For any given heat source, as described in the several different embodiments of the invention, there is a point at which the effect on the subject's skin tissues becomes non-optimal in that the subject perceives a pain sensation, or that the viable cells in the underlying epidermis and/or dermis sustain temperatures, which if maintained for a long enough duration, will render damage to these tissues. Accordingly, a test simulation was run using the optically heated topical copper plithalocyanine (CPC) dye embodiment as a baseline method to establish how the thermal time constants of the different skin tissue layers essentially define a window within which the method can be employed without pain or damage to adjacent tissue layers.

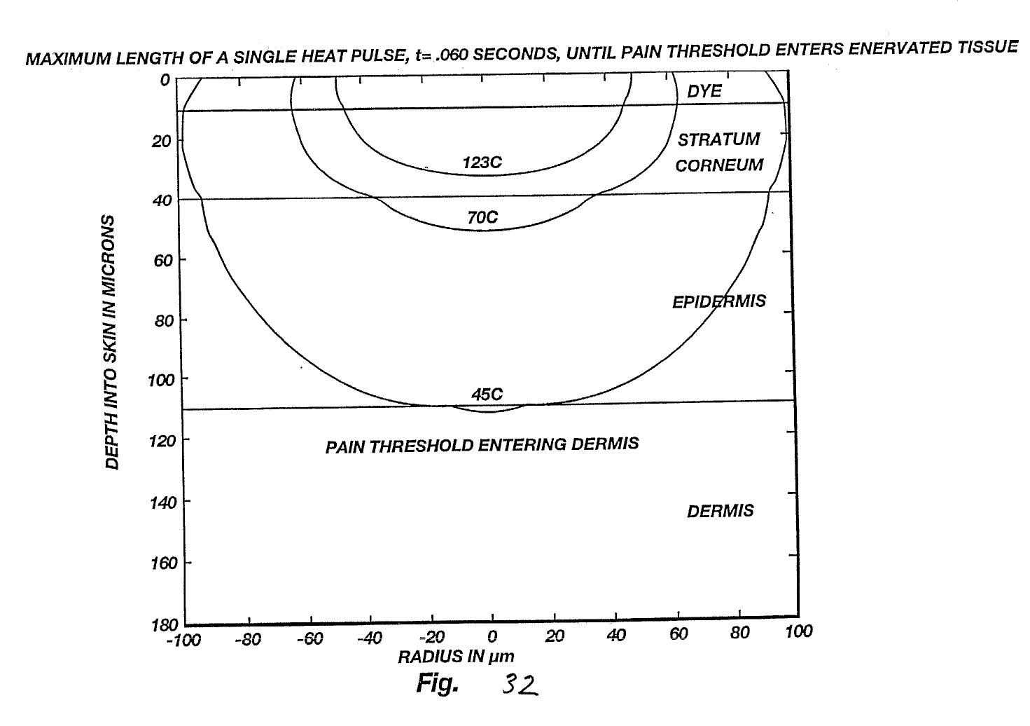

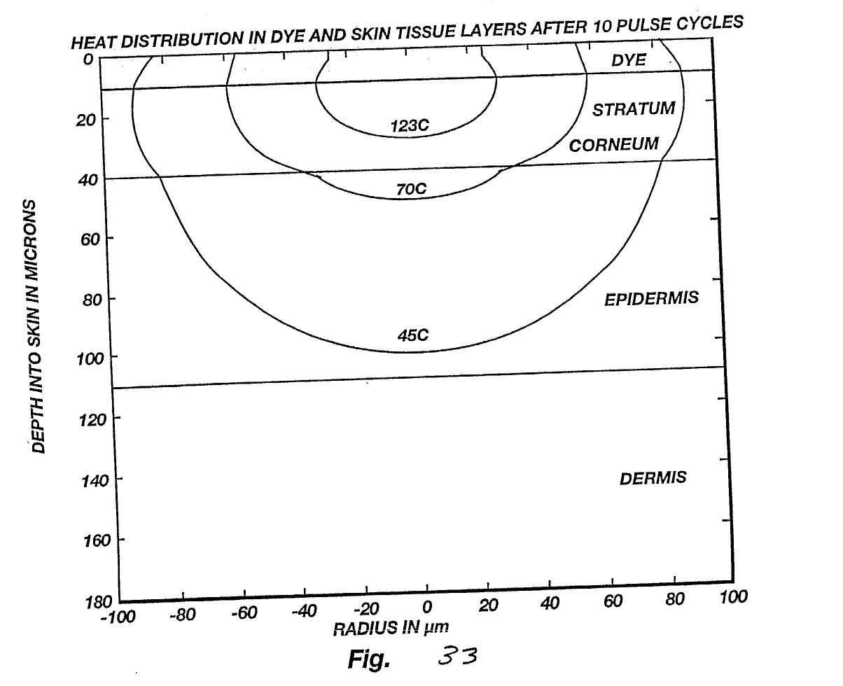

FIGS. 31 and 32 show schematic cross-sectional views of the skin and the topical dye layer. In each figure, three distinct isotherms are displayed: (1) 123° C., the point at which vaporization of the water in the tissue produces an ablation of the tissue; (2) 70 C., the point at which viable cells will be damaged if this temperature is maintained for several seconds; and (3) 45° C., the average point at which a sensation of pain will be perceived by the subject. This pain threshold is described in several basic physiology texts, but experience shows this threshold to be somewhat subjective. In fact, in repeated tests on the same individual, different poration sites within a few millimeters of each other can show significantly different amounts of sensation, possibly due to the proximity to a nerve ending in relationship to the poration site.

The dimensions on the graphs show the different layers of the dye and skin, as measured in microns, with flat boundaries defining them. Whereas the actual skin tissues have much more convoluted boundaries, in a mean sense for the dimensions involved, the model provides a good approximation of the thermal gradients present in the actual tissues. The dimensions used in this, and all subsequent simulations, for the thicknesses of the CPC dye layer and the various skin layers are as follows: dye, 10 microns; stratum corneum, 30 microns; underlying epidermis, 70 microns; and dermis, 100 microns.

Additional conditions imposed on the model for this particular simulation are shown in the following tables:

TABLE I
Initial Conditions for Finite Difference Thermal Model

| | |
|---|---|
| Ambient Air Temperature | Ta = 20° C. |
| Skin Surface Temperature | Ts = 30° C. |
| Dermis Temperature | Td = 37° C. |
| Dye Vaporization Temperature | Tvap = 550° C. |
| S.C. Vaporization Temperature | Tc1 = 123° C. |
| Tissue Damage Temperature | Tc2 = 70° C. |
| “Pain” Temperature | Tc3 = 45° C. |
| Radius of Irradiated Area | Rₕₒₜ = 30 μm |
| Energy Density Applied | FLUX = 400 |
| Joules/cm² | |

TABLE 2

| Parameter | Dye | S.C. | Epidermis | Dermis |
|---|---|---|---|---|
| Thermal Conductivity | 0.00046 | .00123 | 0.00421 | 0.00421 |
| Density | 0.67 | 1.28 | 1.09 | 1.09 |
| Specific Heat | 0.8 | 1.88 | 3.35 | 3.35 |

When these simulations are run, the following conservative assumptions are imposed:

1. While some portion of the stratum corneum may be shown as having a temperature already exceeded the ablation threshold for thermal vaporization of the water content, this event is not modeled, and the subsequent loss of heat energy in the tissues due to this vaporization is not factored into the simulation. This will cause a slight elevation in the temperatures shown in the underlying tissues from that point on in the simulation run.

2. Similarly, when some portion of the copper phthalocyanine (CPQ dye layer is shown to have reached its vaporization point of 550° C., this event is not modeled, but the temperature is merely hard-limited to this level. This will also cause a slight elevation of the subsequent temperatures in the underlying layers as the simulation progresses.

Even with these simplifications used in the model, the correlation between the predicted performance and the empirically observed perforinance based on both clinical studies and histological studies on donor tissue samples is remarkable. The key data to note in FIGS. 31 and 32 are the length of time that the heat pulse is applied, and the location of the three different threshold temperatures displayed by the isotherms.

In FIG. 31, with a pulse length of 21 milliseconds, the 70° C. isotherm just crosses the boundary separating the stratum corneum and the viable cell layers in the epidermis. In in vitro studies on donor skin samples under these conditions, fifty pulses of thermal energy delivered 50 milliseconds apart cause detectable damage to this top layer of living cells (see FIG. 30)). However, it was also shown in the in vitro studies that five pulses of heat energy at these same operating parameters, did not produce any significant damage to these tissues. It seems reasonable that even though the nominal damage threshold may have been exceeded, at feast in a transient sense, this temperature must be maintained for some cumulative period of time to actually cause any damage to the cells. Nevertheless, the basic information presented by the simulation is that if one keeps the “on-time” of the heat pulse to less than 20 milliseconds with the flux density of 400 Joules/cm², then no damage to the living cells in the underlying epidermis will be sustained, even though the ablation threshold isotherm has been moved well into the stratum corneum. In other words, by using a low flux density thermal energy source, modulated such that the “on time” is suitably short, ablation of the stratum corneum can be achieved without any damage to the adjacent cells in the underlying epidermis (see FIG. 30C). This is possible in large part due to the significantly different thermal difflisivities of these two tissues layers. That is, the stratum corneum, containing only about 10% to 20% water content, has a much lower thermal conductivity constant, 0.00123 J/(S*cm*K), than the 0.00421J/(S*cm*K) of the epidermis. This allows the temperature to build up in the stratum corneum, while maintaining a tight spatial definition, to the point at which ablation will occur.

In FIG. 32, the same simulation scenario started in the damage threshold critical point run illustrated in FIG. 31 is carried out farther in time. By leaving the heat pulse on for 58 milliseconds at the same flux density of 400 Joules/cm² within the 60 μm diameter circle of dye being heated, the pain sensory isotherm at 45° C. just enters the enervated layer of skin comprised by the dermis. In addition, the damage threshold isotherm moves significantly farther into the epidermal layer than where it was shown to be in FIG. 31. Relating this simulation to the numerous clinical studies conducted with this method, an excellent verification of the model's accuracy is obtained in that the model shows almost exactly the duration of ‘on-time’ that the heat probe can be applied to the skin before the individual feels it. In clinical tests, a controllable pulse generator was used to set the “on-time” and “off-time” of a series of light pulses applied to the topical layer of copper phthalocyanine (CPC) dye on the skin. While maintaining a constant “off-time” of 80 milliseconds, the “on-time” was gradually increased until the. subject reported a mild “pain” sensation. Without exception, all of the subjects involved in these studies, reported the first “pain” at an “on-time” of between 45 and 60 milliseconds, very close to that predicted by the model. In addition, the site-to-site variability mentioned previously as regards the sensation of “pain” was noted in these clinical studies. Accordingly, what is reported as “pain” is the point at which the first unambiguous sensation is noticeable. At one site this may be reported as pain, whereas at an adjacent site the same subject may report this as merely “noticeable.”

One element of this clinical research is the realization that even at the same site, a non-uniform pulse-train of heat pulses may work with the subject's psycho-physio logical neuro-perception to cause a genuine reduction in perceived sensation. For example, a series of shorter length heat pulses can be used to saturate the neurons in the area, momentarily depleting the neuro-transmitters available at this synaptic junction and therefore limiting the ability to send a “pain” message. This then allows a longer pulse following these short pulses to be less noticeable than if it were applied at the beginning of the sequence. Accordingly, a series of experiments was conducted with some arbitrarily created pulse trains, and the results were consistent with this hypothesis. An analogy for this situation might be found in the perception when one first steps into a very hot bath that is painful at first, but quickly becomes tolerable as one acclimates to the heat sensation.

### EXAMPLE 9

An object of this invention is to achieve a painless, micro-poration of the stratum corneum without causing any significant damage to the adjacent viable tissues. As described in the simulation illustrated in Example 8 and FIGS. 31-32, a boundary appears to exist for any given flux density of thermal energy within the ablation target spot within which the microporation can be achieved in just such a painless and non-traumatic manner. Both the in vivo and in vitro studies have shown that this is the case, and this has permitted development through empirical methods of some operational parameters that appear to work very well. The following set of simulations shows how the method works when these specific parameters are used.

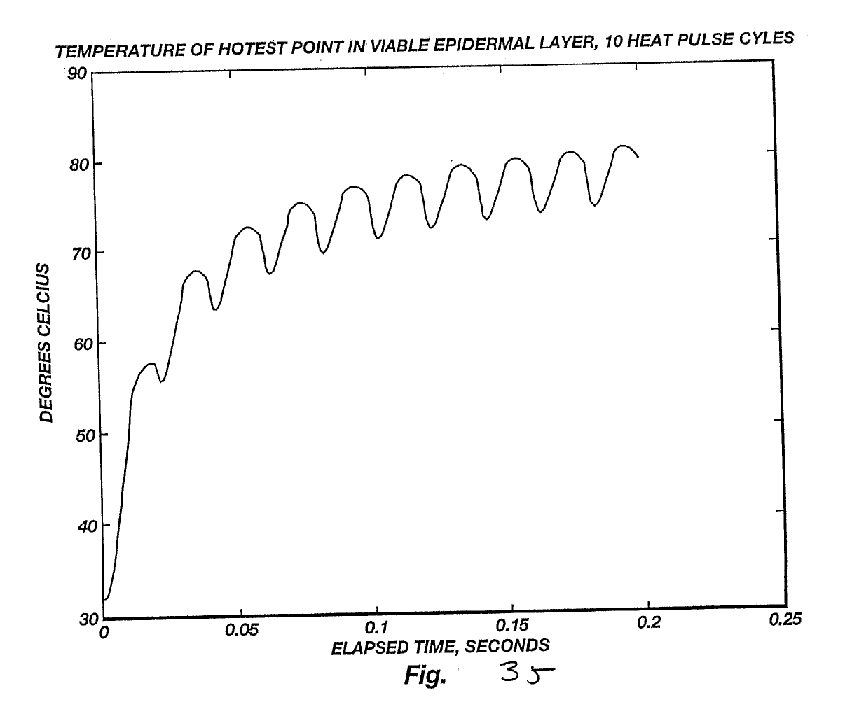

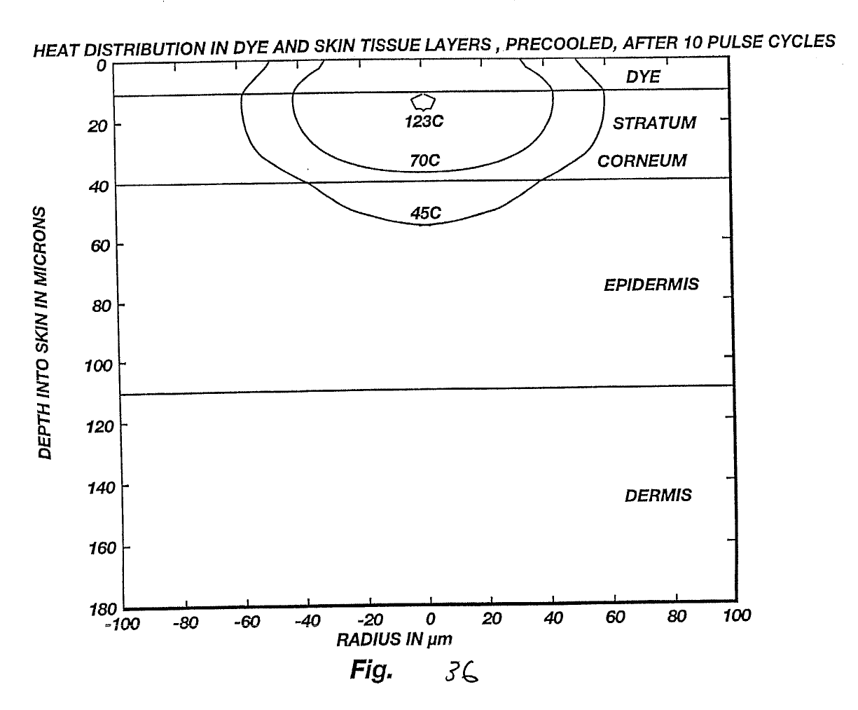

In the first case, a pulse train of ten pulses, 10 milliseconds “on-time” separated by 10 milliseconds “off-time” is applied to the CPC-covered skin. FIG. 11 shows the final temperature distribution in the skin tissues immediately after this pulse train has ended. As can be seen, the isotherms representing the three critical temperature thresholds show that stratum corneum ablation has been achieved, with no sensation present in the dermal layer nerves and very little cross-over of the damage threshold into the viable cells of the underlying epidermis. As mentioned previously, it appears that to actually do permanent cell damage, the epidermal cells must not only be heated tip to a certain point, but they also must be held at this temperature for some period of time, generally thought to be about five seconds. FIGS. 34 and 35 show the temperature of the stratum corneum and the viable epidermis, respectively, as a function of time, showing heating during the “on-time” and cooling during the “off-time” for the entire ten cycles. Relating this simulation to the in vivo studies conducted, note that in better than 90% of the poration attempts with the system parameters set to match the simulation, effective poration of the stratum corneum was achieved without pain to the subject, and in subsequent microscopic examination of the poration site several days later, no noticeable damage to the tissues was apparent. The in vitro studies conducted on whole thickness donor skin samples were also consistent with the model's prediction of behavior.

### EXAMPLE 10

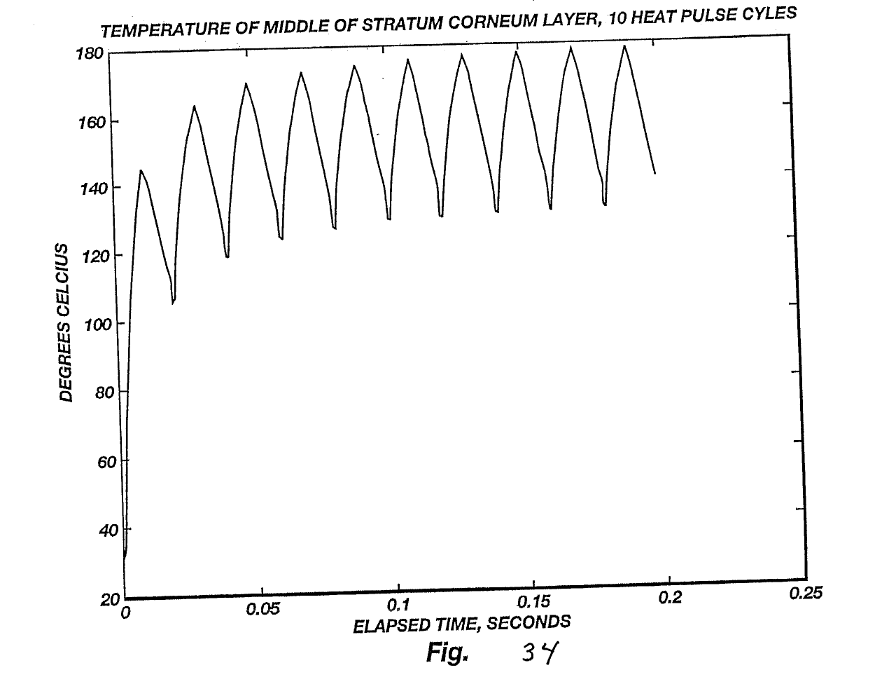

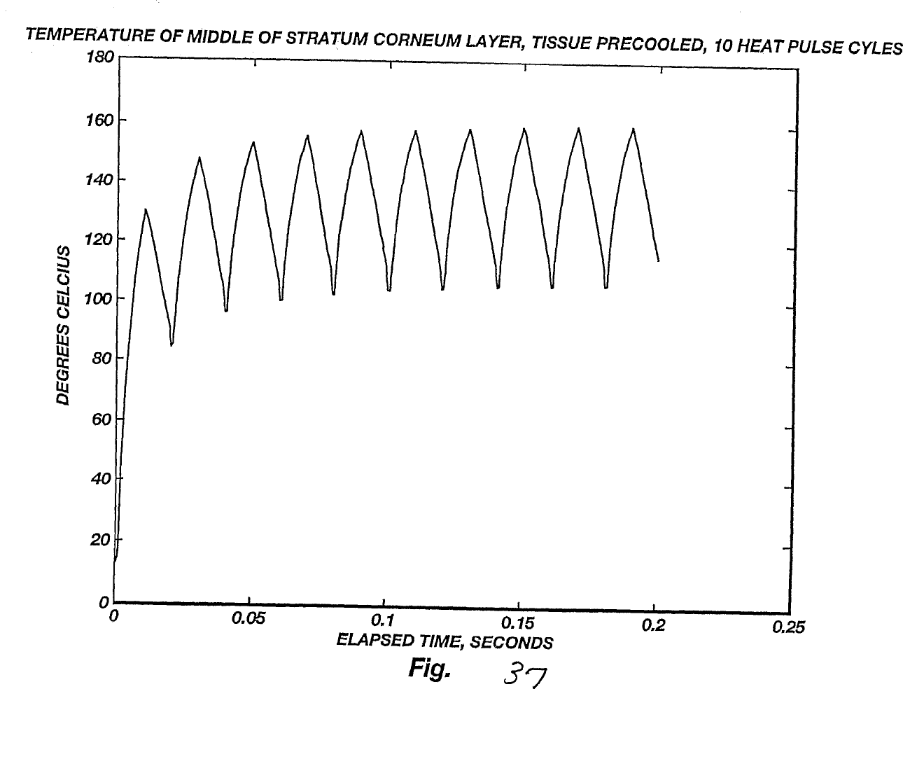

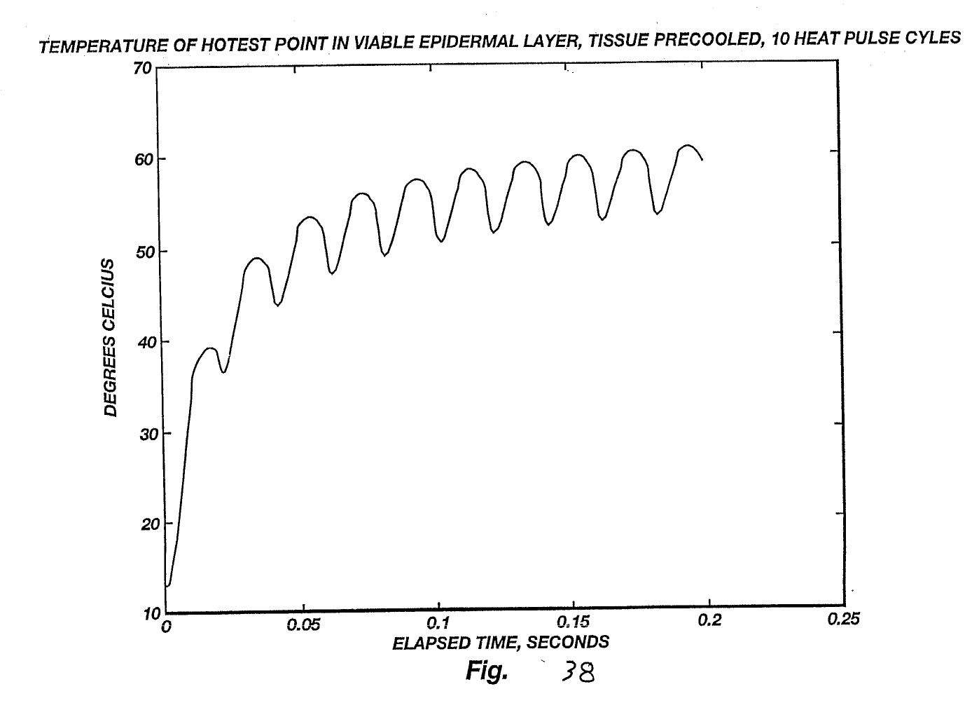

In conducting both the empirical in vivo studies, and these simulations, it appears that prechilling of the skin aids in optimizing the micro-poration process for reducing the probability of pain or damage to adjacent tissues. In practice, this can easily be achieved using a simple cold-plate placed against the skin prior to the poration process. For example, applying a Peltier cooled plate to the 1 cm diameter circle surrounding the poration target site, with the plate held at roughly 5° C. for a few seconds, significantly reduces the temperature of the tissues. A schematic illustration of an experimental device used for this purpose in the laboratory is shown in FIGS. 25A-B. By applying exactly the same ten-cycle pulse train as used in the run illustrated in Example 9, one can see, by comparing FIG. 33 to FIG. 36, FIG. 34 to FIG. 37, and FIG. 35 to FIG. 38, how much improvement can be made in the control of the temperature penetration into the skin tissues. Once again, the relatively low thermal diffusivity and specific heat of the stratum corneum as compared to the epidermis and dermis is advantageous. Once cooled, the highly hydrated tissues of the epidermis and dermis require a much larger thermal energy input to elevate their temperatures, whereas the stratum corneum, with its relatively dry makeup, can quickly be heated up to the ablation threshold.

### EXAMPLE 11

Once the basic thermal conduction mechanism of delivering the energy into the skin tissues underlying the effective painless ablation and micro-poration of the stratum corneum is understood, several different specific methods to achieve the required rapid temperature modulations of the contact point can be conceived, such as the hot wire embodiments illustrated in FIGS. 26-29.

A basic embodiment, as described herein, uses an Ohmic heating element (FIG. 26), such as the tip of a small cordless soldering iron, with a suitably sized, relatively non-reactive, wire wrapped around it with a short amount of the wire left to protrude away from the body of the heater. When electricity is applied with a constant current source, the heater will come up to some temperature and within a few seconds, achieve a steady state with the convection losses to the surrounding air. Similarly, the wire, which is a part of this thermal system, will reach a steady state such that the very tip of the wire can be raised to almost any arbitrary temperature, up to roughly 1000° C. with these types of components. The tip can be sized to give exactly the dimension micropore desired.

In the laboratory, tungsten wires with a diameter of 80 microns attached to the replaceable tip of a “WAHL” cordless soldering iron with approximately 2 mm of wire protruding from the tip have been utilized. With a thermocouple, the temperature of the tip has been measured at its steady state, and it has been noted that by varying the constant current settings, steady state temperatures of greater than 700° C. can easily be reached. To achieve the desired modulation, a low mass, fast response electromechanical actuator was coupled to the tip such that the position of the wire could be translated linearly more than 2 mm at up to a 200 Hz rate. Then, by mounting the entire apparatus on a precision stage, this vibrating tip could very controllably be brought into contact with the skin surface in a manner where it was only in contact for less than 10 milliseconds at a time, the “on-time,” while an “off-time” of arbitratily long periods could be acheived by setting the pulse generator accordingly. These in vivo studies showed that the poration could actually be achieved before the subject being porated even knew that the tip of the wire was being brought into contact with the skin.

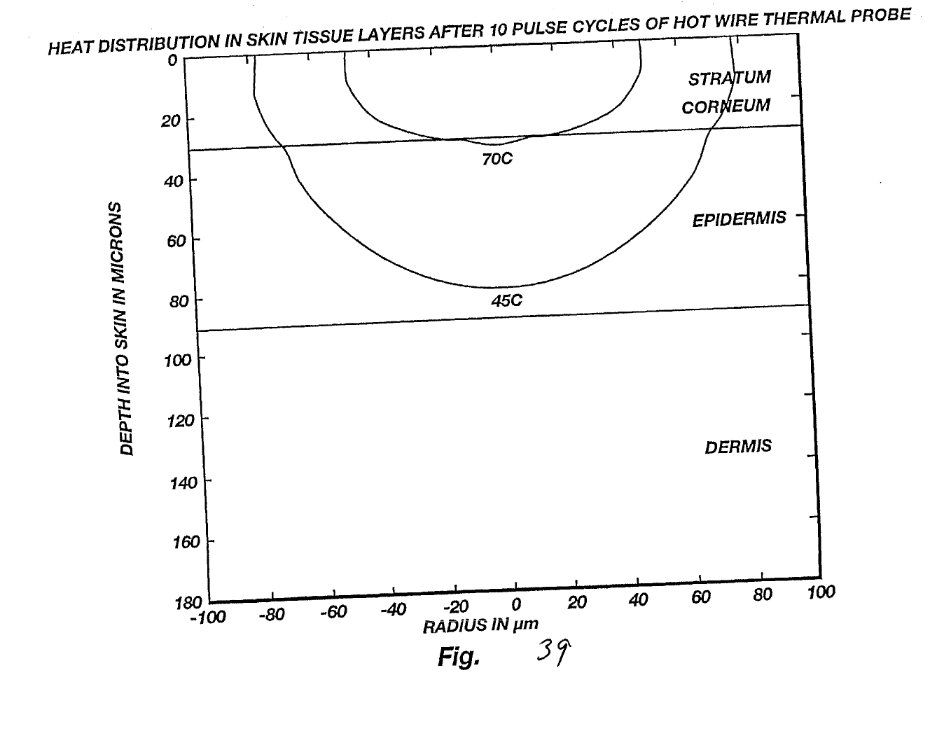

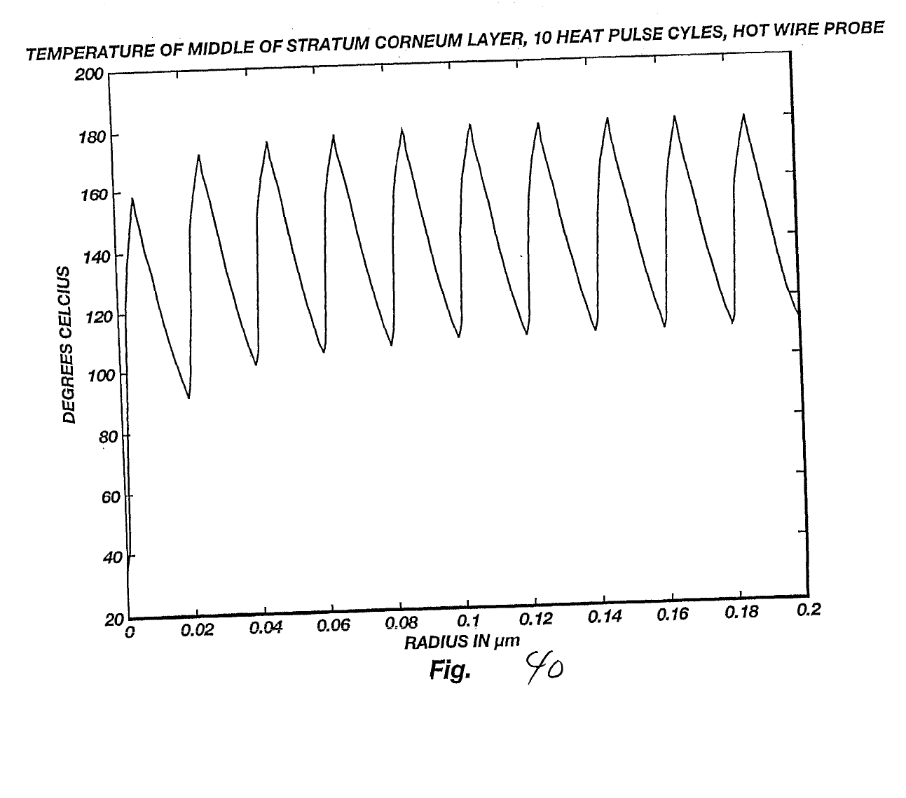

To compare the performance of this embodiment to the optically heated topical CPC dye embodiment, the following simulations were run according to the procedure of Example 8. Essentially, by only varying the initial conditions, the hot wire embodiment can be run with the identical simulation code. Because the contact with the wire occurs essentially instantly, there is no time dependent build-up of heat in the CPC dye layer and when the wire is physically removed from contact with the skin, there is a no residual heat still left on the surface as there is with the heated CPC dye layer. Also, as the wire itself defines the area targeted for ablation/micro-poration, there should be no lateral diffusion of thermal energy prior to its application to the stratum corneum. The comparative performances of the “hotwire” embodiment are shown in FIGS. 39-41.

### EXAMPLE 12

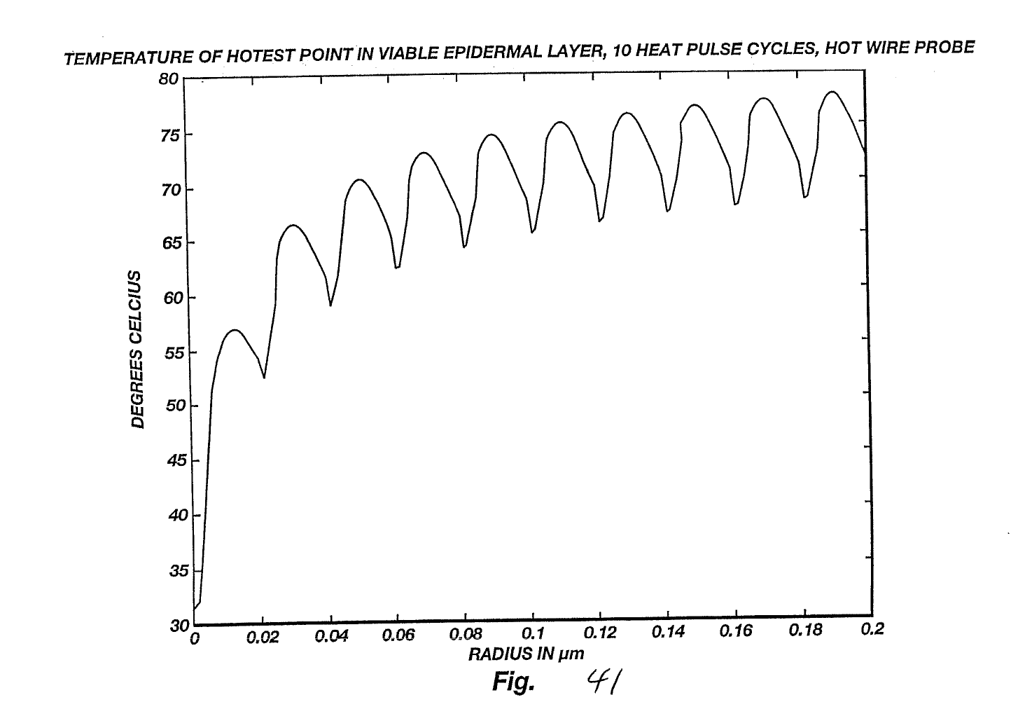

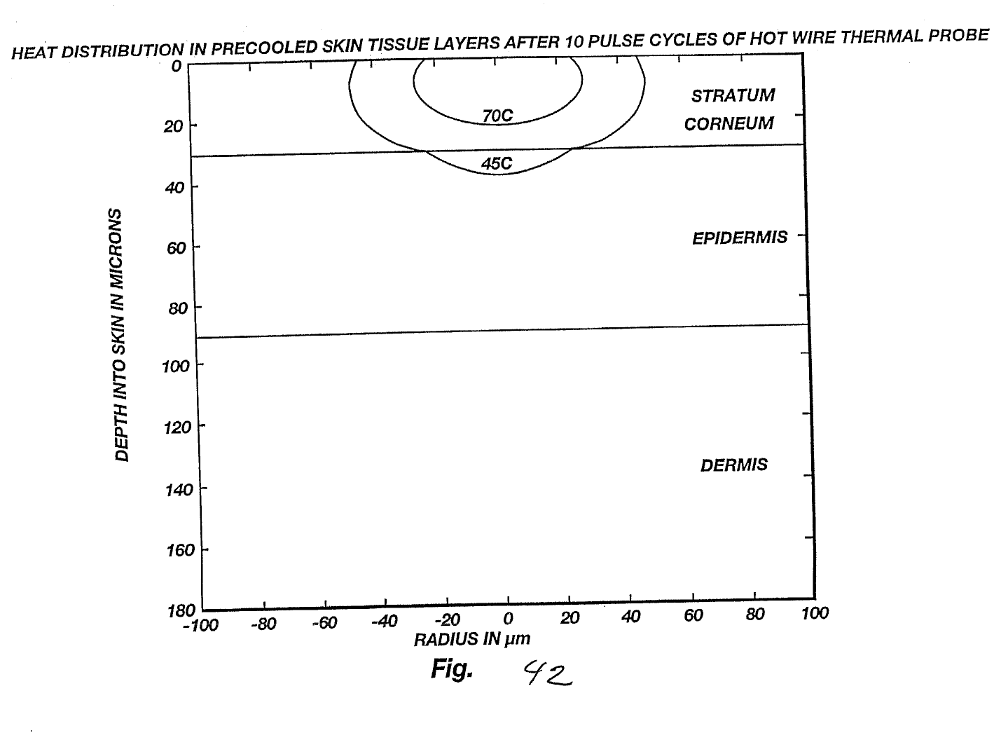

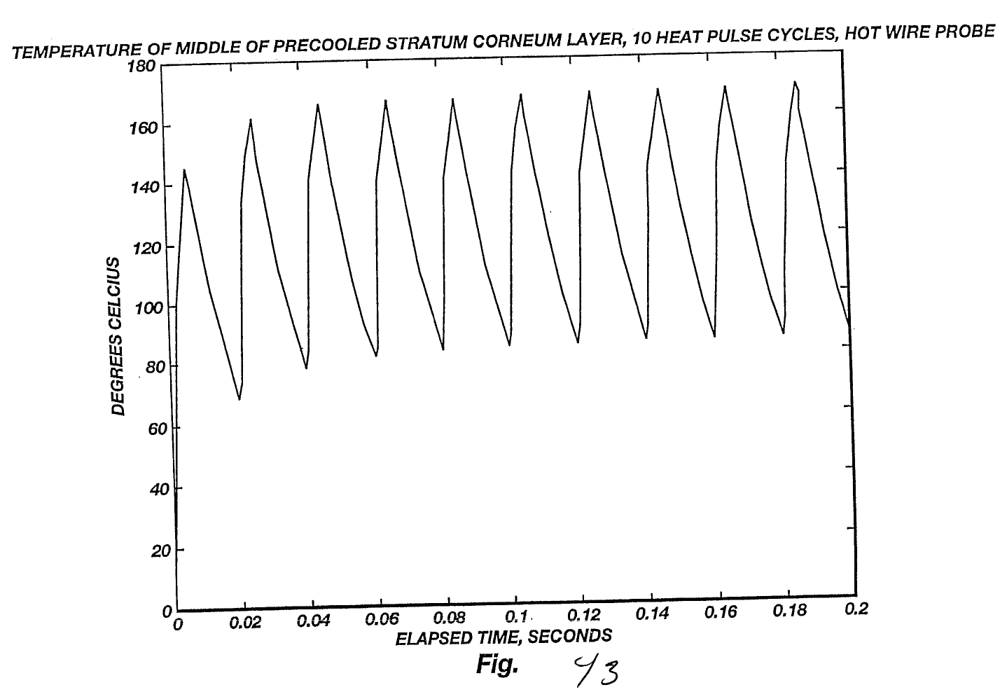

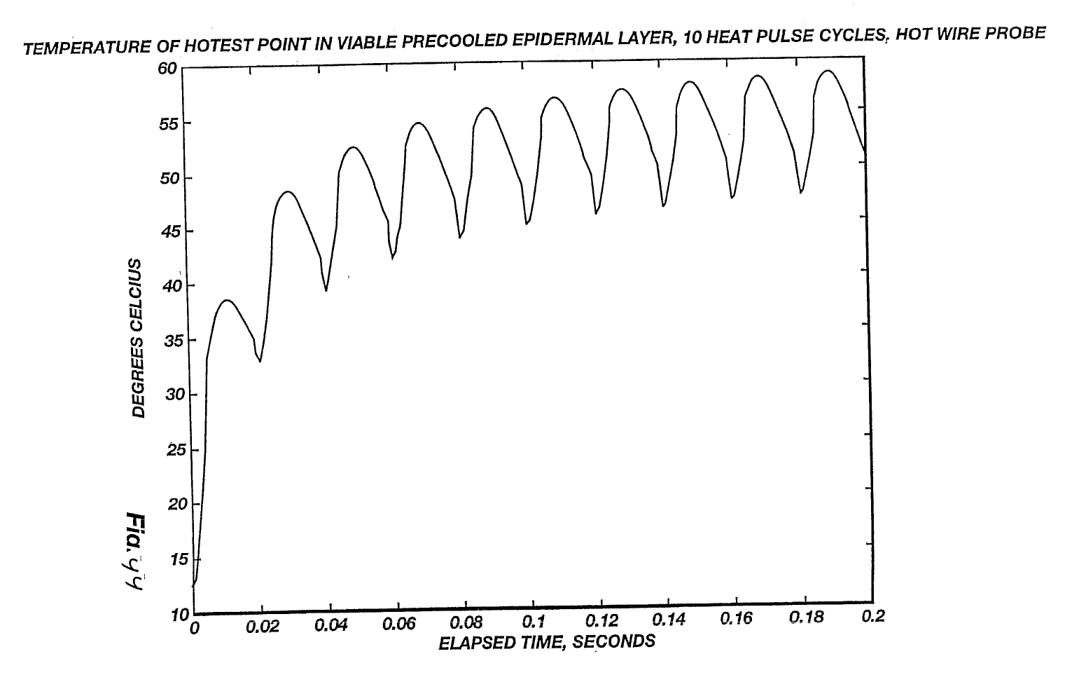

In this example, the procedure of Example 33 was followed except that the skin was pre-cooled according to the procedure of Example 32. Similarly, pre-cooling the target site yields similarly positive results with die “hot-wire” embodiment. The results of the pre-cooled simulation of the “hot-wire” approach are shown in FIGS. 42-44.

### EXAMPLE 13

As discussed in the background introduction of this disclosure, the Tankovich '803 patent appears at first glance to be similar to the presently claimed invention. In this example, the simulation model was set up with the operating parameters specified in Tankovich '803, i.e. a pulse width of 1 μs and a power level of 40,000,000 W/cm².

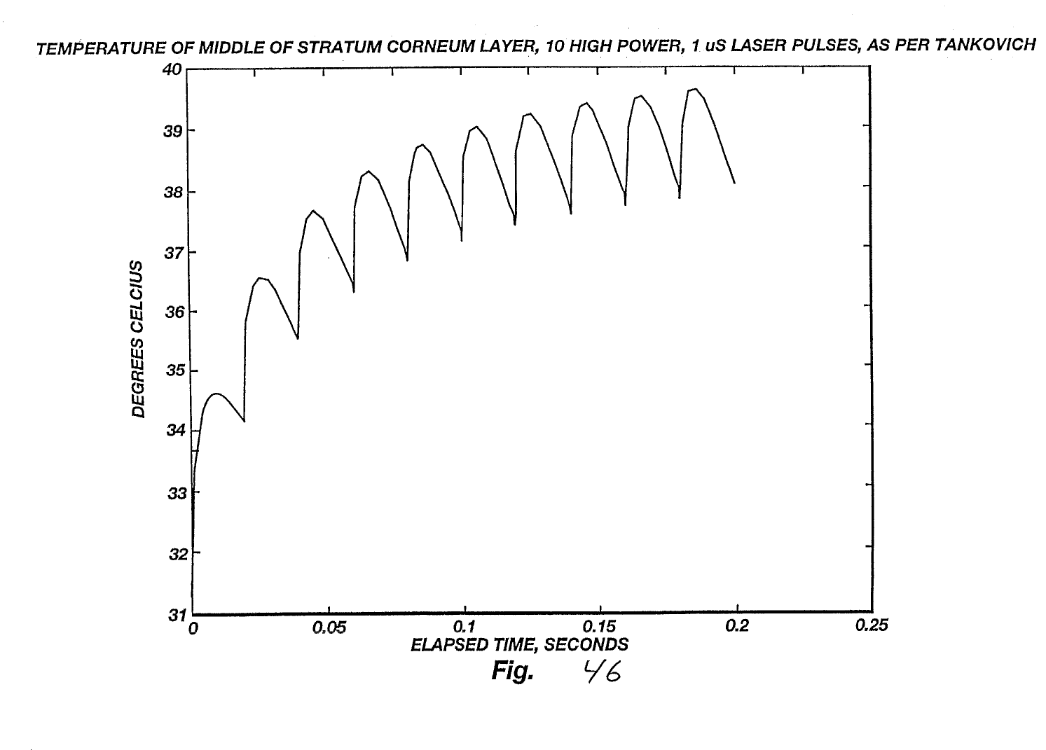

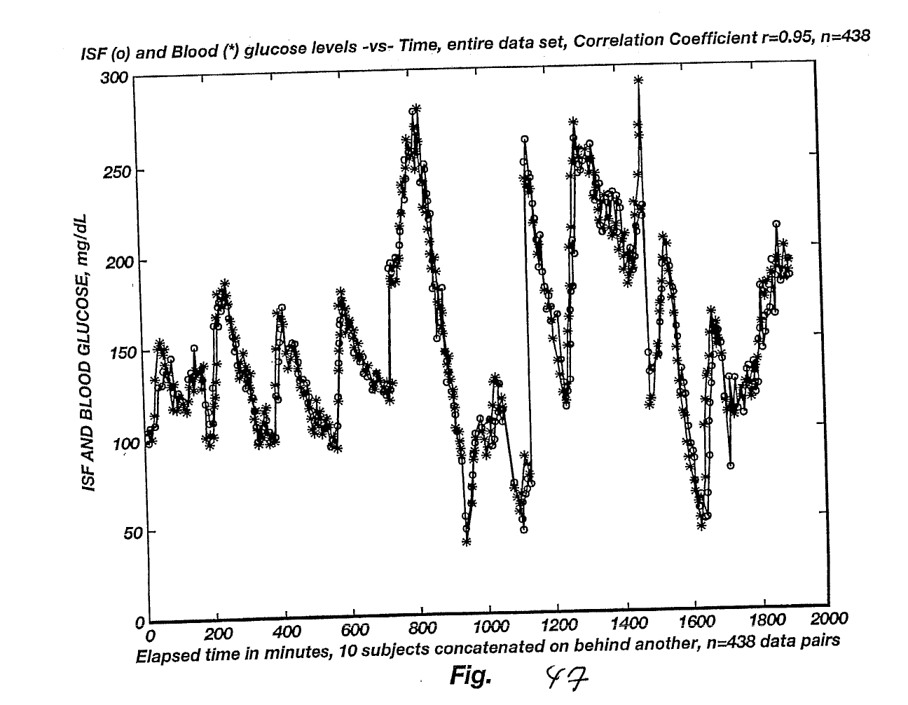

FIGS. 46 and 48 show that under these conditions no portion of the stratum corneum reaches the threshold for flash vaporization of water, 123° C., and thus no ablation/microporation of the stratum corneum occurs. In practice, applying this type of high peak power, short duration pulse to the topical dye layer merely vaporizes the dye off of the surface of the skin with no effect on the skin. This example, thus, demonstrates that the conditions specified by Tankovich '803 are inoperative in the presently claimed invention.

### EXAMPLE 14

In this example, interstitial fluid obtained after porating the skin according to the procedure of Example 6 was collected and analyzed to determine the glucose concentration thereof. Data were obtained on four non-diabetic subjects and six type I diabetic subjects undergoing a glucose load test. Subject's ages ranged from 27 to 43. The goal of the study was to examine the utility of the method for painlessly harvesting enough interstitial fluid (ISF) from the subjects to allow the ISF samples to be assayed for glucose content, and then compare these concentrations to the glucose level presenting in the subject's whole blood.

All subjects had both the blood and ISF glucose assays performed with the “ELITE” system from Miles-Bayer. All ten subjects underwent identical measurement protocols, with adjustments being made regarding the glucose load and insulin shot for those subjects with insulin dependent diabetes,

The basic design of the study was to recruit a modest number of volunteers, some with diabetes and some without diabetes, from which a series of sample pairs of ISF and whole blood were drawn every 3 to 5 minutes throughout the 3 to 4 hour duration of the study period, Both the blood and the ISF samples were assayed for glucose and the statistical relationship between the blood glucose levels and the interstitial fluid determined. To examine the hypothesized temporal lag of the ISF glucose levels as compared to the whole blood glucose levels, the study subjects were induced to exhibit a significant and dynamic change in their glucose levels. This was accomplished by having each subject fast for 12 hours prior to beginning the test and then giving the subject a glucose load after his or her baseline glucose levels have been established via a set of three fasting blood and ISF glucose levels. After the baseline levels had been established, the subjects were given a glucose load in the form of sweet juice based on the following guidelines:

i. For the control subjects, the glucose load was calculated based on a 0.75 gram glucose per pound of body weight.

ii. For the subjects with insulin dependent diabetes the glucose load was 50 grams of glucose. In addition, immediately after taking the glucose load the diabetic subjects will self inject their normal morning dose of fast acting insulin. In the case where the diAetic subject presents with fasting glucose levels above 300 mg/dL, they were asked to give themselves their insulin injection first, and the glucose load was provided after their blood glucose levels have dropped to below 120 mg/dL.

Each subject recruited was first given a complete description of the study in the “Informed Consent” document which they were required to understand and sign before they were officially enrolled into the program. Upon acceptance, they completed a medical history questionnaire. The detailed clinical procedure implemented was:

(a) Subject fasted from 9:00 p.m. the night before the study visit, consuming only water. No caffeine, cigarettes, fruit juice were allowed during this period.

(b) Subject arrived at the testing facility by 9:00 a.m. the next day.

(c) Subject was seated in a reclining chair provided for the subject to relax in throughout the study procedure.

(d) Both whole blood and ISF samples were taken at three to five minute intervals beginning upon the subject's arrival and continuing for the next three to four hours. The duration over which the data were collected was based on when the subject's blood glucose levels had returned to the normal range and stabilized after the glucose load. The ISF samples were harvested using the optical poration, ISF pumping method, described in more detail below. Each ISF sample was roughly 5 μl, by volume to ensure a good fill of the ELITE test strip. The blood samples were obtained via a conventional finger prick lancet. Both the ISF and the blood samples were immediately assayed for glucose with the ELITE home glucometer system from Miles-Bayer. To improve the estimate of the ‘true’ blood glucose levels, two separate ELITE assays were be done on each ringer stick sample.

(e) To facilitate the continued collection of the ISF from the same site throughout the entire data collection phase for a given individual, a 5 by 5 matrix of twenty five micropores was created on the subject's upper forearm, each micropore being between 50 and 80 μm across and spaced 300 microns apart. A 30 mm diameter teflon disk with a 6 mm hole in the center was attached to the subject's forearm with a pressure sensitive adhesive and positioned such that the 6 mm center hole was located over the 5 by 5 matrix of micropores. This attachment allowed a convenient method by which a small suction hose could be connected, applying a mild vacuum (10 to 12 inches of Hg) to the porated area to induce the ISF to flow out of the body through the micropores. The top of the teflon disk was fitted with a clear glass window allowing the operator to directly view the micro-porated skin beneath it. When a 5 μl bead of ISF was formed on the surface of the skin, it could easily be ascertained by visually monitoring the site through this window. This level of vacuum created a nominal pressure gradient of around 5 pounds/square inch (PSI). Without the micropores, no ISF whatsoever could be drawn from the subject's body using only the mild vacuum.

(f) After the first three sample pairs have been drawn, the subject was given a glucose load in the form of highly sweetened orange juice. The amount of glucose given was 0.75 grams per pound of body weight for the nondiabetic subjects and 50 grams for the diabetic subjects. The diabetic subjects also self administered a shot of fast acting insulin, (regular) with the dosage appropriately calculated, based on this 50 gram level of glucose concurrent with the ingestion of the glucose load. With the normal 1.5 to 2.5 hour lag between receiving an insulin shot and the maximum effect of the shot, the diabetic subjects were expected to exhibit an upwards excursion of their blood glucose levels ranging up to 300 mg/dL and then dropping rapidly back into the normal range as the insulin takes effect. The nondiabetic subjects were expected to exhibit the standard glucose tolerance test profiles, typically showing a peak in blood glucose levels between 150 mg/dL and 220 mg/dL from 45 minutes to 90 minutes after administering the glucose load, and then a rapid drop back to their normal baseline levels over the next hour or so.

(g) Following the administration of the glucose load or glucose load and insulin shot, the subjects had, samples drawn, simultaneously, of ISF and finger prick whole blood at five minute intervals for the next three to four hours. The sampling was terminated when the blood glucose levels in three successive samples indicate that the subject's glucose had stabilized.

Upon examination of the data, several features were apparent. In particular, for any specific batch of ELITE test strips, there exist a distinct shift in the output shown on the glucometer in mg/dL glucose as compared to the level indicated on the blood. An elevated reading would be expected due to the lack of hematocrit in the ISF and to the normal differences in the electrolyte concentrations between the ISF and whole blood. Regardless of the underlying reasons for this shift in output, it was determined via comparison to a reference assay that the true ISF glucose levels are linearly related to the values produced by the ELITE system, with the scaling coefficients constant for any specific batch of ELITE strips. Consequently, for the comparison of the ISF glucose levels versus the whole blood measurements, first order linear correction was applied to the ISF data as follows: ISF_{glucose}=0.606*ISF_{ELITE}+19.5.

This scaling of the output of the ELITE glucometer when used to measure ISF glucose levels, allows one to examine, over the entire data set, the error terms associated with using ISF to estimate blood glucose levels. Of course, even with no linear scaling whatsoever, the correlations between the ISF glucose values and the blood glucose levels are the same as the scaled version.

Based on the majority of the published body of literature on the subject of ISF glucose as well as preliminary data, it was originally expected that a 15 to 20 minute lag between the ISF glucose levels and the those presented in the whole blood from a ringer stick would be observed. This is not what the data showed when analyzed. Specifically, when each individual's data set is analyzed to determine the time shift required to achieve the maximum correlation between the ISF glucose levels and the blood glucose levels it was discovered that the worst case time lag for this set of subjects was only 13 minutes and the average time lag was only 6.2 minutes, with several subjects showing a temporal tracking that was almost instantaneous (about 1 minute).

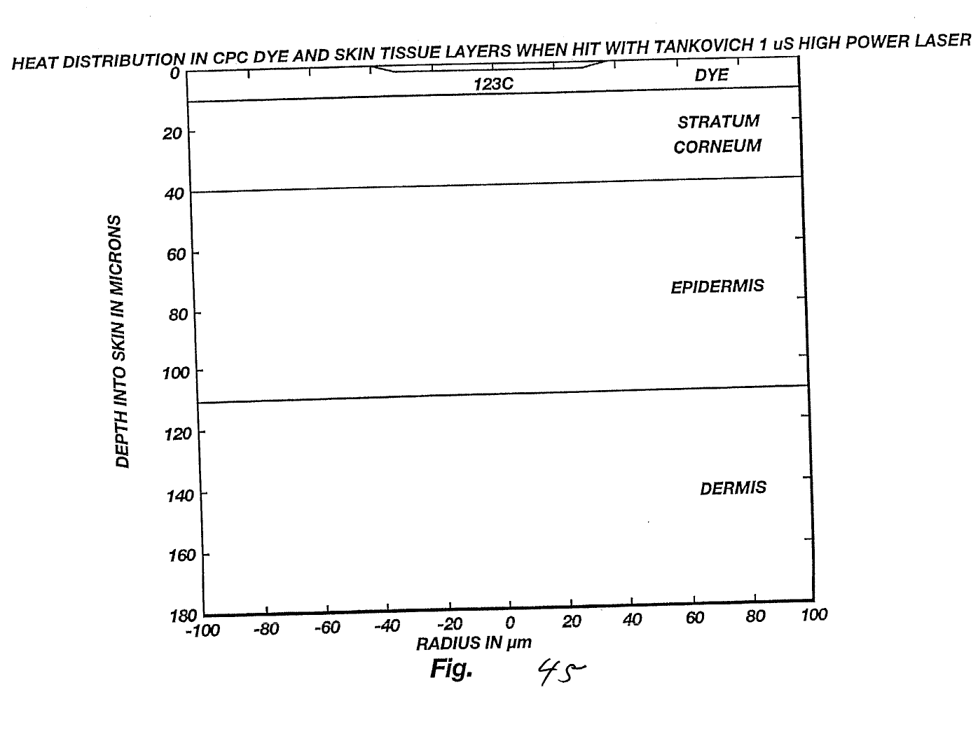

Based on the minimal amount of lag observed in this data set, the graph shown in FIG. 47 presents all ten of the glucose load tests, concatenated one after another on an extended time scale. The data are presented with no time shifting whatsoever, showing the high level of tracking between the ISF and blood glucose levels the entire clinical data set being dealt with in exactly the same manner. If the entire data set is shifted as a whole to find the best temporal tracking estimate, the correlation between the ISF and blood glucose levels peaks with a delay of two (2) minutes at an r value of r=0.97. This is only a trivial improvement from the unshifted correlation of r=0.964. Therefore, for the remainder of the analysis the ISF values are treated with no time shift imposed on them. That is, each set of blood and ISF glucose levels is dealt with as simultaneously collected data pairs.

After the unshifted Elite ISF readings bad been scaled to reflect the proportional glucose present in the ISF, it was possible to examine the error associated with these data. The simplest method for this is to assume that the average of the two ELITE finger-stick blood glucose readings is in fact the absolutely correct value, and then to merely compare the scaled ISF values to these mean blood glucose values. These data are as follows: Standard Deviation Blood-ISF, 13.4 mg/dL; Coefficient of Variance of ISF, 9.7%; Standard Deviation of the Two Elites, 8.3 mg/dL; and Coefficient of Variance of Blood (Miles), 6%.

As these data show, the blood based measurement already contains an error term. Indeed, the manufacturer's published performance data indicates that the ELITE system has a nominal Coefficient of Variance (CV) of between 5% and 7%, depending on the glucose levels and the amount of hematocrit in the blood.

An additional look at the difference term between the ISF glucose and the blood glucose is shown in the form of a scatter plot in FIG. 26. In this figure, the upper and lower bounds of the 90% confidence interval are also displayed for reference. It is interesting to note that with only two exceptions, all of the data in the range of blood glucose levels below 100 mg/dL fall within these 90% confidence interval error bars. This is important as the consequences of missing a trend towards hypoglycemia would be very significant to the diabetic user. That is, it would be much better to under-predict glucose levels in the 40 to 120 mg/dL than to over predict them.

Essentially, if one assumes that the basic assay error when the ELITE system is used on ISF is comparable to the assay error associated with the ELITE's use on whole blood, then the Deviation of the ISF glucose from the blood glucose can be described as:

ISF_{deviation}=[(ISF_{actual})²+(ISF_{actual})²]^{½}

Applying this equation to the values shown above, one can solve for the estimated ‘true’ value of the ISF error term:

ISF_{actual}=[(ISF_{deviation})²−(Blood_{actual})²]^{½}

Or, solving the equation,

ISF_{actual}=[(13.4)²−(8.3)²]^{1/2}=10.5 mg/d1.

A histogram of the relative deviation of the ISF to the blood glucose levels is shown in FIG. 27.

Drug Delivery through Pores in the Stratum Corneum

The present invention also includes method for the delivery of drugs, including drugs currently delivered transdermally, through micro-pores in the stratum corneum. In one illustrative embodiment, the delivery is achieved by placing the solution in a reservoir over the poration site. In another illustrative embodiment, a pressure gradient is used to further enhance the delivery. In still another illustrative embodiment, sonic energy is used with or without a pressure gradient to further enhance the delivery. The sonic energy can be operated according to traditional transdermal parameters or by utilizing acoustic streaming effects, which will be described momentarily, to push the delivery solution through the porated stratum corneum.

### EXAMPLE 15

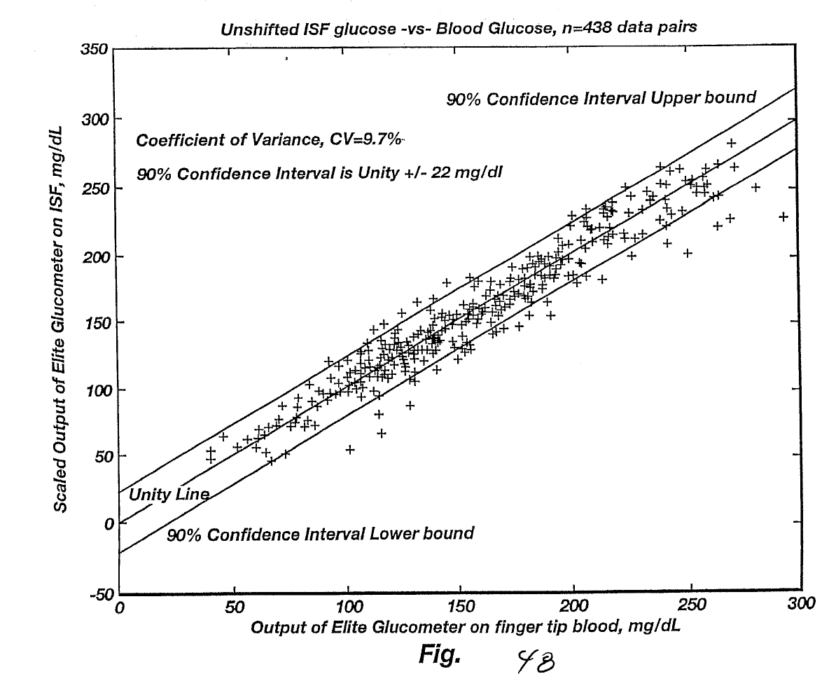

This example shows the use of stratum corneum poration for the delivery of lidocaine, a topical analgesic. The lidocaine solution also contained a chemical permeation enhancer formulation designed to enhance its passive diffusion across the stratum corneum. A drawing of an illustrative delivery apparatus 300 is shown in FIG. 50, wherein the apparatus comprises a housing 304 enclosing a reservoir 308 for holding a drug-containing solution 312. The top portion of the housing comprises an ultrasonic transducer 316 for providing sonic energy to aid in transporting the drug-containing solution through micropores; 320 in the stratum corneum 324. A port 328 in the ultrasonic transducer permits application of pressure thereto for further aiding in transporting the drug-containing solution through the micropores in the stratum corneum. The delivery apparatus is applied to a selected area of an individual's skin such that it is positioned over at least one, and preferably a plurality, of micropores. An adhesive layer 332 attached to a lower portion of the housing permits the apparatus to adhere to the skin such that the drugcontaining solution in the reservoir is in liquid communication with the micropores. Delivery of the drug through the micropores results in transport into the underlying epidermis 336 and dermis 340.

Five subjects were tested for the effectiveness of drug delivery using poration together with ultrasound. The experiment used two sites on the subjects left forearm about three inches apart, equally spaced between the thumb and upper arm. The site near the thumb will be referred to as site 1 the site furthest from the thumb will be referred to as site 2. Site 1was used as a control where the lidocaine and enhancer solution was applied using an identical delivery apparatus 300, but without any micro-poration of the stratum corneum or sonic energy. Site 2 was porated with 24 holes spaced 0.8 millimeters apart in a grid contained within a 1 cm diameter circle. The micropores in Site 2 were generated according to the procedure of Example 6. Lidocaine and low level ultrasound were applied. Ultrasound applications were made with a custom manufactured Zevex ultrasonic transducer assembly set in burst mode with 0.4 Volts peak to peak input with 1000 count bursts occurring at 10 Hz with a 65.4 kHz fundamental frequency, i.e., a pulse modulated signal with the transducer energized for 15 millisecond bursts, and then turned off for the next 85 milliseconds. The measured output of the amplifier to the transducer was 0.090 watts RMS.

After application of the lidocaine, sensation measurements were made by rubbing a 30 gauge wire across the test site. Experiments were executed on both sites, Site 1 for 10 to 12 minute duration and Site 2 for two 5 minute duration intervals applied serially to the same site. Both sites were assessed for numbness using a scale of 10 to 0, where 10 indicated no numbness and 0 indicated complete numbness as reported by the test subjects. The following summary of results is for all 5 subjects.

The control site, site 1, presented little to no numbness (scale 7 to 10) at 10 to 12 minutes. At approximately 20 minutes some numbness (scale 3) was observed at site 1 as the solution completely permeated the stratum corneum. Site 1was cleaned at the completion of the lidocaine application. Site 2 presented nearly complete numbness (scale 0 to 1) in the 1 cm circle containing the porations. Outside the 1 cm diameter circle the numbness fell off almost linearly to 1 at a 2.5 cm diameter circle with no numbness outside the 2.5 cm diameter circle. Assessment of site 2 after the second application resulted in a slightly larger totally numb circle of about 12 cm diameter with numbness failing off linearly to 1 in an irregular oval pattern with a diameter of 2 to 2.5 cm perpendicular to the forearm and a diameter of 2 to 6 cm parallel to the forearm. Outside the area no numbness was noted. A graphic representation of illustrative results obtained on a typical subject is shown in FIGS. 51A-C. FIGS. 51A and 51B show the results obtained at Site 2 (porated) after 5 and 10 minutes, respectively. FIG. 51C shows the results obtained at Site 1 (control with no poration).

Sonic Energy and Enhancers for Enhancing Transdermal Flux

The physics of sonic energy fields created by sonic transducers can be utilized in a method by which sonic frequency can be modulated to improve on flux rates achieved by other methods. As shown in FIG. 1 of U.S. Pat. No. 5,445,611, hereby incorporated herein by reference, the energy distribution of an sonic transducer can be divided into near and far fields. The near field, characterized by length N, is the zone from the first energy minimum to the last energy maximum. The zone distal to the last maximum is the far field. The near (N) field pattern is dominated by a large number of closely spaced local pressure peaks and nulls. The length of the near field zone, N, is a function of the frequency, size, and shape of the transducer face, and the speed of sound in the medium through which the ultrasound travels. For a single transducer, intensity variations within its non-nal operating range do not affect the nature of the sonic energy distribution other than in a linear fashion. However, for a system with multiple transducers, all being modulated in both frequency and amplitude, the relative intensities of separate transducers do affect the energy distribution in the sonic medium, regardless of whether it is skin or another medium.

By changing the frequency of the sonic energy by a modest amount, for example in the range of about 1 to 20%, the pattern of peaks and nulls remains relatively constant, but the length N of the near field zone changes in direct proportion to the frequency. Major changes the frequency, say a factor of 2 or more, will most likely produce a different set of resonances or vibrational modes in the transducer, causing a significantly and unpredictably different near field energy pattern. Thus, with a modest change in the sonic frequency, the complex pattern of peaks and nulls is compressed or expanded in an accordion-like manner. By selecting the direction of frequency modulation, the direction of shift of these local pressure peaks can be controlled. By applying sonic energy at the surface of the skin, selective modulation of the sonic frequency controls movement of these local pressure peaks through the skin either toward the interior of the body or toward the surface of the body. A frequency modulation from high to low drives the pressure peaks into the body, whereas a frequency modulation from low to high pulls the pressure peaks from within the body toward the surface and through the skin to the outside of the body.

Assuming typical parameters for this application of, for example, a 1.27 cm diameter sonic transducer and a nominal operating frequency of 10 MHz and an acoustic impedance similar to that of water, a frequency modulation of 1 MHz produces a movement of about 2.5 mm of the peaks and nulls of the near field energy pattern in the vicinity of the stratum corneum. From the perspective of tratisdermal and/or transmucosal withdrawal of analytes, this degree of action rovides access to the area well below the stratum corneum and even the epidermis, dermis, and other tissues beneath it. For any given transducer, there may be an optimal range of frequencies within which this frequency modulation is most effective.

The flux of a drug or analyte across the skin can also be increased by changing either the resistance (the diffusion coefficient) or the driving force (the gradient for diffusion). Flux can be enhanced by the use of so-called penetration or chemical enhancers.

Chemical enhancers are comprised of two primary categories of components, i.e., cell envelope disordering compounds and solvents or binary systems containing both cell-envelope disordering compounds and solvents.

Cell envelope disordering compounds are known in the art as being useful pharmaceutical preparations and function also in analyte withdrawal through the skin. These compounds are thought to assist in skin penetration by disordering the lipid structure of the stratum corneum cell-envelopes. A comprehensive list of these compounds is described in European Patent Application 43,738, published Jun. 13, 1982, which is incorporated herein by reference. It is believed that any cell envelope disordering compound is useful for purposes of this invention. Suitable solvents include water; diols, such as propylene glycol and glycerol; mono-alcohols, such as ethanol, propanol, and higher alcohols; DMSO; dimethylformamide; N,N-dimethylacetamide; 2-pyrrolidone; N-(2-hydroxyethyl) pyrrolidone, N-methylpyrrolidone, 1-dodecylazacycloheptan-2-one and other n-substituted-alkyl-azacycloalkyl-2-ones(azones) and the like.

U.S. Pat. No. 4,537,776, Cooper, issued Aug. 27, 1985, contains an excellent summary of prior art and background information detailing the use of certain binary systems for permeant enhancement. Because of the completeness of that disclosure, the information and terminology utilized therein are incorporated herein by reference.

Similarly, European Patent Application 43,738, referred to above, teaches using selected diols as solvents along with a broad category of cell-envelope disordering compounds for delivery of lipophilic pharmacologically-active compounds. Because of the detail in disclosing the cellenvelope disordering compounds and the diols, this disclosure of European Patent Application 43,738 is also incorporated herein by reference.

A binary system for enhancing metoclopramide penetration is disclosed in UK Patent Application GB 2,153,223 A, published Aug. 21, 1985, and consists of a monovalent alcohol ester of a C8-32 aliphatic monocarboxylic acid (unsaturated and/or branched if C18-32) or a C6-24 aliphatic monoalcohol (unsaturated and/or branched if C14-24) and an N-cyclic compound such as 2-pyrrolidone, N-methylpyrrolidone and the like.

Combinations of enhancers consisting of diethylene glycol monoethyl or monomethyl etherwith propylene glycol monolaurate and methyl laurate are disclosed in U.S. Pat. No. 4,973,468 as enhancing the transdermal delivery of steroids such as progestogens and estrogens. A dual enhancer consisting of glycerol monolaurate and ethanol for the transdermal delivery of drugs is shown in U.S. Pat. No. 4,820,720. U.S. Pat. No. 5,006,342 lists numerous enhancers for transdermal drug administration consisting of fatty acid esters or fatty alcohol ethers of C₂ to C₄ alkanediols, where each fatty acid/alcohol portion of the ester/ether is of about 8 to 22 carbon atoms. U.S. Pat. No. 4,863,970 shows penetration-enhancing compositions for topical application comprising an active permeant contained in a penetration-enhancing vehicle containing specified amounts of one or more cell-envelope disordering compounds such as oleic acid, oleyl alcohol, and glycerol esters of oleic acid; a C₂ or C₃ alkanol and an inert diluent such as water.

Other chemical enhancers, not necessarily associated with binary systems include DMSO or aqueous solutions of DMSO such as taught in Herschler, U.S. Pat. No. 3,551,554; Herschler, U.S. Pat. No. 3,711,602; and Herschler, U.S. Pat. No. 3,711,606, and the azones (n-substituted-alkylazacycloalkyl-2-ones) such as noted in Cooper, U.S. Pat. No. 4,557,943.

Some chemical enhancer systems may possess negative side effects such as toxicity and skin irritation. U.S. Pat. No. 4,855,298 discloses compositions for reducing skin irritation caused by chemical enhancer containing compositions having skin irritation properties with an amount of glycerin sufficient to provide an anti-irritating effect.

Because the combination of microporation of the stratum corneum and the application of sonic energy accompanied by the use of chemical enhancers can result in an improved rate of analyte withdrawal or permeant delivery through the stratum corneum, the specific carrier vehicle and particularly the chemical enhancer utilized can be selected from a long list of prior art vehicles some of which are mentioned above and incorporated herein by reference. To specifically detail or enumerate that which is readily available in the art is not thought necessary. The invention is not drawn to the use of chemical enhancers per se and it is believed that all chemical enhancers, useful in the delivery of drugs through the skin, will function with dyes in optical microporation and also with sonic energy in effecting measurable withdrawal of analytes from beneath and through the skin surface or the delivery of permeants or drugs through the skin surface.

### EXAMPLE 16

Modulated sonic energy and chemical enhancers were tested for their ability to control transdermal flux on human cadaver skin samples. In these tests, the epidermal membrane had been separated from the human cadaver whole skin by the heat-separation method of Example 1. The epidermal membrane was cut and placed between two halves of the permeation cell with the stratum corneum facing either the upper (donor) compartment or lower (receiver) compartment. Modified Franz cells were used to hold the epidermis, as shown in FIG. 2 of U.S. Pat. No. 5,445,611. Each Franz cell consists of an upper chamber and a lower chamber held together with one or more clamps. The lower chamber has a sampling port through which materials can be added or removed. A sample of stratum corneum is held between the upper and lower chambers when they are clamped together. The upper chamber of each Franz cell is modified to allow an ultrasound transducer to be positioned within 1 cm of the stratum corneum membrane. Methylene blue solution was used as an indicator molecule to assess the permeation of the stratum corneum. A visual record of the process and results of each experiment was obtained in a time stamped magnetic tape format with a video camera and video cassette recorder (not shown). Additionally, samples were withdrawn for measurement with an absorption spectrometer to quantitate the amount of dye which had traversed the stratum corneum membrane during an experiment. Chemical enhancers suitable for use could vary over a wide range of solvents and/or cell envelope disordering compounds as noted above. The specific enhancer utilized was: ethanol/glycerol/water/glycerolmonooleate/methyl laurate in 50/30/15/2.5/2.5 volume ratios. The system for producing and controlling the sonic energy included a programmable 0-30 MHz arbitrary waveform generator (Stanford Reserach Systems Model DS345), a 20 watt 0-30 MHz amplifier, and two unfocused ultrasound immersion transducers having peak resonances at 15 and 25 MHz, respectively. Six cells were prepared simultaneously for testing of stratum corneum samples from the same donor. Once the stratum corneum samples were installed, they were allowed to hydrate with distilled water for at least 6 hours before any tests were done.

### EXAMPLE 17

Effects of Sonic Energy without Chemical Enhancers

As stated abovein Example 16, the heat-separated epidermis was placed in the Franz cells with the epidermal side facing up, and the stratum corneum side facing down, unless noted otherwise. The lower chambers were filled with distilled water, whereas the upper chambers were filled with concentrated methylene blue solution in distilled water.

Heat Separated Epidermis: Immediately after filling the upper chambers with methylene blue solution, sonic energy was applied to one of the cells with the transducer fully immersed. This orientation would correspond, for example, to having the transducer on the opposite side of a fold of skin, or causing the sonic energy to be reflected off a reflector plate similarly positioned and being used to “push” analyte out of the other side of the fold into a collection device. The sonic energy setting was initially set at the nominal operating frequency of 25 MHz with an intensity equivalent to a 20 volt peak-to-peak (P-P) input wave form. This corresponds to roughly a 1 watt of average input power to the transducer and similarly, assuming the manufacturer's nominal value for conversion efficiency of 1% for this particular transducer, a sonic output power of around 0.01 watts over the 0.78 cm² surface of the active area or a sonic intensity of 0.13 watts/cm². Three other control cells had no sonic energy applied to them. After 5 minutes the sonic energy was turned off. No visual indication of dye flux across the stratum corneum was observed during this interval in any of the cells, indicating levels less than approximately 0.0015% (v/v) of dye solution in 2 ml of receiver medium.

Testing of these same 3 control cells and 1 experimental cell was continued as follows. The intensity of sonic energy was increased to the maximum possible output available from the driving equipment of a 70 volt peak-to-peak input 12 watts average power input or (≈0.13 watts/cm²) of sonic output intensity. Also, the frequency was set to modulate or sweep from 30 MHz to 10 MHz. This 20 MHz sweep was performed ten times per second, i.e., a sweep rate of 10 Hz. At these input power levels, it was necessary to monitor the sonic energy transducer to avoid overheating. A contact thermocouple was applied to the body of the transducer and power was cycled on and off to maintain maximum temperature of the transducer under 42° C. After about 30 minutes of cycling maximum power at about a 50% duty cycle of Iminute on and minute off, there was still no visually detectable permeation of the stratum corneum by the methylene blue dye.

A cooling water jacket was then attached to the sonic energy transducer to permit extended excitation at the maximum energy level. Using the same 3 controls and 1 experimental cell, sonic energy was applied at maximum power for 12 hours to the experimental cell. During this time the temperature of the fluid in the upper chamber rose to only 35° C., only slightly above the approximately 31° C. normal temperature of the stratum corneum in vivo. No visual evidence of dye flux through the stratum corneum was apparent in any of the four cells after 12 hrs of sonic energy applied as described above.

### EXAMPLE 18

Effects of Sonic Energy without Chemical Enhancers

Perforated Stratum Corneum: Six cells were prepared as described above in Example 16. The clamps holding the upper and lower chambers of the Franz cells were tightened greater than the extent required to normally seal the upper compartment from the lower compartment, and to the extent to artificially introduce perforations and “pinholes” into the heat-separated epidermal samples. When dye solution was added to the upper chamber of each cell, there were immediate visual indications of leakage of dye into the lower chambers through the perforations formed in the stratum corneum. Upon application of sonic energy to cells in which the stratum corneum was so perforated with small “pinholes,” a rapid increase in the transport of fluid through a pinhole in the stratum corneum was observed. The rate of transport of the indicator dye molecules was directly related to whether the sonic energy was applied or not. That is, application of the sonic energy caused an immediate (lag time approximately <0.1 second) pulse of the indicator molecules through the pinholes in the stratum corneum. This pulse of indicator molecules ceased immediately upon turning off of the sonic energy (a shutoff lag of approximately <0.1 second). The pulse could be repeated as described.

### EXAMPLE 19

Effects of Sonic Energy and Chemical Enhancers

Two different chemical enhancer formulations were used. Chemical Enhancer One or CE1 was an admixture of ethanol/glycerol/water/glycerol monooleate/methyl laurate in a 50/30/15/2.5/2.5 volume ratio. These are components generally regarded as safe, i.e. GRAS, by the FDA for use as pharmaceutical excipients. Chemical Enhancer Two or CE2 is an experimental formulation shown to be very effective in enhancing transdermal drug delivery, but generally considered too irritating for long term transdermal delivery applications. CE2 contained ethanol/glycerol/water/lauradone/methyllaurate in the volume ratios 50/30/15/2.5/2.5. Lauradone is the lauryl (dodecyl) ester of 2-pyrrolidone-5-carboxylic acid (“PCA”) and is also referred to as lauryl PCA.

Six Franz cells were set up as before (Example 16) except that the heat separated epidermis was installed with the epidermal layer down, i.e., stratum corneum side facing up. Hydration was established by exposing each sample to distilled water overnight. To begin the experiment, the distilled water in the lower chambers was replaced with methylene blue dye solution in all six cells. The upper chambers were filled with distilled water and the cells were observed for about 30 minutes confirming no passage of dye to ensure that no pinhole perforations were present in any of the cells. When none were found, the distilled water ill the upper chambers was removed from four of the cells. The other two cells served as distilled water controls. The upper chambers of two of the experimental cells were then filled with CE1 and the other two experimental cells were filled with CE2.

Sonic energy was immediately applied to one of the two CE2 cells. A 25 MHz transducer was used with the frequency sweeping every 0. 1 second from 10 MHz to 30 MHz at maximum intensity of ≈0. 13 watts/cm². After 10-15 minutes of sonic energy applied at a 50% duty cycle, dye flux was visually detected. No dye flux was detected in the other five cells.

Sonic energy was then applied to one of the two cells containing CE1 at the same settings. Dye began to appear in the upper chamber within 5 minutes. Thus, sonic energy together with a chemical enhancer significantly increased the transdermal flux rate of a marker dye through the stratum corneum, as well as reduced the lag time.

### EXAMPLE 20

Effects of Sonic Energy and Chemical Enhancers

Formulations of the two chemical enhancers, CE1 and CE2, were prepared minus the glycerin and these new formulations, designated CE1MG and CE2MG, were tested as before. Water was substituted for glycerin so that the proportions of the other components remained unchanged. Three cells were prepared in modified Franz cells with the epidermal side of the heat separated epidermis samples facing toward the upper side of the chambers. These samples were then hydrated in distilled water for 8 hours. After the hydration step, the distilled water in the lower chambers was replaced with either CE1MG or CE2MG and the upper chamber was filled with the dye solution. Sonic energy was applied to each of the three cells sequentially.

Upon application of pulsed, frequency-modulated sonic energy for a total duration of less than 10 minutes, a significant increase in permeability of the stratum corneum samples was observed. The permeability of the stratum corneum was altered relatively uniformly across the area exposed to both the chemical enhancer and sonic energy. No “pinhole” perforations through which the dye could traverse the stratum corneum were observed. The transdermal flux rate was instantly controllable by turning the sonic energy on or off. Turning the sonic energy off appeared to instantly reduce the transdermal flux rate such that no dye was visibly being actively transported through the skin sample; presumably the rate was reduced to that of passive diffusion. Turning the sonic energy on again instantly resumed the high level flux rate. The modulated mode appeared to provide a regular pulsatile increase in the transdermal flux rate at the modulated rate. When the sonic energy was set to a constant frequency, the maximum increase in transdermal flux rate for this configuration seemed to occur at around 27 MHz.

Having obtained the same results with all three samples, the cells were then drained of all fluids and flushed with distilled water on both sides of the stratum corneum. The lower chambers were then immediately filled with distilled water and the upper chambers were refilled with dye solution. The cells were observed for 30 minutes. No holes in the stratum corneum samples were observed and no large amount of dye was detected in the lower chambers. A small amount of dye became visible in the lower chambers, probably due to the dye and enhancer trapped in the skin samples from their previous exposures. After an additional 12 hours, the amount of dye detected was still very small.

### EXAMPLE 21

Effects of Sonic Energy and Chemical Enhancers

Perforated Stratum Corneum: Three cells were prepared with heat-separated epidermis samples with the epidermal side facing toward the upper side of the chamber from the same donor as in Example 16. The samples were hydrated for 8 hours and then the distilled water in the lower chambers was replaced with either CE1MG or CE2MG. The upper chambers were then filled with dye solution. Pinhole perforations in the stratum corneum samples permitted dye to leak through the stratum corneum samples into the underlying enhancer containing chambers. Sonic energy was applied. Immediately upon application of the sonic energy, the dye molecules were rapidly pushed through the pores. As shown above, the rapid flux of the dye through the pores was directly and immediately correlated with the application of the sonic energy.

### EXAMPLE 22

Effects of Sonic Energy and Chemical Enhancers

A low cost sonic energy transducer, TDK #NB-58S-01 (TDK Corp.), was tested for its capability to enhance transdermal flux rates. The peak response of this transducer was determined to be about 5.4 MHz with other local peaks occurring at about 7 MHz, 9 MHz, 12.4 MHz, and 16 MHz.

This TDK transducer was then tested at 5.4 MHz for its ability to enhance transdermal flux rate in conjunction with CE1MG. Three cells were set up with the epidermal side facing the lower chamber, then the skin samples were hydrated for 8 hrs. The dye solution was placed in the lower chamber. The transducer was placed in the upper chamber immersed in CE1MG. Using swept frequencies from 5.3 to 5.6 MHz as the sonic energy excitation, significant quantities of dye moved through the stratum corneum and were detected in the collection well of the cell in 5 minutes. Local heating occurred, with the transducer reaching a temperature of 48° C. In a control using CE1MG without sonic energy, a 24 hour exposure yielded less dye in the collection well than the 5 minute exposure with sonic energy.

This example demonstrates that a low cost, low frequency sonic energy transducer can strikingly affect transdermal flux rate when used in conjunction with an appropriate chemical enhancer. Although higher frequency sonic energy will theoretically concentrate more energy in the stratum corneum, when used with a chemical enhancer, the lower frequency modulated sonic energy can accelerate the transdermal flux rate to make the technology useful and practical.

### EXAMPLE 23

Demonstration of molecule migration across human skin: Tests with the TDK transducer and CE1MG described above were repeated at about 12.4 MHz, one of the highest local resonant peaks for the transducer, with a frequency sweep at a 2 Hz rate from 12.5 to 12.8 MHz and an sonic energy density less than 0.1 W/cm². The epidermal side of the heat-separated epidermis was facing down, the dye solution was in the lower chamber, and the enhancer solution and the sonic energy were placed in the upper chamber. Within 5 minutes a significant amount of dye had moved across the stratum corneum into the collection well. Ohmic heating in the transducer was significantly less than with the same transducer being driven at 5.4 MHz, causing an increase in temperature of the chemical enhancer to only about 33° C.

Even at these low efficiency levels, the results obtained with CE1MG and sonic energy from the TDK transducer were remarkable in the monitoring direction. FIGS. 3A and 3B of U.S. Pat. No. 5,445,611 show plots of data obtained from three separate cells with the trandermal flux rate measured in the monitoring direction, Even at the 5 minute time point, readily measurable quantities of the dye were present in the chemical enhancer on the outside of the stratum corneum, indicating transport from the epidermal side through the stratum corneum to the “outside” area of the skin sample.

To optimize the use of the sonic energy or the sonic energy/chemical enhancer approach for collecting and monitoring analytes from the body, means for assaying the amount of analyte of interest are required. An assay system that takes multiple readings while the unit is in the process of withdrawing analytes by sonic energy with or without chemical enhancers makes it unnecessary to standardize across a broad population base and normalize for different skin characteristics and flux rates. By plotting two or more data points in time as the analyte concentration in the collection system is increasing, a curve-fitting algorithm can be applied to determine the parameters describing the curve relating analyte withdrawal or flux rate to the point at which equilibrium is reached, thereby establishing the measure of the interval concentration. The general form of this curve is invariant from one individual to another; only the parameters change. Once these parameters are established, solving for the steady state solution (i.e., time equals infinity) of this function, i.e., when full equilibrium is established, provides the concentration of the analyte within the body. Thus, this approach permits measurements to be made to the desired level of accuracy in the same amount of time for all members of a population regardless of individual variations in skin permeability.

Several existing detection techniques currently exist that are adaptable for this application. See, D. A. Christensen, in 1648 Proceedings of Fiber Optic, Medical and Fluorescent Sensors and Applications 223-26 (1992). One method involves the use of a pair of optical fibers that are positioned close together in an approximately parallel manner. One of the fibers is a source fiber, through which light energy is conducted. The other fiber is a detection fiber connected to a photosensitive diode. When light is conducted through the source fiber, a portion of the light energy, the evanescent wave, is present at the surface of the fiber and a portion of this light energy is collected by the detection fiber. The detection fiber conducts the captured evanescent wave energy to the photosensitive diode which measures it. The fibers are treated with a binder to attract and bind the analyte that is to be measured. As analyte molecules bind to the surface (such as the analyte glucose binding to immobilized lectins such as concanavalin A, or to immobilized anti-glucose antibodies) the amount of evanescent wave coupling between the two fibers is changed and the amount of energy captured by the detection fiber and measured by the diode is changed as well. Several measurements of detected evanescent wave energy over short periods of time support a rapid determination of the parameters describing the equilibrium curve, thus making possible calculation of the concentration of the analyte within the body. The experimental results showing measurable flux within 5 minutes (FIGS. 3A and 3B of U.S. Pat. No. 5,445,611) with this system suggest sufficient data for an accurate final reading are collected within 5 minutes.

In its most basic embodiment, a device that can be utilized for the application of sonic energy and collection or analyte comprises an absorbent pad, either of natural or synthetic material, which serves as a reservoir for the chemical enhancer, if used, and for receiving the analyte from the skin surface. The pad or reservoir is held in place, either passively or aided by appropriate fastening means, such as a strap or adhesive tape, on the selected area of skin surface.

An sonic energy transducer is positioned such that the pad or reservoir is between the skin surface and the transducer, and held in place by appropriate means. A power supply is coupled to the transducer and activated by switch means or any other suitable mechanism. The transducer is activated to deliver sonic energy modulated in frequency, phase or intensity, as desired, to deliver the chemical enhancer, if used, from the reservoir through the skin surface followed by collection of the analyte from the skin surface into the reservoir. After the desired fixed or variable time period, the transducer is deactivated. The pad or reservoir, now containing the analyte of interest, can be removed to quantitate the analyte, for example, by a laboratory utilizing any number of conventional chemical analyses, or by a portable device.

Alternately, the mechanism for quantitating the analyte can be built into the device used for collection of the analyte, either as an integral portion of the device or as an attachment. Devices for monitoring an analyte are described in U.S. Pat. No. 5,458,140, which is incorporated herein by reference.

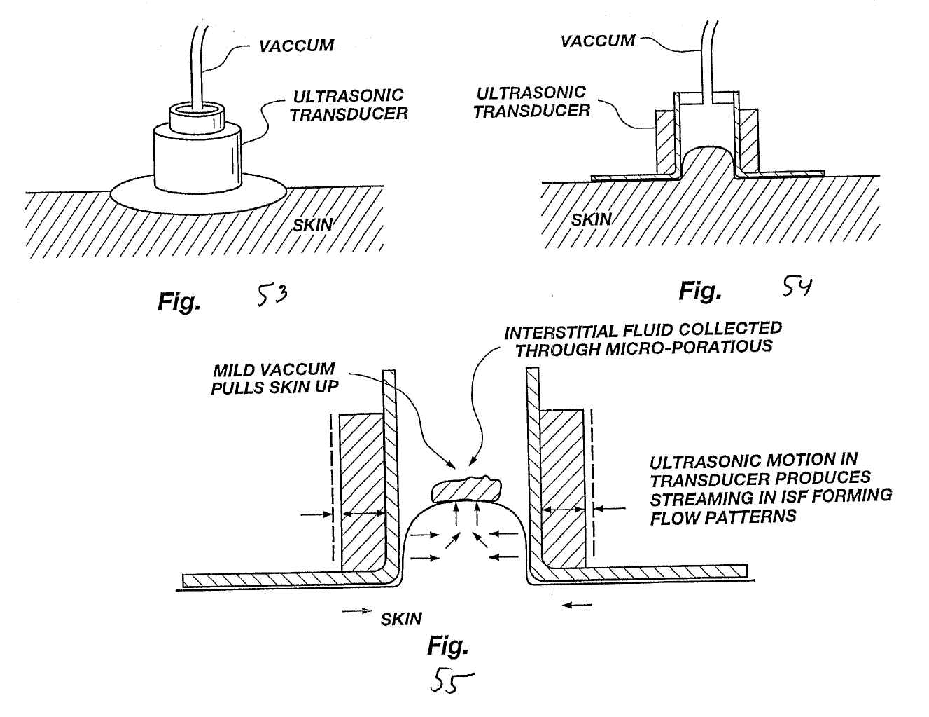

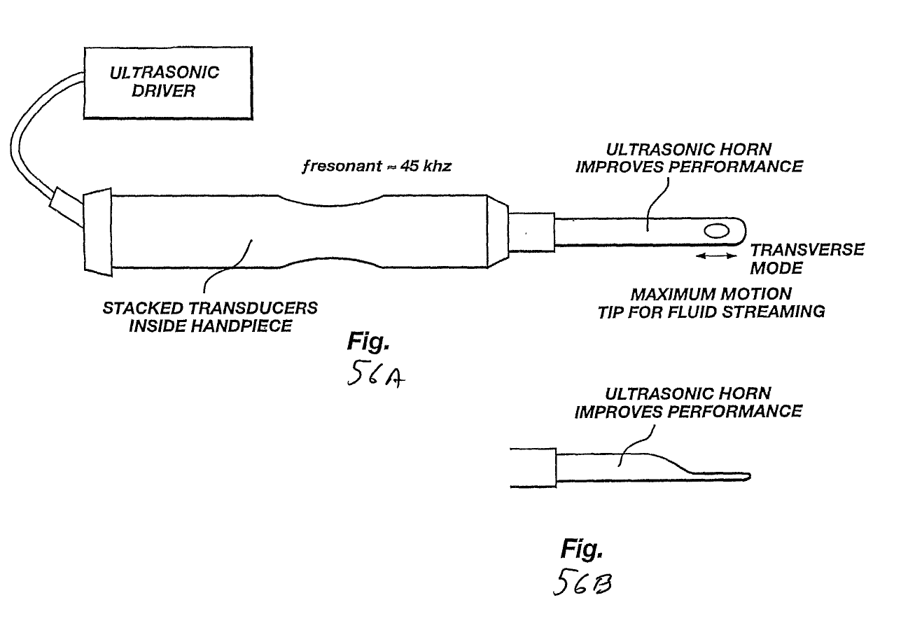

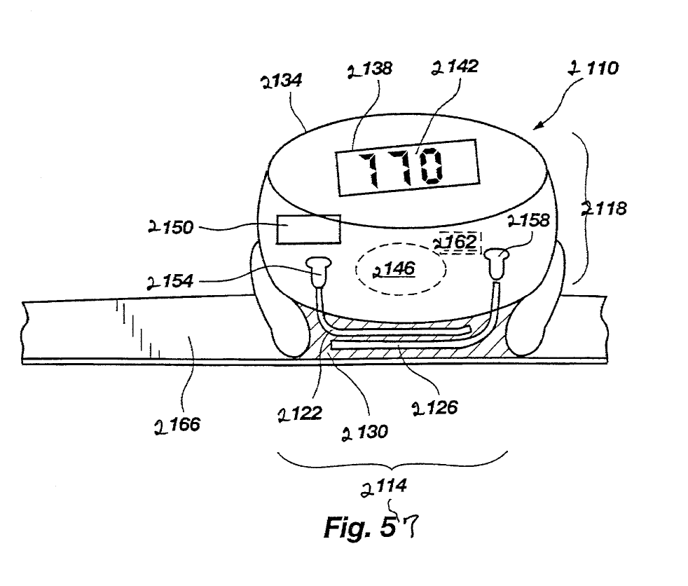

In one example from the '140 patent, FIGS. 57A and 57B illustrate a similar device 2206 with the added feature that the analysis is performed at the time of collection of the analyte. In this illustrative embodiment, glucose is the analyte to be collected and assayed by the glucose oxidase reaction described previously. A color reaction develops as the analyte is collected. The device 2206 includes a case 2210 attached to one or two straps 2214 so that the device can be worn on the wrist. Alternatively, the device 2206 may be attached by other conventional means to another part of the body. The case is composed of upper 2218 and lower 2222 sections connected by at least one hinge 2226. The sections 2218 and 2222 can pivot with respect to each other at the hinge 2226 and can be held together by a latch 2230. The lower section 2222 contains an opening through which glucose may pass from the skin to the interior of the device 2206, and enhancers and ultrasound energy may pass to the body. A collection/reaction pad 2234 on the interior of the device 2206 is positioned with respect to the opening in the lower section 2222 so that glucose entering the device 2206 from the skin is collected by the pad 2234. The pad 2234 also contains one or more enhancers, for increasing the permeability of the skin, and the reagents for performing the glucose oxidase assay of glucose concentration as described in Example 24, below. One or more ultrasonic transducers 2238 are positioned above the collection/reaction pad 2234. Ultrasound produced by the transducers 2238 helps the enhancer or enhancers to enter the skin and also helps draw glucose out of the skin toward the collection/reaction pad 2234. A battery 2242 is contained within the case 2210 as a power source for the transducers 2238. The case 2210 also contains a detector 2246 for detecting the results of the glucose oxidase assay, an LED 2250 for illuminating a liquid crystal display 2254, and electronic components 2258 for controlling the transducers 2238 and display of the results of the assay on the liquid crystal display 2254.

The detector 2246 for the glucose oxidase reaction is an optical device for reflectance reading. The electronic components 2258 are used to control the piezo transducer 2238 and calculate the results from the signal of the detector 2246. The transducer 2238 can be controlled to provide constant frequency and constant intensity, or can provide swept frequency, swept intensity, or both. The results of the glucose oxidase assay are displayed on a display 2254 such as an LCD display on the surface of the device 2206, the upper section 2218 of the case 2210 having an opening through which the display 2254 can be seen.

Also from the '140 patent, FIG. 58 shows an embodiment of a portable monitoring device for using this method of collecting and monitoring analytes with the aid of ultrasound and chemical enhancer. The monitoring device 2110 contains disposable 2114 and non-disposable 2118 units which are couplable together. The disposable unit 2114 contains a source fiber 2122, a detection fiber 2126, and a reservoir 2130 for holding a chemical enhancer. Both the source fiber 2122 and the detection fiber 2126 are optical fibers capable of transmitting light energy and have exposed on their exterior surfaces a binder capable of binding an analyte. The binder is selected according to the analyte that is to be monitored. The reservoir may be an absorbent paper or pad saturated with the enhancer formulation, or a liquid reservoir (e.g., a TheraDerm-LRS™ patch manufactured by TheraTech, Inc., Salt Lake City, Utah), or other suitable unit for containing the enhancer formulation. The non-disposable unit 2118 contains a cover 2134 for protecting the internal components of the non-disposable unit 2118. Built into the cover 2134 is an opening 2138 through which an LCD display 2142 is visible. In the interior of the non-disposable unit 2118 is an ultrasonic transducer 2146 which is connected to a power source, such as a battery 2150. The transducer 2146 is positioned to be over the reservoir 2130 so that, when the monitoring device 2110 is placed on the skin 2166 of an individual, the reservoir lies between the skin 2166 and the transducer 2146. Also in the interior of the non-disposable unit 2118 are a light source 2154, which may be an LED, laser diode, or other source of optical energy, and detector 2158 capable of converting the incoming optical energy into an electrical signal. The detector 2158 may be a photo diode, a photo multiplier tube, and the like. An integrated circuit 2162 is coupled to the transducer 2146, the light source 2154, the photosensitive diode 2158, the output device 2142, and the battery 2150. When the disposable unit 2114 and the non-disposable unit 2118 are coupled together, the source fiber 2122 is coupled to the light source 2154 so that light energy may be produced by the light source 2154 and transmitted through the source fiber 2122. Further, the detection fiber 2126 is coupled to the photosensitive diode 2158 so that light energy coupled into the detection fiber 2126 is conducted to the photosensitive diode 2158.

The monitoring device 2110 is operated by coupling the disposable unit 2114 and the non-disposable unit 2118 such that the source 2122 and detection 2126 fibers are, respectively, coupled to the light source 2154 and the photosensitive diode 2158. The binder attached to the outer surface on the source 2122 and detection 2126 fibers is selected for a desired analyte that is to be monitored. The chemical enhancer in the reservoir 2130 is also selectable. The monitoring device 2110 is then placed on the skin 2166 of the individual or animal to be monitored with the reservoir 2130 contacting the skin 2166. The transducer 2146 is activated to produce ultrasound, preferably in the frequency range of 0.1 to 100 MHz, more preferably 3-30 MHz, and most preferably 5-25 MHz. Optionally, frequency sweeping on other modulations from high to low frequencies can be employed to help drive the enhancers rapidly into the stratum corneum. The average intensity of the ultrasound is preferably in the range of 0.01-5 W/cm2, more preferably 0.05-3 W/cm2. However, higher instantaneous intensities can be employed if the average energy is kept low enough or cooling is applied to prevent damage to the transducer 2146 and/or the skin 2166. The ultrasound is applied for a time sufficient to drive chemical enhancer from the reservoir 2130 into the stratum corneum of the skin 2166. Five to twenty minutes or less is ordinarily sufficient to accomplish this. Once the enhancer has permeated the stratum corneum, an optional frequency modulation with the form of the frequency modulation designed to draw analytes from the body into the monitoring device 2110 may be applied. Analytes traverse the stratum corneum and are moved toward the transducer 2146. As the analytes pass through the disposable unit 2114 they come into close proximity to the source 2122 and detection 2126 fibers. Analytes of the specific type recognized by the binder are then bound on the surface of the fibers. At selected times, the integrated circuit 2162 transmits a signal to the light source 2154 to transmit light energy. This light energy is conducted through the source fiber 2122 and as it does so, evanescent wave energy passes through the wall of the source fiber 2122. A portion of this evanescent wave energy is coupled into the detection fiber; however the amount of cross coupling between the source and detection fibers is modulated by the presence of the specific analyte bound to the binder on the surface of both source 2122 and detection 2126 fibers. The evanescent wave energy captured by the detection fiber 2126 is then conducted to the photosensitive diode 2158. The photosensitive diode 2158 measures the intensity of the captured evanescent wave energy and transmits the intensity measurement to the integrate circuit 2162, where the information is stored. Once two or more intensity measurements made at distinct intervals are stored by the integrated circuit 2162, the integrated circuit 2162 determines the parameters describing the equilibrium curve and calculates the concentration of the specific analyte within the body of the individual. This calculated concentration is then transmitted by a signal to the output device 2142 which may comprise one or more of numerous embodiments. For example, the output device can be an LCD, LED, analog panel meter or an audio voice simulation to convey the data to the user. Other examples include an electronic data port, which may provide the output data in a format compatible with other electronic devices utilizing conducting cables to carry this information, a wireless radio frequency transmission, or a modulated light source, where the concentration is displayed or transmitted or otherwise made known thereon.

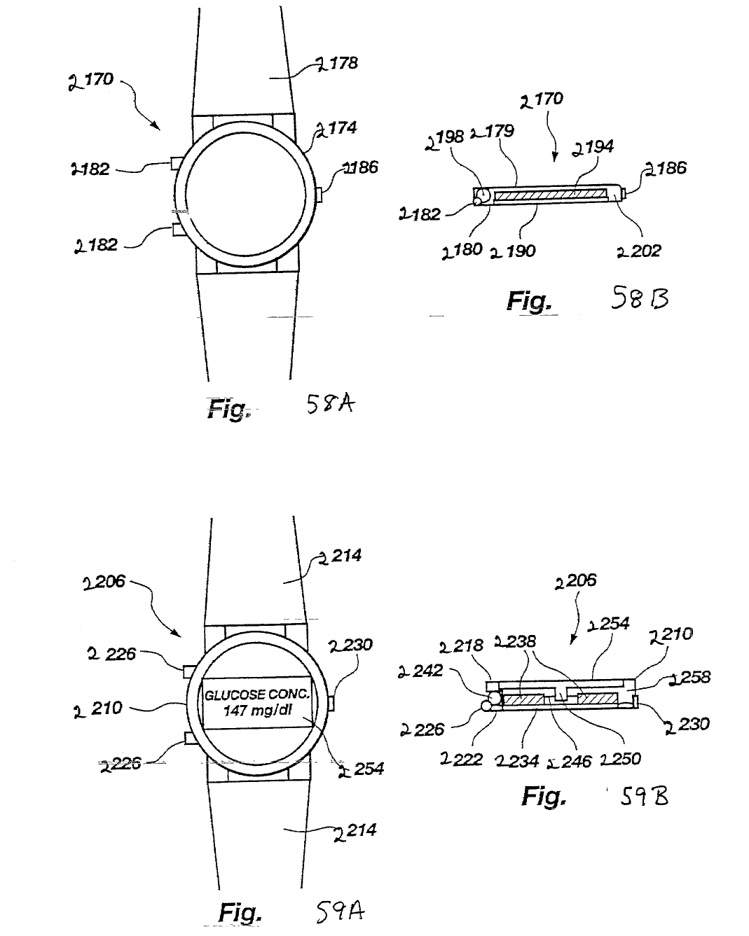

Also from the '140 patent, FIGS. 59A and 59B show an illustrative embodiment of a portable device 2170 for using this method for collection of the analyte with the aid of ultrasound and chemical enhancer. The device 2170 may be embodied in a form having the general appearance and size of a man's wrist watch. Accordingly, the device 2170 consists of a case 2174 attached to one or two wrist straps 2178. The case 2174 consists of upper 2179 and lower 2180 sections which are connected by at least one hinge 2182. The upper 2179 and lower 2180 sections are ordinarily held together by a latch 2186. Inside the case 2174 are a collecting pad 2190, an ultrasonic transducer 2194, a battery 2198, and assorted electronic components to drive the transducer, control the measurement cycles, process this data, and drive a display 2202. The lower section 2180 of the case 2174 includes an opening over which the collecting pad 2190 is positioned. Thus, when the device 2170 is strapped to a person's wrist, the collecting pad 2190 is in contact with the person's skin. The collecting pad 2190 collects the analyte that is drawn from the person's body and also may contain one or more enhancers for enhancing permeability of the skin. Alternatively, enhancers may be stored in a separate reservoir. The transducer 2194 is positioned within the case 2174 to be directly over the collecting pad 2190 so that when the device 2170 is strapped to a person's wrist, the collecting pad 2190 is between the wrist and the transducer 2194. A battery 2198 and electronic components 2202 are located within the case 2174 to provide, respectively, a power source and controls for operating the transducer 2194.

This device 2170 shown in FIGS. 59A and 59B is designed to be tightly strapped around the wrist so that the device 2170 is in intimate contact with the skin (or clamped to a fold of skin on the underside of the forearm). The collecting pad 2190 serves as a reservoir for collection of the analyte. In the embodiment illustrated in FIGS. 59A and 59B, the collecting pad 2190 also contains a chemical enhancer, one being selected that will not interfere with the subsequent analysis of the analyte of interest. The collecting pad 2190, which may be an absorbent pad such as a filter paper, is inserted into the device after the hinged upper 2179 and lower 2180 sections are opened. Closing the upper 2179 and lower 2180 sections positions the piezo transducer 2194 for producing an ultrasound signal next to the collecting pad 2190. In one embodiment, closing the upper 2179 and lower 2180 sections activates the device 2170 and starts a timer. After sufficient time has elapsed, the device 2170 shuts itself off and signals the user that the analyte has been collected. The collecting pad 2190 could then be removed for immediate or later analysis.

### EXAMPLE 24

An alternate method for detection of an analyte, such as glucose, following the sample collection through the porated skin surface as described above, can be achieved through the use of enzymatic means. Several enzymatic methods exist for the measurement of glucose in a biological sample. One method involves oxidizing glucose in the sample with glucose oxidase to generate gluconolactone and hydrogen peroxide. In the presence of a colorless chromogen, the hydrogen peroxide is then converted by peroxidase to water and a colored product.

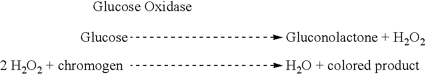

The intensity of the colored product will be proportional to the amount of glucose in the fluid. This color can be determined through the use of conventional absorbance or reflectance methods. By calibration with known concentrations of glucose, the amount of color can be used to determine the concentration of glucose in the collected analyte. By testing to deten-nine the relationship, one can calculate the concentration of glucose in the blood of the subject. This information can then be used in the same way that the information obtained from a blood glucose test from a finger puncture is used. Results can be available within five to ten minutes.

### EXAMPLE 25

Any system using a visual display or readout of glucose concentration will indicate to a diagnostician or patient the need for administration of insulin or other appropriate medication. In critical care or other situations where constant monitoring is desired and corrective action needs to be taken almost concurrently, the display may be connected with appropriate signal means which triggers the administration of insulin or other medication in an appropriate manner. For example, there are insulin pumps that are implanted into the peritoneum or other body cavity which can be activated in response to external or internal stimuli. Alternatively, utilizing the enhanced transdermal flux rates possible with micro-poration of the stratum corneum and other techniques described in this invention, an insulin delivery system could be inplemented transdermally, with control of the flux rates modulated by the signal from the glucose sensing system. In this manner a complete biomedical control system can be available which not only monitors and/or diagnoses a medical need but simultaneously provides corrective action.

Biomedical control systems of a similar nature could be provided in other situations such as maintaining correct electrolyte balances or administering analgesics in response to a measured analyte parameter such as prostaglandins.

### EXAMPLE 26

Similar to audible sound, sonic waves can undergo reflection, refraction, and absorption when they encounter another medium with dissimilar properties [D. Bommarman et al., 9 Pharm. Res. 559 (1992)]. Reflectors or lenses may be used to focus or otherwise control the distribution of sonic energy in a tissue of interest. For many locations on the human body, a fold of flesh can be found to support this system. For example, an earlobe is a convenient location that would allow use of a reflector or lens to assist in exerting directional control (e.g., “pushing” of analytes or permeants through the porated stratum corneum) similar to what is realized by changing sonic frequency and intensity.

### EXAMPLE 27

Multiple sonic energy transducers may be used to selectively direct the direction of transdermal flux through porated stratum corneum either into the body or from the body. A fold of skin such as an earlobe allow transducers to be located on either side of the fold. The transducers may be energized selectively or in a phased fashion to enhance transdermal flux in the desired direction. An array of transducers or an acoustic circuit may be constructed to use phased array concepts, similar to those developed for radar and microwave communications systems, to direct and focus the sonic energy into the area of interest.

### EXAMPLE 28

In this example, the procedure of Example 19 is followed with the exception that the heat-separated epidermis samples are first treated with an excimer laser (e.g. model EMG/200 of Lambda Physik; 193 nm wavelength, 14 ns pulse width) to ablate the stratum corneum according to the procedure described in U.S. Pat. No. 4,775,361, hereby incorporated by reference.

### EXAMPLE 29

In this example, the procedure of Example 19 is followed with the exception that the heat-separated epidermis samples are first treated with 1,1′-diethyl-4,4′-carbocyanine iodide (Aldrich, λₘₐₓ=703 nm) and then a total of 70 mJ/cm²/50 msec is delivered to the dye-treated sample with a model TOLD9150 diode laser (Toshiba America Electronic, 30 mW at 690 nm) to ablate the stratum corneum.

### EXAMPLE 30

In this example, the procedure of Example 29 is followed with the exception that the dye is indocyanine green (Sigma cat. no. 1-2633; λₘₐₓ=775 nm) and the laser is a model Diolite 800-50 (LiCONiX, 50 mW at 780 nm).

### EXAMPLE 31

In this example, the procedure of Example 29 is followed with the exception that is methylene blue and the laser is a model SDL-8630 (SDL Inc.; 500 mW at 670 nm).

### EXAMPLE 32

In this example, the procedure of Example 29 is followed with the exception that the dye is contained in a solution comprising a permeation enhancer, e.g. CE1.

### EXAMPLE 33

In this example, the procedure of Example 29 is followed with the exception that the dye and enhancer-containing solution are delivered to the stratum corneum with the aid of exposure to ultrasound.

### EXAMPLE 34

In this example, the procedure of Example 31 is followed with the exception that the pulsed light source is a short arc lamp emitting over the broad range of 400 to 1100 nm but having a bandpass filter placed in the system to limit the output to the wavelength region of about 650 to 700 nm.

### EXAMPLE 35

In this example, the procedure of Example 19 is followed with the exception that the heat-separated epidermis samples are first punctured with a microlancet (Becton Dickinson) calibrated to produce a micropore in the stratum corneum without reaching the underlying tissue.

### EXAMPLE 36

In this example, the procedure of Example 19 is followed with the exception that the heat-separated epidermis samples are first treated with focused sonic energy in the range of 70-480 mJ/cm²/50 ms to ablate the stratum corneum.

### EXAMPLE 37

In this example, the procedure of Example 19 is followed with the exception that the stratum corneum is first punctured hydraulically with a high pressure jet of fluid to form a micropore of up to about 100 μm diameter.

### EXAMPLE 38

In this example, the procedure of Example 19 is followed with the exception that the stratum corneum is first punctured with short pulses of electricity to form a micropore of up to about 100 μm diameter.

### EXAMPLE 39

Acoustic Streaming

A new mechanism and application of sonic energy in the delivering of therapeutic substances into the body and/or harvesting fluids from within the body into an external reservoir through micro-porations formed in the stratum corneum layer will now be described. An additional aspect of this invention is the utilization of sonic energy to create an acoustic streaming effect on the fluids flowing around and between the intact cells in the epidermis and dermis of the human skin. Acoustic streaming is a well documented mode by which sonic energy can interact with a fluid medium. Nyborg, Physical Acoustics Principles and Methods, p. 265-33 1, Vol 11-Part B, Academic Press, 1965, The first theoretical analysis of acoustic streaming phenomenon was given by Rayleigh (1884, 1945). In all extensive treatment of the subject, Longuet-Higgins (1953-1960) has given a result applicable to two dimensional flow that results in the near vicinity of any vibrating cylindrical surface. A three dimensional approximation for an arbitrary surface was developed by Nyborg (1958). As described by Fairbanks et al., 1975 Ultrasonics Symposium Proceedings, IEEE Cat. #75, CHO 9944SU, sonic energy, and the acoustic streaming phenomenon can be of great utility in accelerating the flux of a fluid through a porous medium, showing measurable increases in the flux rates by up to 50 times that possible passively or with only pressure gradients being applied.

All previous transdernal delivery or extraction efforts utilizing ultrasound have focused oil methods of interaction between the sonic energy and the skin tissues designed to permeabilize the stratum corneum (SC) layer. The exact mode of interaction involved has been hypothesized to be due exclusively to the local elevation of the temperature in the SC layer, and the resultant melting of the lipid domains in the intercellular spaces between the corneocytes. Srinivasan et al. Other researchers have suggested that micro-cavitations and or shearing of the structures in the stratum corneum opens up channels through which fluids may flow more readily. In general, the design of the sonic systems for the enhancement of transdermal flux rates has been based on the early realization that the application of an existing therapeutic ultrasound unit designed to produce a “deep-heating” effect on the subject, when used in conjunction with a topical application of a gelled or liquid preparation containing the drug to be delivered into the body, could produce a quantifiable increase in the flux rate of the drug into the body. In the context of the method taught herein to create micro-pores in this barrier layer, the use of sonic energy may now be thought of in a totally new and different sense than the classically defined concepts of sonophoresis.

Based on the experimental discovery mentioned in U.S. Pat. Nos. 5,458,140 and 5,445,611 that when a small hole existed or was created in the stratum corneum (SC) in the Franz cells used in the in vitro studies, that the application of an appropriately driven ultrasonic transducer to the fluid reservoir on either side of the porated SC sample, an “acoustic streaming” event could be generated wherein large flux rates of fluid where capable of being pumped through this porated membrane.

With the method taught herein to create the controlled micro-porations in the stratum corneum layer in the living subject's skin, the application of the fluid streaming mode of sonic/fluid interaction to the induction of fluid into or out of the body may now be practically explored. For example, clinical studies have shown that by making a series of four 80 μm diameter micro-pores in a 400 μm square, and then applying a mild (10 to 12 inches of Hg) suction to this area, an average of about 1 μl of interstitial fluid can be induced to leave the body for external collection in an external chamber. By adding a small, low power sonic transducer to this system, configured such that it actively generates inwardly converging concentric circular pressure waves in the 2 to 6 mm of tissue surrounding the poration site, it has been demonstrated that this ISF flux rate can be increased by 50%.

By relieving ourselves of the desire to create some form of direct absorption of sonic energy in the skin tissues (as required to generate heating), frequencies of sonic energy can be determined for which the skin tissues are virtually transparent, that is at the very low frequency region of 1 kHz to 500 kHz. Even at some of the lowest frequencies tested, significant acoustic streaming effects could be observed by using a microscope to watch an in vivo test wherein die subject's skin was micro-porated and ISF was induced to exit the body and pool on the surface of the skin. Energizing the sonic transducer showed dramatic visual indications of the amount of acoustic streaming as small pieces of particulate matter were carried along with the ISF as it swirled about. Typical magnitude of motion exhibited can be described as follows: for a 3 mm diameter circular pool of ISF on the surface of the skin, a single visual particle could be seen to be completing roughly 3 complete orbits per second. This equates to a linear fluid velocity of more than 2.5 mm/second. All of this action was demonstrated with sonic power levels into the tissues of less than 100 mW/cm².

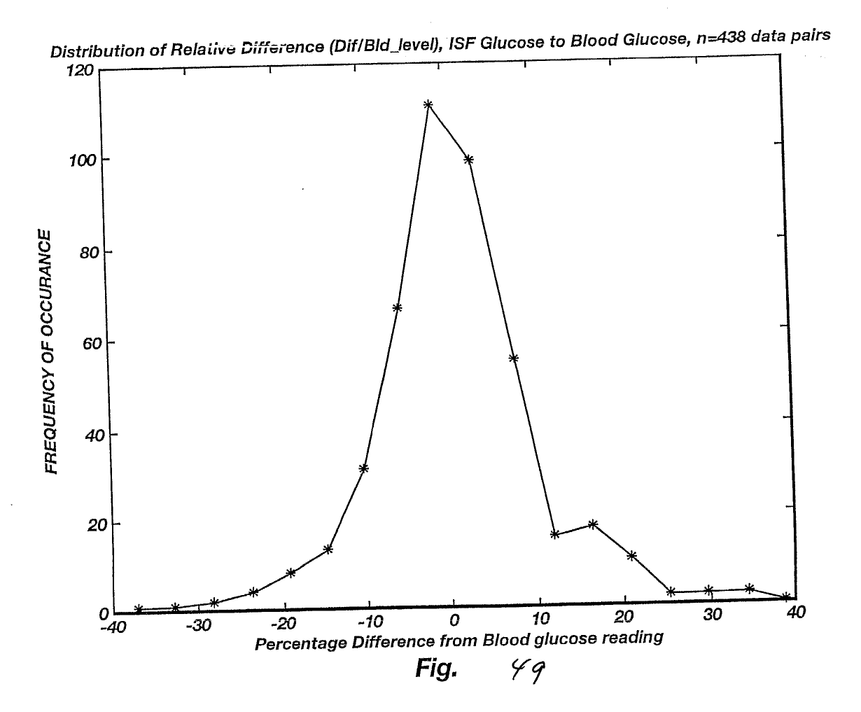

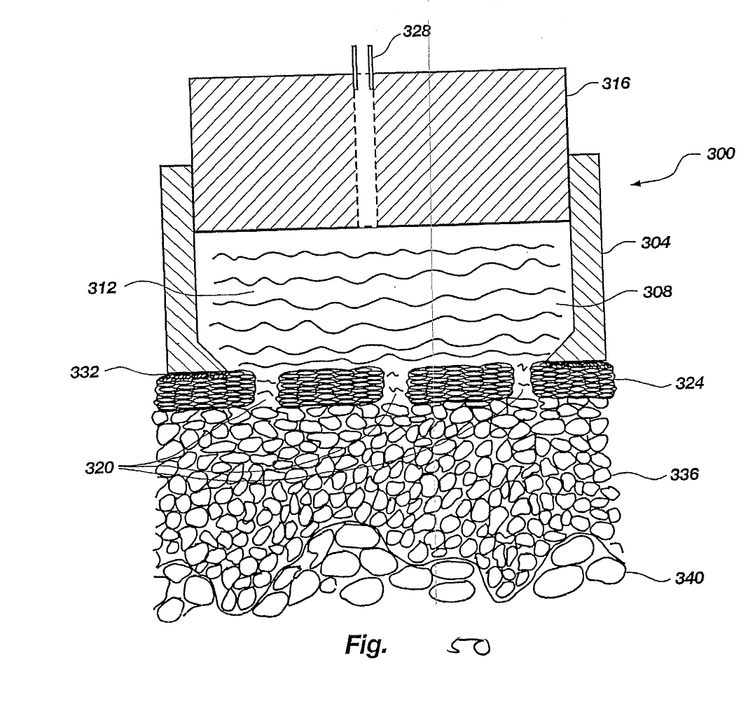

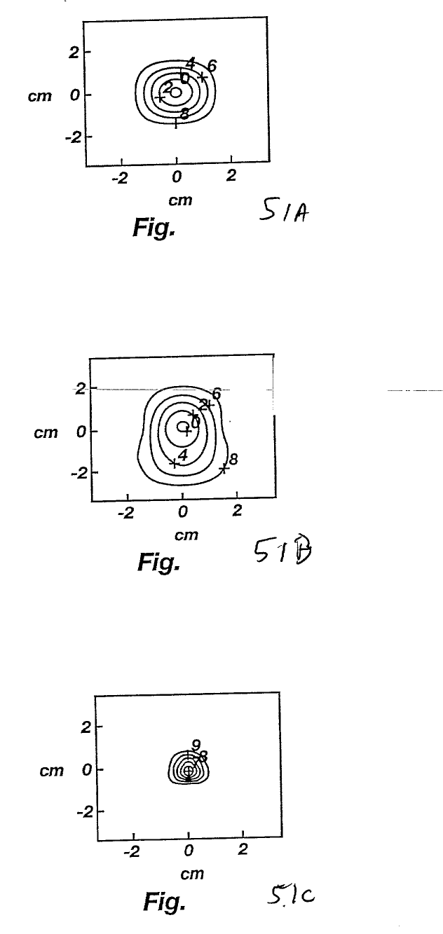

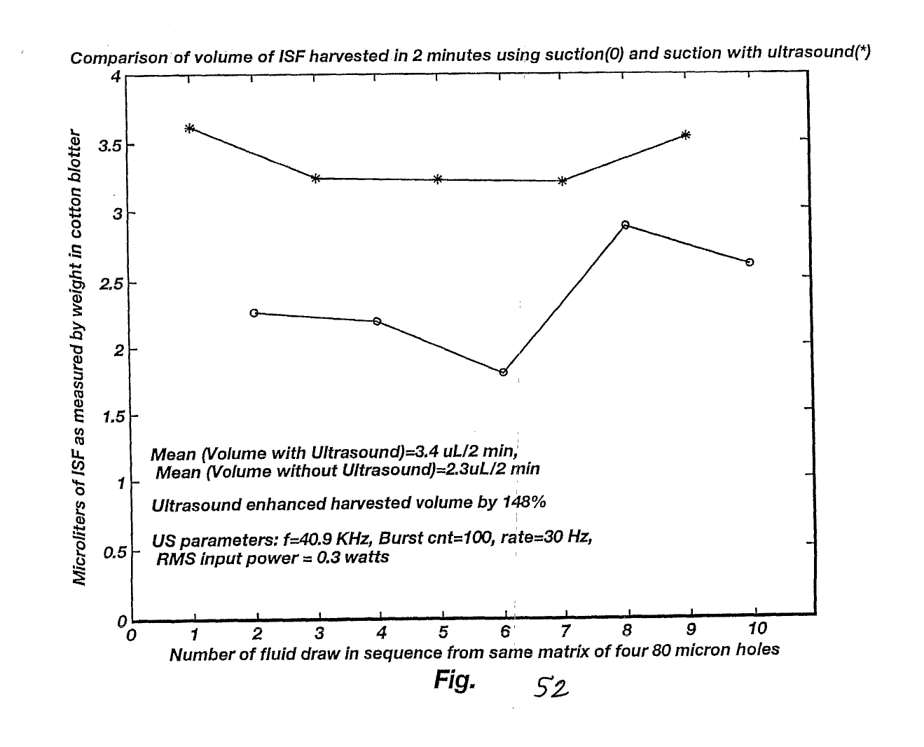

While one can easily view the top surface of the skin, and the fluidic activity thereon, assessing what is taking place dynamically within the skin tissue layers in response to the coupling into these tissues of sonic energy is much more difficult. One can assume, that if such large fluid velocities (e.g. >2.5 mm/S) may be so easily induced on the surface, then some noticeable increase in the fluid flow in the intercellular channels present in the viable dermal tissues could also be realized in response to this sonic energy input. Currently, an increase in harvested ISF through a given set of microporations when a low frequency sonic energy was applied to the area in a circle surrounding the poration sites has been quantified. In this experiment, an ISF harvesting technique based solely on a mild suction (10 to 12 inches of HG) was alternated with using the exact same apparatus, but with the sonic transducer engaged. Over a series of 10 two-minute harvesting periods, five with mere suction and five with both suction and sonic energy active, it was observed that by activating the sonic source roughly 50% more ISF was collectable in the same time period. These data are shown in FIG. 52. This increase in ISF flux rate was realized with no reported increase in sensation from the test subject due to the sonic energy. The apparatus used for this experiment is illustrated in FIGS. 53-55. The transducer assembly in FIGS. 53-55 is comprised of a thick walled cylinder of piezo-electric material, with an internal diameter of roughly 8 mm and a wall thickness of 4 mm. The cylinder has been polarized such that when an electrical field is applied across the metalized surfaces of the outer diameter and inner diameter, the thickness of the wall of the cylinder expands or contracts in response to the field polarity. In practice, this configuration results in a device which rapidly squeezes the tissue which has been suctioned into the central hole, causing an inward radial acoustic streaming effect on those fluids present in these tissues. This inward acoustic streaming is responsible for bringing more ISF to the location of the micro-porations in the center of the hole, where it can leave the body for external collection.

A similar device shown in FIG. 56A-B was built and tested and produced similar initial results. In the FIG. 56A-B version, an ultrasonic transducer built by Zevex, Inc. Salt Lake City, Utah, was modified by having a spatulate extension added to the sonic horn. A 4 mm. hole was placed in the 0.5 mm thick spatulate end of this extension. When activated, the principle motion is longitudinal along the length of the spatula, resulting in essentially a rapid back and forth motion. The physical perturbation of the metalic spatula casued by the placement of the 4 mm hole, results in a very active, but chaotic, large displacement behavior at this point. In use, the skin of the subject was suctioned up into this hole, and the sonic energy was then cunductined into the skin in a fashion similar to that illustrated in FIG. 33.

Novel aspects of this new application of ultrasound include the following basic areas:

1. The function of the ultrasound is no longer needed to be focused on permeabilizing the SC barrier membrane as taught by Langer, Kost, Bommannan and others.

2. A much lower frequency system can be utilized which has very little absorption in the skin tissues, yet can still create the fluidic streaming phenomenon desired within the intercellular passageways between the epidermal cells which contain the interstitial fluid.

3. The mode of interaction with the tissues and fluids therein, is the so-called “streaming” mode, recognized in the sonic literature as a unique and different mode than the classical vibrational interactions capable of shearing cell membranes and accelerating the passive diffusion process.

By optimizing the geometric configuration, frequency, power and modulations applied to the sonic transducer, it has been shown that significant increases in the fluid flux through the porated skin sites can be achieved. The optimization of these parameters is designed to exploit the non-linearities governing the fluid flow relationships in this microscopically scaled environment. Using frequencies under 200 KHz, large fluidic effects can be observed, without any detectable heating or other negative fissue interactions. The sonic power levels required to produce these measurable effects are very low, with average power levels typically under 100 milliwatts/cm².

Therefore, the above examples are but representative of systems which may be employed in certain embodiments relating to the utilization of ultrasound or ultrasound and chemical enhancers in the collection and quantification of analytes for diagnostic purposes and for the transdermal delivery of permeants. However, the invention is not limited only to the specific illustrations.

Various modifications and alterations of this invention will become apparent to those skilled in the art without departing from the scope and spirit of this invention, and it should be understood that this invention is not to be unduly limited to the illustrative embodiments set forth herein. For example, there are numerous poration techniques and enhancer systems, some of which may function better than another, for detection and withdrawn of certain analytes or delivery of permeants through the stratum corneum. Moreover, within the guidelines presented herein, a certain amount of experimentation can be readily carried out by those skilled in the art. Therefore, the invention is limited in scope only by the following claims and functional equivalents thereof.

What is claimed is:

1. A method for obtaining interstitial fluid for diagnostic testing comprising:
(a) porating a selected area of skin to form an opening for extracting a sample comprising interstitial fluid,which sample is suitable for quantitating an analyte;
(b) collecting the sample from the opening, wherein step (b) is enhanced by applying a vacuum to the selected area of the skin.

2. A method for obtaining biological fluid for diagnostic testing comprising:
(a) forming an opening in an area of skin suitable for extracting a sample of biological fluid suitable for measuring a characteristic of the fluid;
(b) extracting the sample from the opening, wherein at least one of positive and negative pressure is employed in order to enhance the extraction of the sample.

3. The method of claim 2 wherein the biological fluid comprises blood.

4. The method of claim 2 wherein the biological fluid comprises interstitial fluid.

5. A multi-layer integrated device comprising:
(a) a receiving layer capable of receiving a sample of biological fluid including an analyte and facilitating the movement of the fluid;
(b) an analyte sensor capable of detecting the presence of analyte or measuring the concentration of analyte in the fluid; and
(c) a substrate layer that is capable of being in contact with a processing circuit, wherein the receiving layer (a) is located underneath at least a portion of the substrate layer (c) and facilitates the movement of the biological fluid to the sensor (b); and further wherein said substrate layer (c) has at least one opening therein.

6. A multi-layer integrated device comprising:
(a) a receiving layer capable of receiving a sample of biological fluid including an analyte and facilitating the movement of the fluid;
(b) an analyte sensor capable of detecting the presence of analyte or measuring the concentration of analyte in the fluid;
(c) a substrate layer that is capable of being in contact with a processing circuit, and
(d) a bottom layer; wherein the receiving layer (a) is located underneath at least a portion of the substrate layer (c) and wherein said substrate layer (c) has at least one opening therein.

7. An integrated device comprising:
(a) a pad capable of receiving and transporting a biological sample containing an analyte;
(b) a detector for detecting the presence and/or quantitating the concentration of analyte in the sample, said mechanism capable of being in contact with a display for illustrating results of the detector; and
(c) a strap or adhesive tape for holding the pad to an area of skin surface, wherein the integrated device contains at least one opening suitable to allow the biological sample to contact the pad.

8. The integrated device of claim 7 wherein the pad contains a surfactant to faciliate tranport of the sample across the pad.

9. An integrated device for removing and testing a biological sample from the skin comprising;
(a) a lower section having at least one opening therein;
(b) a pad capable of collecting and transporting a biological sample containing an analyte; and
(c) a detector for determining the presense and/or quantity of the analyte, said detector capable of being in contact with a display for the results of the detector.

10. The integrated device of claim 9 wherein the pad contains a surfactant to facilitate transport of the sample across the pad.

11. An integrated fluid harvesting and analysis device, comprising:
(a) a first layer having a porating element disposed thereon, the porating element forming at least one opening in the tissue;
(b) a sensor positioned in fluid communication with the at least one opening in the tissue, the sensor being responsive to a biological fluid collected from the tissue to provide an indication of a characteristic of the biological fluid.

12. The device of claim 11, and further comprising a second layer overlying the first layer, the sensor being positioned between the first layer and the second layer.

13. The device of claim 11, wherein the porating element is a heat conducting element that is heatable such that the temperature of tissue-bound water and other vaporizable substances in a selected area of the surface of the tissue proximate the heat conducting element is elevated above the vaporization point of water and other vaporizable substances thereby removing the surface of the tissue in said selected area.

14. The device of claim 13, wherein the porating element comprises a quantity of photothermal material that is responsive to application of optical energy to heat up and conduct heat to the surface of the tissue for forming at least one opening therein.

15. The device of claim 14, wherein said second layer comprises a portion which is transparent to optical energy.

16. The device of claim 11, wherein the porating element comprises at least one mechanical puncturing member protruding from a bottom surface of the first layer.

17. The device of claim 11, wherein the sensor comprises an electrochemical biosensor which is responsive to a level of glucose in interstitial fluid.

18. The device of claim 11, wherein the sensor comprises a calorimetric sensor that provides an indication of glucose level in interstitial fluid.

19. The device of claim 11, wherein the sensor comprises an assay pad having a reactive area that is positioned with respect to the at least one opening in the first layer tissue to absorb biological fluid collected therethrough.

20. The device of claim 19, and further comprising a mesh layer overlying the assay pad to transport biological fluid onto and across the reactive area of the assay pad.

21. The device of claim 20, wherein the mesh layer is treated with a surfactant substance to enhance the transport of biological fluid onto and across the reactive area of the assay pad.

22. The device of claim 19, wherein the sensor comprises an anode, a cathode, a reference electrode and a sense electrode, each of which is electrically coupled to the reactive area of the assay pad.

23. The device of claim 19, wherein the assay pad of the sensor is disposed along at least portion of an inner wall that extends perpendicular to a surface of the first layer.

24. The device of claim 11, and further comprising an sonic transducers formed of a compliant sonic transmissive material positioned above the layer to deliver sonic energy to the tissue surrounding a location of the tissue where the at least one opening is to be formed.

25. The device of claim 24, wherein the sonic transducer is formed of a compliant silicone material.

26. The device of claim 24, and comprising at least two sonic transducers positioned on opposite sides of the sensor.

27. The device of claim 11, and further comprising a sonic transducer formed of a compliant sonic transmissive material positioned above the layer to deliver sonic energy to the tissue surrounding a location of the tissue where the at least one opening is to be formed.

28. The device of claim 11, wherein the porating element comprises at least one electrically energized heat conducting element.

29. The device of claim 28, and further comprising at least two conductors embedded in the tissue-contacting layer and the at least one electrically energized heat conducting element being connected to the conductors for supplying electric current to the at least one electrically heatable element.

30. In combination, the integrated device of claim 11, and a mechanical element having a small opening therein and capable or receiving the integrated device such that the probe is aligned with the small opening, the mechanical element responsive to downward force thereon to cause the surface of the tissue to bulge into the small opening.

31. In combination, the integrated device of claim 11, and sealing means for pneumatically sealing the integrated device to the surface of the tissue and forming a sealed chamber, and means coupled to the sealing means for supplying negative pressure to the sealed chamber.

32. The combination of claim 31, and further comprising a sealed electrical connection to the sensor and/or probe via the sealing means.

33. The device of claim 12, and further comprising a fluid management chamber in a region of the integrated device between the first layer and the second layer, wherein surfaces in the fluid management chamber are treated with a chemical substance so as to facilitate the flow of biological fluid to the sensor.

34. The device of claim 33, wherein surface portions of the layer are coated with hydrophobic substances.

35. The device of claim 11, and further comprising a sense electrode coupled to the sensor to facilitate determination that the sensor is sufficiently wetted with biological fluid.

36. The device of claim 11, and further comprising means for coupling sonic energy through the device to the tissue.

37. The device of claim 36, and further comprising control means for controlling parameters of the sonic energy so that the sonic energy is adjusted to optimize each stage of a harvesting and analysis process.

38. An integrated fluid harvesting and analysis device, comprising:
(a) a first layer for positioning in contact with tissue and through which poration of tissue is achieved such that at least one opening is formed in the first layer and at least one opening is formed in the tissue;
(b) a sensor positioned in fluid communication with the at least one opening of the first layer, the sensor being responsive to a biological fluid collected from the tissue to provide an indication of a characteristic of the biological fluid.

39. The device of claim 38, and further comprising a second layer overlying the first layer, the sensor being positioned between the first layer and the second layer.

40. A method for harvesting biological fluid from tissue and analyzing the biological fluid, comprising steps of:
(a) placing a layer in contact with a surface of tissue;
(b) forming at least one hole in the tissue;
(c) collecting biological fluid from the tissue through at least one opening in the layer; and
(d) wetting a sensor that is positioned in fluid communication with the at least one opening in the layer with biological fluid to measure a characteristic of the biological fluid.

41. The method of claim 40, wherein the step of forming the at least one opening in the layer and the at least opening in the tissue comprises applying optical energy to a photothermal material on the layer that is contact with the tissue to thermally ablate the tissue and form the at least one opening therein.

42. The method of claim 40, wherein the step of forming the at least one opening in the layer and the at least one opening in the tissue comprises applying electrical energy to a heat conducting element on the layer that is in contact with the tissue to thermally ablate the tissue and form the at least one opening therein.

43. The method of claim 40, wherein the step of forming a hole in the tissue comprises forming at least one hole through the layer and into the tissue, wherein biological fluid from the tissue is collected through the hole in the layer.

44. The method of claim 40, wherein the step of forming a hole in the tissue comprises forming at least one hole in the tissue adjacent to the layer.

45. The method of claim 40, wherein the step of measuring comprises measuring an electrical characteristic of the sensor.

46. The method of claim 40, wherein the step of measuring comprises measuring a characteristic of light reflected from the sensor.

47. The method of claim 40, and further comprising the step of applying positive pressure to the layer so as to induce flow of biological fluid through an inlet port.

48. The method of claim 40, and further comprising the step of creating a negative pressure above the layer so as to draw biological fluid into the inlet port.

49. The method of claim 48, and further comprising the step of forming a sealed chamber over the layer and the sensor.
